(19) United States
(12) Patent Application Publication
(10) Pub. No.: US 2022/0273678 A1
(43) Pub. Date: Sep. 1, 2022

(54) Compositions and Methods for Inhibiting and Treating Coronavirus Infections

(71) Applicant: The Regents of the University of California, Oakland, CA (US)

(72) Inventors: Theodoros Kelesidis, Encino, CA (US); Vaithilingaraja Arumugaswami, Los Angeles, CA (US); Gustavo Garcia, JR., Gardena, CA (US)

(21) Appl. No.: 17/570,737

(22) Filed: Jan. 7, 2022

Related U.S. Application Data
(63) Continuation-in-part of application No. PCT/US2021/040869, filed on Jul. 8, 2021.
(60) Provisional application No. 63/050,786, filed on Jul. 11, 2020.
(60) Provisional application No. 63/124,841, filed on Dec. 13, 2020.
(60) Provisional application No. 63/166,202, filed on Mar. 25, 2021.

(51) Int. Cl. A61K 31/66; A61P 31/14; A61K 38/10

(52) U.S. Cl. CPC A61K 31/66; A61P 31/14; A61K 38/10

(57) ABSTRACT

Disclosed herein are methods of using one or more triphenylphosphonium compounds, to prevent, inhibit, and/or treat infections and symptoms caused by infection by a coronavirus, such as SARS-CoV-2.

### CROSS-REFERENCE TO RELATED APPLICATIONS

This application is a continuation-in-part of PCT/US2021/040869 filed Jul. 8, 2021, which claims the benefit of U.S. Patent Application No. 63/050,786 filed Jul. 11, 2020, U.S. Patent Application No. 63/124,841 filed Dec. 13, 2020, and U.S. Patent Application No. 63/166,202 filed Mar. 25, 2021, all of which are herein incorporated by reference in their entirety.

### BACKGROUND OF THE INVENTION

### 1. Field of the Invention

The field of the invention generally relates to compositions and methods for inhibiting and/or treating infections by coronaviruses, e.g., SARS-CoV-2.

### 2. Description of the Related Art

SARS-CoV-2 has rapidly spread throughout the world becoming a pandemic. Vaccines may have suboptimal efficacy, especially in immunocompromised patients, unclear long-term safety (like mRNA vaccines) and may not be accepted uniformly by all people. Current antivirals against SARS-CoV-2 have major limitations and cannot be used long term in high-risk groups such as immunocompromised patients. Thus, there is an urgent need to develop an efficacious, oral, safe, novel therapeutic strategy to protect from development of COVID-19 while having favorable impact on comorbidities.

### SUMMARY OF THE INVENTION

In some embodiments, the present invention is directed to methods of preventing, inhibiting, or reducing infection by a coronavirus in a cell or a subject, which comprises administering one or more mitochondrial targeted antioxidants and/or one or more ApoA-I mimetic peptides to the cell or the subject. In some embodiments, the present invention is directed to methods of preventing, inhibiting, or reducing infection by a coronavirus in a subject, which comprises providing a plasma concentration of about 2 ng/ml or higher of one or more mitochondrial targeted antioxidants in the subject before, during, and/or after the subject is exposed to the coronavirus by administering a therapeutically effective amount one or more mitochondrial targeted antioxidants to the subject. In some embodiments, the present invention is directed to methods of preventing, inhibiting, or reducing an inflammatory response caused by infection by a coronavirus in a cell or a subject, which comprises administering one or more mitochondrial targeted antioxidants and/or one or more ApoA-I mimetic peptides to the cell or the subject. In some embodiments, the present invention is directed to methods preventing, inhibiting, or reducing apoptosis caused by infection by a coronavirus in a cell or a subject, which comprises administering one or more mitochondrial targeted antioxidants and/or one or more ApoA-I mimetic peptides to the cell or the subject. In some embodiments, the present invention is directed to methods treating a subject for a coronavirus disease caused by infection by a coronavirus, which comprises administering one or more mitochondrial targeted antioxidants and/or one or more ApoA-I mimetic peptides to the subject. The one or more mitochondrial targeted antioxidants may be Mito-MES. The one or more ApoA-I mimetic peptides may be 4F, D-4F, reverse D-4F, or 6F. In some embodiments, the amount of the one or more mitochondrial targeted antioxidants administered to the subject is about 0.05 mg/kg to about 15 mg/kg, preferably about 0.2 mg/kg to about 1.5 mg/kg, or more preferably about 0.3 mg/kg to about 0.7 mg/kg weight of the subject. In some embodiments, the amount of the one or more mitochondrial targeted antioxidants administered to the subject is about 1-1000 mg, 5-100 mg, 10-80 mg, or 20-40 mg, and preferably about 20 mg. In some embodiments, the amount of the one or more mitochondrial targeted antioxidants administered to the subject is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some embodiments, the amount of the one or more ApoA-I mimetic peptides administered to the subject is about 0.02-15 mg/kg, about 0.15-10 mg/kg, about 1.5-10 mg/kg, about 1.0-2.0 mg/kg, about 0.01-7.5 mg/kg, about 0.05-5.0 mg/kg, about 0.75-5.0 mg/kg, or about 0.5-1.0 mg/kg weight of the subject. In some embodiments, the amount of the one or more ApoA-I mimetic peptides administered to the subject is about 1-500 mg, about 10-500 mg, about 100-500 mg, about 1-250 mg, about 10-250 mg, or about 100-250 mg. In some embodiments, the amount of the one or more mitochondrial targeted antioxidants and/or the amount of the one or more ApoA-I mimetic peptides is administered daily. In some embodiments, the one or more mitochondrial targeted antioxidants and/or one or more ApoA-I mimetic peptides is administered orally, subcutaneously, or intravenously, preferably orally. In some embodiments, the administration of the one or more mitochondrial targeted antioxidants and/or one or more ApoA-I mimetic peptides is before, during, and/or after the subject was exposed or likely exposed to the coronavirus. In some embodiments, the administration of the one or more mitochondrial targeted antioxidants and/or one or more ApoA-I mimetic peptides occurs for at least 1, 2, 3, 4, 5, 6, 7, 8, 9, 10, 11, 12, 13, or 14 days, after the likely or confirmed exposure to the coronavirus. In some embodiments, the administration of the one or more mitochondrial targeted antioxidants and/or one or more ApoA-I mimetic peptides occurs for at least 1-10 days after the exposure or likely exposure to the coronavirus. In some embodiments, the administration of the one or more mitochondrial targeted antioxidants and/or one or more ApoA-I mimetic peptides occurs for at least 1-10 days before the exposure or likely exposure to the coronavirus. In some embodiments, the coronavirus is SARS-CoV-2. In some embodiments, one or more Nrf2 agonists, e.g., dimethyl fumarate (DMF), may be administered to the cell or the subject. In some embodiments, the amount of the one or more Nrf2 agonists that is administered to the subject is about 0.02-8.0 mg/kg, about 0.15-8.0 mg/kg, about 4.0-8.0 mg/kg, about 0.01-4.0 mg/kg, about 0.1-4.0 mg/kg, or about 2.0-4.0 mg/kg weight of the subject. In some embodiments, the amount of the one or more Nrf2 agonists that is administered to the subject is about 1-480 mg, about 10-480 mg, about 240-480 mg, about 0.5-240 mg, about 5-240 mg, or about 120-240 mg. In some embodiments, administration of the one or more ApoA-I mimetic peptides and/or the one or more Nrf2 agonists is concurrent with the one or more mitochondrial targeted antioxidants. In some embodiments where both one or more mitochondrial targeted antioxidants and one or more ApoA-I mimetic peptides are administered (without the administration of an Nrf2 agonist), the ratio of the amount of the one or more mitochondrial targeted antioxidants to the amount of the one or more ApoA-I mimetic peptides that is administered ranges from about 0.0001:1 to about 100:1, preferably about 0.1:1 by molecular amount (moles). In some embodiments where one or more mitochondrial targeted antioxidants, one or more ApoA-I mimetic peptides, and one or more Nrf2 agonists are administered, the ratio of the amount of the one or more mitochondrial targeted antioxidants to the amount of the one or more ApoA-I mimetic peptides that is administered ranges from about 0.001:1 to about 10:1, preferably about 0.01:1, by molecular amount (moles). In some embodiments where one or more mitochondrial targeted antioxidants+one or more ApoA-I mimetic peptides+one or more Nrf2 agonists are administered, the relative amounts of the one or more mitochondrial targeted antioxidants:the one or more ApoA-I mimetic peptides:the one or more Nrf2 agonists that are administered is about 0.1:1:10 by molecular amount (moles).

In some embodiments, the invention is directed to the use of one or more mitochondrial targeted antioxidants and/or one or more ApoA-I mimetic peptides or a composition thereof (1) as an antiviral against a coronavirus; (2) to prevent, inhibit, and/or reduce apoptosis caused by infection by a coronavirus; (3) in the treatment of an infection by a coronavirus or in the manufacture of a medicament for treating the infection; (4) in the treatment of apoptosis and/or inflammation caused by a coronavirus or in the manufacture of a medicament for treating and/or inflammation caused by the coronavirus. In some embodiments, the use of one or more mitochondrial targeted antioxidants and/or one or more ApoA-I mimetic peptides is to prevent, inhibit, reduce, and/or treat (a) an infection by, (b) an inflammatory response caused by, (c) apoptosis caused by, (d) injury to lung tissue caused by, or (e) a disease caused by a coronavirus, such as SARS-CoV-2. In some embodiments, the use of one or more mitochondrial targeted antioxidants and/or one or more ApoA-I mimetic peptides is for the manufacture of a medicament for preventing, inhibiting, reducing, and/or treating (a) an infection by, (b) an inflammatory response caused by, (c) apoptosis caused by, (d) injury to lung tissue caused by, or (e) a disease caused by a coronavirus, such as SARS-CoV-2. In some embodiments, the one or more mitochondrial targeted antioxidants is used concurrently with one or more ApoA-I mimetic peptides and/or one or more Nrf2 agonists. The one or more mitochondrial targeted antioxidants may be Mito-MES. The one or more ApoA-I mimetic peptides may be 4F, D-4F, reverse D-4F, or 6F. In some embodiments, the amount of the one or more mitochondrial targeted antioxidants is about 1-1000 mg, 5-100 mg, 10-80 mg, or 20-40 mg, preferably about 20 mg. In some embodiments, the amount of the one or more mitochondrial targeted antioxidants is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some embodiments, the amount of the one or more ApoA-I mimetic peptides is about 1-500 mg, about 10-500 mg, about 100-500 mg, about 1-250 mg, about 10-250 mg, or about 100-250 mg. In some embodiments, up to 1000 mg of the one or more mitochondrial targeted antioxidants is provided as several divided doses. In some embodiments, the one or more mitochondrial targeted antioxidants, the one or more ApoA-I mimetic peptides, or a medicament comprising one or both is formulated for oral administration. In some embodiments, the coronavirus is SARS-CoV-2. In some embodiments, one or more Nrf2 agonists, e.g., DMF, is provided in combination with the one or more mitochondrial targeted antioxidants and/or the one or more ApoA-I mimetic peptides. In some embodiments, the amount of the one or more Nrf2 agonists is about 1-480 mg, about 10-480 mg, about 240-480 mg, about 0.5-240 mg, about 5-240 mg, or about 120-240 mg. In some embodiments, the amount of the one or more mitochondrial targeted antioxidants, the amount of the one or more ApoA-I mimetic peptides, and/or the one or more Nrf2 agonists is provided as a daily dose. In some embodiments where both one or more mitochondrial targeted antioxidants and one or more ApoA-I mimetic peptides are provided (without an Nrf2 agonist), the ratio of the amount of the one or more mitochondrial targeted antioxidants to the amount of the one or more ApoA-I mimetic peptides ranges from about 0.0001:1 to about 100:1, preferably about 0.1:1 by molecular amount (moles). In some embodiments where one or more mitochondrial targeted antioxidants, one or more ApoA-I mimetic peptides, and one or more Nrf2 agonists are provided, the ratio of the amount of the one or more mitochondrial targeted antioxidants to the amount of the one or more ApoA-I mimetic peptides ranges from about 0.001:1 to about 10:1, preferably about 0.01:1, by molecular amount (moles). In some embodiments where one or more mitochondrial targeted antioxidants+one or more ApoA-I mimetic peptides+one or more Nrf2 agonists are provided, the relative amounts of the one or more mitochondrial targeted antioxidants:the one or more ApoA-I mimetic peptides:the one or more Nrf2 agonists is about 0.1:1:10 by molecular amount (moles).

In some embodiments, the invention is directed to a composition comprising, consisting essentially of, or consisting of one or more mitochondrial targeted antioxidants in combination with: (a) one or more ApoA-I mimetic peptides, (b) one or more Nrf2 agonists, or (c) one or more ApoA-I mimetic peptides and one or more Nrf2 agonists. In some embodiments, the one or more mitochondrial targeted antioxidants is selected from mitoquinone and salts thereof, mitoquinol and salts thereof, SkQ1, Elamipretide, and Mito-TEMPO. In some embodiments, the one or more mitochondrial targeted antioxidants is Mito-MES. In some embodiments, the one or more ApoA-I mimetic peptides is selected from 4F, D-4F, reverse D-4F, and 6F. In some embodiments, the one or more Nrf2 agonists is selected from Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF). In some embodiments, the one or more Nrf2 agonists is DMF. In some embodiments, the composition comprises Mito-MES+4F, D-4F, reverse D-4F, or 6F. In some embodiments, the composition comprises Mito-MES+DMF. In some embodiments, the composition comprises (1) Mito-MES, (2) 4F, D-4F, reverse D-4F, or 6F, and (3) DMF. In some embodiments, the composition comprises about 1-1000 mg, 5-100 mg, 10-80 mg, or 20-40 mg, and preferably about 20 mg, of the one or more mitochondrial targeted antioxidants. In some embodiments, the composition comprises about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about or 200 mg of the one or more mitochondrial targeted antioxidants. In some embodiments, the composition comprises about 1-500 mg, about 10-500 mg, about 100-500 mg, about 1-250 mg, about 10-250 mg, or about 100-250 mg of the one or more ApoA-I mimetic peptides. In some embodiments, the composition comprises about 1-480 mg, about 10-480 mg, about 240-480 mg, about 0.5-240 mg, about 5-240 mg, or about 120-240 mg of the one or more Nrf2 agonists. In some embodiments, the composition comprises (a) about 1-1000 mg, 5-100 mg, 10-80 mg, or 20-40 mg, and preferably about 20 mg, of the one or more mitochondrial targeted antioxidants and (b) about 1-500 mg, about 10-500 mg, about 100-500 mg, about 1-250 mg, about 10-250 mg, or about 100-250 mg of the one or more ApoA-I mimetic peptides. In some embodiments, the composition comprises (a) about 1-1000 mg, 5-100 mg, 10-80 mg, or 20-40 mg, and preferably about 20 mg, of the one or more mitochondrial targeted antioxidants, (b) about 1-500 mg, about 10-500 mg, about 100-500 mg, about 1-250 mg, about 10-250 mg, or about 100-250 mg of the one or more ApoA-I mimetic peptides, and (c) about 1-480 mg, about 10-480 mg, about 240-480 mg, about 0.5-240 mg, about 5-240 mg, or about 120-240 mg of the one or more Nrf2 agonists. In some embodiments of compositions comprising one or more mitochondrial targeted antioxidants+one or more ApoA-I mimetic peptides (without an Nrf2 agonist), the ratio of the amount of the one or more mitochondrial targeted antioxidants to the amount of the one or more ApoA-I mimetic peptides ranges from about 0.0001:1 to about 100:1, preferably about 0.1:1 by molecular amount (moles). In some embodiments of compositions comprising one or more mitochondrial targeted antioxidants+one or more ApoA-I mimetic peptides+one or more Nrf2 agonists, the ratio of the amount of the one or more mitochondrial targeted antioxidants to the amount of the one or more ApoA-I mimetic peptides ranges from about 0.001:1 to about 10:1, preferably about 0.01:1, by molecular amount (moles). In some embodiments of compositions comprising one or more mitochondrial targeted antioxidants+one or more ApoA-I mimetic peptides+one or more Nrf2 agonists, the relative amounts of the one or more mitochondrial targeted antioxidants:the one or more ApoA-I mimetic peptides:the one or more Nrf2 agonists is about 0.1:1:10 by molecular amount (moles).

In some embodiments, the present invention is directed to methods of preventing, inhibiting, or reducing infection by a coronavirus in a cell or a subject, which comprises administering one or more TPP Compounds alone or in combination with one or more ApoA-I mimetic peptides and/or one or more Nrf2 agonists to the cell or the subject. In some embodiments, the present invention is directed to methods of preventing, inhibiting, or reducing infection by a coronavirus in a subject, which comprises providing a plasma concentration of about 2 ng/ml or higher of one or more TPP Compounds in the subject before, during, and/or after the subject is exposed to the coronavirus by administering a therapeutically effective amount one or more TPP Compounds to the subject. In some embodiments, the present invention is directed to methods of preventing, inhibiting, or reducing an inflammatory response caused by infection by a coronavirus in a cell or a subject, which comprises administering one or more TPP Compounds alone or in combination with one or more ApoA-I mimetic peptides and/or one or more Nrf2 agonists to the cell or the subject. In some embodiments, the present invention is directed to methods preventing, inhibiting, or reducing apoptosis caused by infection by a coronavirus in a cell or a subject, which comprises administering one or more TPP Compounds alone or in combination with one or more ApoA-I mimetic peptides and/or one or more Nrf2 agonists to the cell or the subject. In some embodiments, the present invention is directed to methods treating a subject for a coronavirus disease caused by infection by a coronavirus, which comprises administering one or more TPP Compounds alone or in combination with one or more ApoA-I mimetic peptides and/or one or more Nrf2 agonists to the subject. In some embodiments, the one or more TPP Compounds is a TPP Hydrocarbon or a TPP Conjugate. In some embodiments, the one or more TPP Compounds is Mito-MES. In some embodiments, the one or more TPP Compounds is dTPP. In some embodiments, dTPP and coenzyme Q10 are administered in the form of a mixture (i.e., instead of a TPP Conjugate, wherein the bioactive moiety is coenzyme Q10). In some embodiments the one or more TPP Compounds and the one or more Nrf2 agonists are provided in the form of a conjugate, i.e., a TPP Conjugate, wherein the bioactive moiety is an Nrf2 agonist. In some embodiments, the one or more TPP Compounds is not Mito-MES, MitoQ™, SkQ1 (Mitotech, S.A.), Elamipretide (Stealth BioTherapeutics), Mito-TEMPO (CAS 1569257-94-8) or a compound disclosed in U.S. Pat. Nos. 8,518,915; 9,192,676; 9,328,130; 9,388,156; US20070161609; US20070225255; US20080161267; US20100168198; US20160200749; US20180305328; US20190248816; US20190330249; US20190374558; WO2005019232; WO2006005759; WO2007046729; WO2008145116; WO2015063553; WO2017106803; and WO2018162581. The one or more ApoA-I mimetic peptides may be 4F, D-4F, reverse D-4F, or 6F. In some embodiments, the amount of the one or more TPP Compounds administered to the subject is about 0.05 mg/kg to about 15 mg/kg, preferably about 0.2 mg/kg to about 1.5 mg/kg, or more preferably about 0.3 mg/kg to about 0.7 mg/kg weight of the subject. In some embodiments, the amount of the one or more TPP Compounds administered to the subject is about 1-1000 mg, 5-100 mg, 10-80 mg, or 20-40 mg, and preferably about 20 mg. In some embodiments, the amount of the one or more TPP Compounds administered to the subject is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some embodiments, the amount of the one or more ApoA-I mimetic peptides administered to the subject is about 0.02-15 mg/kg, about 0.15-10 mg/kg, about 1.5-10 mg/kg, about 1.0-2.0 mg/kg, about 0.01-7.5 mg/kg, about 0.05-5.0 mg/kg, about 0.75-5.0 mg/kg, or about 0.5-1.0 mg/kg weight of the subject. In some embodiments, the amount of the one or more ApoA-I mimetic peptides administered to the subject is about 1-500 mg, about 10-500 mg, about 100-500 mg, about 1-250 mg, about 10-250 mg, or about 100-250 mg. In some embodiments, the amount of one or more TPP Compounds, the one or more ApoA-I mimetic peptides, and/or the one or more Nrf2 agonists is administered daily. In some embodiments, the one or more TPP Compounds, the one or more ApoA-I mimetic peptides, and/or the one or more Nrf2 agonists is administered orally, subcutaneously, or intravenously, preferably orally. In some embodiments, the administration of the one or more TPP Compounds, the one or more ApoA-I mimetic peptides, and/or the one or more Nrf2 agonists is before, during, and/or after the subject was exposed or likely exposed to the coronavirus. In some embodiments, the administration of the one or more TPP Compounds, the one or more ApoA-I mimetic peptides, and/or the one or more Nrf2 agonists occurs for at least 1, 2, 3, 4, 5, 6, 7, 8, 9, 10, 11, 12, 13, or 14 days, after the likely or confirmed exposure to the coronavirus. In some embodiments, the administration of the one or more TPP Compounds, the one or more ApoA-I mimetic peptides, and/or the one or more Nrf2 agonists occurs for at least 1-10 days after the exposure or likely exposure to the coronavirus. In some embodiments, the administration of the one or more TPP Compounds, the one or more ApoA-I mimetic peptides, and/or the one or more Nrf2 agonists occurs for at least 1-10 days before the exposure or likely exposure to the coronavirus. In some embodiments, the coronavirus is SARS-CoV-2. In some embodiments, one or more Nrf2 agonists, e.g., dimethyl fumarate (DMF), may be administered to the cell or the subject. In some embodiments, the amount of the one or more Nrf2 agonists that is administered to the subject is about 0.02-8.0 mg/kg, about 0.15-8.0 mg/kg, about 4.0-8.0 mg/kg, about 0.01-4.0 mg/kg, about 0.1-4.0 mg/kg, or about 2.0-4.0 mg/kg weight of the subject. In some embodiments, the amount of the one or more Nrf2 agonists that is administered to the subject is about 1-480 mg, about 10-480 mg, about 240-480 mg, about 0.5-240 mg, about 5-240 mg, or about 120-240 mg. In some embodiments, administration of the one or more ApoA-I mimetic peptides and/or the one or more Nrf2 agonists is concurrent with the one or more TPP Compounds. In some embodiments where both one or more TPP Compounds and one or more ApoA-I mimetic peptides are administered (without the administration of an Nrf2 agonist), the ratio of the amount of the one or more TPP Compounds to the amount of the one or more ApoA-I mimetic peptides that is administered ranges from about 0.0001:1 to about 100:1, preferably about 0.1:1 by molecular amount (moles). In some embodiments where one or more TPP Compounds, one or more ApoA-I mimetic peptides, and one or more Nrf2 agonists are administered, the ratio of the amount of the one or more TPP Compounds to the amount of the one or more ApoA-I mimetic peptides that is administered ranges from about 0.001:1 to about 10:1, preferably about 0.01:1, by molecular amount (moles). In some embodiments where one or more TPP Compounds+one or more ApoA-I mimetic peptides+one or more Nrf2 agonists are administered, the relative amounts of the one or more TPP Compounds:the one or more ApoA-I mimetic peptides:the one or more Nrf2 agonists that are administered is about 0.1:1:10 by molecular amount (moles).

In some embodiments, the invention is directed to the use of one or more TPP Compounds alone or in combination with one or more ApoA-I mimetic peptides and/or one or more Nrf2 agonists (1) as an antiviral against a coronavirus; (2) to prevent, inhibit, and/or reduce apoptosis caused by infection by a coronavirus; (3) in the treatment of an infection by a coronavirus or in the manufacture of a medicament for treating the infection; (4) in the treatment of apoptosis and/or inflammation caused by a coronavirus or in the manufacture of a medicament for treating and/or inflammation caused by the coronavirus. In some embodiments, the use of one or more TPP Compounds alone or in combination with one or more ApoA-I mimetic peptides and/or one or more Nrf2 agonists is to prevent, inhibit, reduce, and/or treat (a) an infection by, (b) an inflammatory response caused by, (c) apoptosis caused by, (d) injury to lung tissue caused by, or (e) a disease caused by a coronavirus, such as SARS-CoV-2. In some embodiments, the use of one or more TPP Compounds alone or in combination with one or more ApoA-I mimetic peptides and/or one or more Nrf2 agonists is for the manufacture of a medicament for preventing, inhibiting, reducing, and/or treating (a) an infection by, (b) an inflammatory response caused by, (c) apoptosis caused by, (d) injury to lung tissue caused by, or (e) a disease caused by a coronavirus, such as SARS-CoV-2. In some embodiments, the one or more TPP Compounds is used concurrently with one or more ApoA-I mimetic peptides and/or one or more Nrf2 agonists. In some embodiments, the one or more TPP Compounds is a TPP Hydrocarbon or a TPP Conjugate. In some embodiments, the one or more TPP Compounds is Mito-MES. In some embodiments, the one or more TPP Compounds is dTPP. In some embodiments, dTPP and coenzyme Q10 are provided in the form of a mixture (i.e., instead of a TPP Conjugate, wherein the bioactive moiety is coenzyme Q10). In some embodiments the one or more TPP Compounds and the one or more Nrf2 agonists are provided in the form of a conjugate, i.e., a TPP Conjugate, wherein the bioactive moiety is an Nrf2 agonist. In some embodiments, the one or more TPP Compounds is not Mito-MES, MitoQ™, SkQ1 (Mitotech, S.A.), Elamipretide (Stealth BioTherapeutics), Mito-TEMPO (CAS 1569257-94-8) or a compound disclosed in U.S. Pat. Nos. 8,518,915; 9,192,676; 9,328,130; 9,388,156; US20070161609; US20070225255; US20080161267; US20100168198; US20160200749; US20180305328; US20190248816; US20190330249; US20190374558; WO2005019232; WO2006005759; WO2007046729; WO2008145116; WO2015063553; WO2017106803; and WO2018162581. The one or more ApoA-I mimetic peptides may be 4F, D-4F, reverse D-4F, or 6F. In some embodiments, the amount of the one or more TPP Compounds is about 1-1000 mg, 5-100 mg, 10-80 mg, or 20-40 mg, preferably about 20 mg. In some embodiments, the amount of the one or more TPP Compounds is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. In some embodiments, the amount of the one or more ApoA-I mimetic peptides is about 1-500 mg, about 10-500 mg, about 100-500 mg, about 1-250 mg, about 10-250 mg, or about 100-250 mg. In some embodiments, up to 1000 mg of the one or more TPP Compounds is provided as several divided doses. In some embodiments, the one or more TPP Compounds, the one or more ApoA-I mimetic peptides, and/or the one or more Nrf2 agonists is formulated for oral administration. In some embodiments, the coronavirus is SARS-CoV-2. In some embodiments, one or more Nrf2 agonists, e.g., DMF, is provided in combination with the one or more TPP Compounds and/or the one or more ApoA-I mimetic peptides. In some embodiments, the amount of the one or more Nrf2 agonists is about 1-480 mg, about 10-480 mg, about 240-480 mg, about 0.5-240 mg, about 5-240 mg, or about 120-240 mg. In some embodiments, the amount of the one or more TPP Compounds, the amount of the one or more ApoA-I mimetic peptides, and/or the one or more Nrf2 agonists is provided as a daily dose. In some embodiments where both one or more TPP Compounds and one or more ApoA-I mimetic peptides are provided (without an Nrf2 agonist), the ratio of the amount of the one or more TPP Compounds to the amount of the one or more ApoA-I mimetic peptides ranges from about 0.0001:1 to about 100:1, preferably about 0.1:1 by molecular amount (moles). In some embodiments where one or more TPP Compounds, one or more ApoA-I mimetic peptides, and one or more Nrf2 agonists are provided, the ratio of the amount of the one or more TPP Compounds to the amount of the one or more ApoA-I mimetic peptides ranges from about 0.001:1 to about 10:1, preferably about 0.01:1, by molecular amount (moles). In some embodiments where one or more TPP Compounds+one or more ApoA-I mimetic peptides+one or more Nrf2 agonists are provided, the relative amounts of the one or more TPP Compounds:the one or more ApoA-I mimetic peptides:the one or more Nrf2 agonists is about 0.1:1:10 by molecular amount (moles).

In some embodiments, the invention is directed to a composition comprising, consisting essentially of, or consisting of one or more TPP Compounds in combination with: (a) one or more ApoA-I mimetic peptides, (b) one or more Nrf2 agonists, or (c) one or more ApoA-I mimetic peptides and one or more Nrf2 agonists. In some embodiments, the one or more TPP Compounds is selected from mitoquinone and salts thereof, mitoquinol and salts thereof, SkQ1, Elamipretide, and Mito-TEMPO. In some embodiments, the one or more TPP Compounds is a TPP Hydrocarbon or a TPP Conjugate. In some embodiments, the one or more TPP Compounds is Mito-MES. In some embodiments, the one or more TPP Compounds is dTPP. In some embodiments, dTPP and coenzyme Q10 are provided in the form of a mixture (i.e., instead of a TPP Conjugate, wherein the bioactive moiety is coenzyme Q10). In some embodiments the one or more TPP Compounds and the one or more Nrf2 agonists are provided in the form of a conjugate, i.e., a TPP Conjugate, wherein the bioactive moiety is an Nrf2 agonist. In some embodiments, the one or more TPP Compounds is not Mito-MES, MitoQ™, SkQ1 (Mitotech, S.A.), Elamipretide (Stealth BioTherapeutics), Mito-TEMPO (CAS 1569257-94-8) or a compound disclosed in U.S. Pat. Nos. 8,518,915; 9,192,676; 9,328,130; 9,388,156; US20070161609; US20070225255; US20080161267; 0520100168198; US20160200749; US20180305328; US20190248816; US20190330249; US20190374558; WO2005019232; WO2006005759; WO2007046729; WO2008145116; WO2015063553; WO2017106803; and WO2018162581. In some embodiments, the one or more ApoA-I mimetic peptides is selected from 4F, D-4F, reverse D-4F, and 6F. In some embodiments, the one or more Nrf2 agonists is selected from Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF). In some embodiments, the one or more Nrf2 agonists is DMF. In some embodiments, the composition comprises Mito-MES+4F, D-4F, reverse D-4F, or 6F. In some embodiments, the composition comprises Mito-MES+DMF. In some embodiments, the composition comprises (1) Mito-MES, (2) 4F, D-4F, reverse D-4F, or 6F, and (3) DMF. In some embodiments, the composition comprises about 1-1000 mg, 5-100 mg, 10-80 mg, or 20-40 mg, and preferably about 20 mg, of the one or more TPP Compounds. In some embodiments, the composition comprises about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about or 200 mg of the one or more TPP Compounds. In some embodiments, the composition comprises about 1-500 mg, about 10-500 mg, about 100-500 mg, about 1-250 mg, about 10-250 mg, or about 100-250 mg of the one or more ApoA-I mimetic peptides. In some embodiments, the composition comprises about 1-480 mg, about 10-480 mg, about 240-480 mg, about 0.5-240 mg, about 5-240 mg, or about 120-240 mg of the one or more Nrf2 agonists. In some embodiments, the composition comprises (a) about 1-1000 mg, 5-100 mg, 10-80 mg, or 20-40 mg, and preferably about 20 mg, of the one or more TPP Compounds and (b) about 1-500 mg, about 10-500 mg, about 100-500 mg, about 1-250 mg, about 10-250 mg, or about 100-250 mg of the one or more ApoA-I mimetic peptides. In some embodiments, the composition comprises (a) about 1-1000 mg, 5-100 mg, 10-80 mg, or 20-40 mg, and preferably about 20 mg, of the one or more TPP Compounds, (b) about 1-500 mg, about 10-500 mg, about 100-500 mg, about 1-250 mg, about 10-250 mg, or about 100-250 mg of the one or more ApoA-I mimetic peptides, and (c) about 1-480 mg, about 10-480 mg, about 240-480 mg, about 0.5-240 mg, about 5-240 mg, or about 120-240 mg of the one or more Nrf2 agonists. In some embodiments of compositions comprising one or more TPP Compounds+one or more ApoA-I mimetic peptides (without an Nrf2 agonist), the ratio of the amount of the one or more TPP Compounds to the amount of the one or more ApoA-I mimetic peptides ranges from about 0.0001:1 to about 100:1, preferably about 0.1:1 by molecular amount (moles). In some embodiments of compositions comprising one or more TPP Compounds+one or more ApoA-I mimetic peptides+one or more Nrf2 agonists, the ratio of the amount of the one or more TPP Compounds to the amount of the one or more ApoA-I mimetic peptides ranges from about 0.001:1 to about 10:1, preferably about 0.01:1, by molecular amount (moles). In some embodiments of compositions comprising one or more mitochondrial targeted antioxidants+one or more ApoA-I mimetic peptides+one or more Nrf2 agonists, the relative amounts of the one or more TPP Compounds:the one or more ApoA-I mimetic peptides:the one or more Nrf2 agonists is about 0.1:1:10 by molecular amount (moles).

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1-a-FIG. 1-s show that Mito-MES inhibits SARS-CoV-2 replication and associated apoptosis. Vero cells were treated with Mito-MES (10 nM-10 μM) (72 h) or 5 μM remdesivir (RDV) (49 h) and infected with SARS-CoV-2 (48 h). Viral replication by TCID50-assay (FIG. 1-a-FIG. 1-c), qPCR (FIG. 1-d) or flow cytometry (FIG. 1-e). Cell cytotoxicity was assessed in uninfected cells by the XTT assay (FIG. 1-f). Calu3 cells treated with Mito-MES (72 h) and infected with SARS-CoV-2 (48 h). Viral replication by TCID50-assay (FIG. 1-g-FIG. 1-i), qPCR (FIG. 1-j) or flow cytometry (FIG. 1-k). Cell cytotoxicity was assessed in uninfected cells by the XTT assay (FIG. 1-1). Air-liquid interphase (ALI) airway model of human airway epithelium (HAE) treated with Mito-MES (100 nM) (72 h) and infected with SARS-CoV-2 (48 h). Viral replication was determined by flow cytometry in total airway epithelial cells (FIG. 1-m), in ciliated airway epithelial cells (positive for FOXJ1 protein) (FIG. 1-n). FIG. 1-o shows viral replication determined by flow cytometry in HEK293T cells stably expressing ACE2 (HEK293T-hACE2 cells), treated with 100 nM Mito-MES (72 h) and infected with SARS-CoV-2 (48 h). Cellular apoptosis was accessed based on protein levels of cleaved Caspase 3 and detected by flow cytometry in Vero (FIG. 1-p, FIG. 1-q) and Calu3 (FIG. 1-r, FIG. 1-s) cells treated with Mito-MES (100 nM) (72 h) and infected with SARS-CoV-2 (48 h). Data in (FIG. 1-a-FIG. 1-d, FIG. 1-f-FIG. 1-j, FIG. 1-1) are pooled data from three independent experiments in duplicates and triplicates. Data points represent one biological sample. Data in (FIG. 1-e, FIG. 1-m-FIG. 1-s) are representative of at least two independent experiments. Bars indicate mean±s.e.m. Unless otherwise stated, statistical comparison was done compared to the vehicle control group (Ctrl) by two-tailed Mann-Whitney (*p<0.05, **p<0.01, ***p<0.001).

FIG. 2-a-FIG. 2-f show that Mito-MES inhibits and/or reduces SARS-CoV-2 infection in epithelial cells and associated apoptosis. Vero and Calu3 cells treated with Mito-MES (10-1000 nM) (72 h) vs DMSO vehicle control (Ctrl) and infected with SARS-CoV-2 (48 h). Viral replication (protein levels of SARS-CoV-2 spike protein) and cellular apoptosis (protein levels of cleaved caspase-3) were assessed by immunofluorescence or flow cytometry. Immunofluorescent analysis of Vero E6 and Calu3 cells infected with SARS-CoV-2 (MOI of 0.1) was performed at 48 h post-infection (hpi). Cells were immuno-stained for SARS-CoV-2 spike protein and apoptosis marker cleaved caspase-3 in infected cells treated with DMSO vehicle control and infected cells treated with Mito-MES (250 nM for Vero E6 and 10 nM for Calu3). DAPI was used to stain cell nuclei. FIG. 2-a and FIG. 2-b summarize the results of the immunofluorescence experiments for the median fluorescence intensity (MFI) of SARS-CoV-2 spike S protein per cell (arbitrary units) in Vero (FIG. 2-a) and Calu3 (FIG. 2-b) cells. FIG. 2-c and FIG. 2-d: Flow cytometry was used to determine the percent of Spike S protein⁺ viable cells. Representative data are shown for Vero (FIG. 2-c) and Calu3 (FIG. 2-d) cells treated with vehicle control (Ctrl) vs Mito-MES (250 nM for Vero and 100 nM for Calu3 cells). FIG. 2-e and FIG. 2-f summarize the results of the immunofluorescence experiments for the MFI of cleaved caspase-3 protein per cell (arbitrary units) in Vero (FIG. 2-e) and Calu3 (FIG. 2-f) cells treated with Mito-MES (10-1000 nM) (72 h) and infected with SARS-CoV-2 (48 h). Data in FIG. 2-a, FIG. 2-b, FIG. 2-e, and FIG. 2-f are pooled data from at least two independent experiments in triplicates. Data in FIG. 2-c and FIG. 2-d are representative of at least two independent experiments. Bars indicate mean±s.e.m. Unless otherwise stated, statistical comparison was done compared to the vehicle control group (Ctrl) by two-tailed Mann-Whitney (*p<0.05, **p<0.01, ***p<0.001).

FIG. 3-a-FIG. 3-e show the antiviral activity of Mito-MES against SARS-CoV-2 in epithelial cells depends only partially on its antioxidant activity. Flow cytometry was used to determine the cellular content of Calu3 cells for mitochondrial reactive oxygen species (mito-ROS) based on the median fluorescence intensity (MFI) of the fluorochrome MitoSOX Red and was compared to a negative cell population (fluorescence minus one negative control for staining) (ΔMFI). Results are summarized in FIG. 3-a. Flow cytometry was used to determine the percent of SARS-CoV-2 infected Calu3 cells positive for MitoSOX Red. Results are summarized in FIG. 3-b. Calu3 and Vero cells were treated with DMSO vehicle control (Ctrl) or the mitochondrial antioxidants MitoTEMPO (25-1000 nM) (72 h) or Mito-MES (25-1000 nM) (72 h) and were infected with a fluorescent mNeonGreen SARS-CoV-2 (icSARS-CoV-2-mNG) (MOI 0.3) (48 h). Immunofluorescent analysis of viral replication and cellular oxidative stress was performed at 48 hpi. Data not shown. Cell cytotoxicity was assessed by the XTT assay in uninfected cells treated with MitoTEMPO (0.01-10 μM) (72 h) and the results are summarized in FIG. 3-c. Seahorse XF Analyzer was used to determine mitochondrial respirometry and cellular bioenergetics [oxygen consumption rate (OCR) and extracellular acidification rate (ECAR)] of live Calu3 cells treated with Mito-MES (25-1000 nM) or vehicle control (ctrl) (72 h) and infected with SARS-CoV-2 (MOI 0.1) (48 h). Data not shown. Calu3 cells were treated with the positively charged lipophilic cation (1-Decyl)triphenylphosphonium (dTPP) bromide that enhances entry of Mito-MES inside mitochondria (10-1000 nM) (72 h) and infected with SARS-CoV-2 (48 h). Viral replication was determined by TCID50-assay (FIG. 3-d), cell cytotoxicity was assessed by the XTT assay in uninfected cells treated with dTPP (0.01-10 μM) (72 h) (FIG. 3-e). Results are pooled data from three independent experiments in duplicates and triplicates. Data-points represent one biological sample. Bars indicate mean±s.e.m. Unless otherwise stated, statistical comparison was done compared to the vehicle control group (Ctrl) by two-tailed Mann-Whitney (*p<0.05, **p<0.01, ***p<0.001).

FIG. 4-a-FIG. 4-g show the impact of Mito-MES on interferon host immune responses in SARS-CoV-2 infected epithelial cells. Protein levels of secreted interferons [beta: IFN-β (FIG. 4-a), lambda: IFN-λ (FIG. 4-b)] were assessed by ELISA in Calu3 cells treated with Mito-MES (100-1000 nM) or vehicle control (ctrl) (72 h) and infected with SARS-CoV-2 (48 h). Protein levels of cellular interferons [IFN-β (FIG. 4-c), lambda: IFN-λ (FIG. 4-d)] were assessed by ELISA in in protein lysates of Calu3 cells treated with Mito-MES (100-1000 nM) or vehicle control (ctrl) (72 h) and infected with SARS-CoV-2 (48 h). Flow cytometry and immunofluorescence were used to assess protein levels (fluorescence intensity of target protein) of host proteins important for the crosstalk between mitochondria and interferon host immune responses in cells infected with SARS-CoV-2 (48 h) and in total (infected and uninfected) single viable Calu3 cells treated with Mito-MES (100 or 1000 nM) (72 h) or vehicle control (ctrl). Protein levels of the Mitochondrial antiviral-signaling protein (MAVS) were accessed by flow cytometry in total and SARS-CoV-2 infected single viable Calu3 cells. Data not shown. Protein levels of the mitochondrial outer membrane 70 (TOM70) were accessed by flow cytometry in total and SARS-CoV-2 infected single viable Calu3 cells. Data not shown. Immunofluorescent of protein levels of TOM70 in Calu3 cells was accessed at 48 hpi. The results of the immunofluorescence experiments for TOM70 protein [median fluorescence intensity (MFI) per cell, arbitrary units] are summarized in FIG. 4-e. Protein levels of the stimulator of interferon genes (STING) were assessed by flow cytometry in total and SARS-CoV-2 infected single viable Calu3 cells. Data not shown. Protein levels [MFI (FIG. 4-f), or % of cells positive for target protein (FIG. 4-g)] of the MX Dynamin Like GTPase 1 (MX1) were assessed by flow cytometry in total and SARS-CoV-2 infected single viable Calu3 cells. Data from at least three independent experiments. Bars indicate mean±s.e.m. Data-points represent one biological sample. Unless otherwise stated, statistical comparison was done compared to the vehicle control group (Ctrl) by two-tailed Mann-Whitney (*p<0.05, **p<0.01, ***p<0.001).

FIG. 5-a-FIG. 5-i show the antiviral and anti-inflammatory activity of Mito-MES against SARS-CoV-2 in epithelial cells is mediated through the Nrf2 pathway. Calu3 cells treated with Mito-MES (10-1000 nM, 72 h) and/or the Nrf2 agonist Dimethyl fumarate (DMF) (10, 100 μM, 16-24 h before infection) and/or the Nrf2 inhibitor brusatol (0.25 μM, 16-24 h before infection) and infected with SARS-CoV-2 (48 h) (MOI 0.1). FIG. 5-a summarizes the results of the viral replication by TCID50-assay at 48 hpi in Calu3 cells treated with Mito-MES (10-1000 nM, 72 h) and/or DMF (10, 100 μM, 16-24 h). FIG. 5-b provides the % cytopathogenic effect at 48 hpi in untreated Calu3 cells (Ctrl, dashed line) Calu3 cells treated with Mito-MES (100 nM, 72 h, solid line), and 100 nM Mito-MES 10 μM DMF (16-24 h, dotted line). FIG. 5-c provides the % inhibition with Mito-MES alone vs Mito-MES in combination with 10 μM DMF at 48 hpi in Calu3 cells treated with Mito-MES (1-1000 nM, 72 h, solid line, IC50=0.030 μM) and/or DMF (10 μM, 16-24 h, IC50 0.009 μM). FIG. 5-d summarizes the viral replication by TCID50-assay at 48 hpi in Calu3 cells treated with Mito-MES (100, 1000 nM, 72 h) and/or brusatol (0.25 μM, 16-24 h). Cell cytotoxicity was assessed by the XTT assay in uninfected Calu3 cells treated with DMF or brusatol (0.25 μM) for 24 h and the results are summarized in FIG. 5-e. Viral replication (% of cells positive for the Nucleocapsid N SARS-CoV-2 protein) was determined by flow cytometry at 48 hpi in Calu3 cells treated with Mito-MES (100 nM, 72 h) and/or DMF (10, 100 μM, 16-24 h). Data not shown. Viral replication by flow cytometry was determined at 48 hpi in Calu3 cells treated with Mito-MES (1000 nM, 72 h) and/or brusatol (0.25 μM, 16-24 h). Data not shown. Viral replication and nuclear factor erythroid 2 [NF-E2]-related factor 2 [Nrf2]) protein levels were determined by flow cytometry at 48 hpi in Calu3 cells treated with Mito-MES (100 nM, 72 h) and/or DMF (10, 100 μM, 16-24 h). Data not shown. Viral replication and protein levels of Nrf2 by flow cytometry were determined at 48 hpi in Calu3 cells treated with Mito-MES (1000 nM, 72 h) and/or brusatol (0.25 μM, 16-24 h). Data not shown. Secretion of cytokines by SARS-CoV-2 infected Calu3 cells in cell culture supernatants collected at 48 h post SARS-CoV-2 infection (MOI 0.1) in Calu3 cells treated with Mito-MES (100, 1000 nM, 72 h) and/or DMF (10, 100 μM, 16-24 h) and/or brusatol (0.25 μM, 16-24 h). The compared groups were uninfected cells (Mock), cells infected with SARS-CoV-2 treated with DMSO vehicle control (Ctrl), and cells infected with SARS-CoV-2 treated with Mito-MES (100 nM, 1000 nM), DMF or brusatol. ELISA was used to measure IL-6 (ng/ml) secreted by Calu3 cells 48 h post SARS-CoV-2 infection in cells treated as shown in FIG. 5-f. Luminex immunoassay was used to measure IL-1β, IL-8, IL-10, and TNF-α secreted by Calu3 cells in cell culture supernatants collected at 48 h post SARS-CoV-2 infection. The results are summarized in FIG. 5-g (in each set of bars, the first is uninfected cells, the second is SARS-CoV-2 infected cells, and the third bar is SARS-CoV-2 infected cells treated with Mito-MES). The mean value of each measurement (protein levels in cell culture supernatants in pg/ml) was normalized by the mean value of each measurement in the vehicle group and expressed as fold to the mean of the vehicle control group. ELISA was used to measure IL-6 (ng/ml) secreted by Calu3 cells 48 h post SARS-CoV-2 infection in cells treated with Mito-MES and/or DMF vs vehicle control. In FIG. 5-h, 1=Mock, 2=Ctrl, 3=10 μM DMF, 4=100 μM DMF, 5=100 nM Mito-MES, 6=100 nM Mito-MES+10 μM DMF, 7=100 nM Mito-MES+100 μM DMF, 8=1000 nM Mito-MES, 9=1000 nM Mito-MES+10 μM DMF, 10=1000 nM Mito-MES+100 DMF. ELISA was used to measure IL-6 (ng/ml) secreted by Calu3 cells 48 h post SARS-CoV-2 infection in cells treated as shown in FIG. 5-i with Mito-MES and/or brusatol vs vehicle control. Except for FIG. 5-b and FIG. 5-c (which provide representative data from at least three independent experiments), the results shown are pooled data from three independent experiments in duplicates and triplicates. Data-points represent one biological sample. Bars indicate mean±s.e.m. Unless otherwise stated, statistical comparison was done compared to the vehicle control group (Ctrl) by two-tailed Mann-Whitney (*p<0.05, **p<0.01, ***p<0.001).

FIG. 6-a show the impact of Mito-MES on cytokines secreted by Vero cells infected with SARS-CoV-2. Vero cells were treated with 10-1000 nM Mito-MES or vehicle control (72 h) and infected with SARS-CoV-2 (MOI 0.1) (48 h). Secreted IL-6 was determined by ELISA in cell culture supernatants collected 48 hpi.

FIG. 7-a and FIG. 7-b show that Mito-MES inhibits and/or reduces SARS-CoV-2 infection in vivo. K18-hACE2 transgenic mice were infected with SARS-CoV-2 (10,000 PFU per mouse) intranasally and treated with Mito-MES (4 mg/kg per day intraperitoneally) (n=10) vs vehicle control (Ctrl) (saline 1% DMSO) (n=5 mice). Five mice (n=5) were treated with Mito-MES for 48 h before SARS-COV-2 infection and throughout the infection for 3 days (5 days in total) until mice were sacrificed 72 hpi. Five mice (n=5) were treated with Mito-MES 8 h after the infection for 3 days (3 days in total) until mice were sacrificed 72 hpi. The left lung was harvested from all mice, tissue was homogenized in mechanical dissociator and the supernatant was cryopreserved until it was used for viral titer. Viral titer was determined by two independent methods based on cytopathic effect (CPE) effect in Vero-E6 cells and based on an ELISA immunoassay that detected the presence of SARS-CoV-2 Spike S protein in Vero-E6 cells infected with lung supernatants from infected mice. FIG. 7-a summarizes the results of the viral replication by TCID50-assay based on CPE method. FIG. 7-b summarizes the results of the viral replication by TCID50-assay based on ELISA assay. Bars indicate mean±s.e.m. Unless otherwise stated, statistical comparison was done compared to the vehicle control group (Ctrl) by two-tailed Mann-Whitney (*p<0.05, **p<0.01, ***p<0.001).

FIG. 8-a show the timeline of exposure, symptoms and diagnostic tests in household. Two parents [father 40 years, A1 with history of chronic myelogenous leukemia (CML) and healthy mother 39 years, A2] were notified on Dec. 5, 2020 [defined as day 5 (D5)], that the schoolteacher (T1) of their 5-year-old son (C1) tested positive for SARS-CoV-2 and developed symptoms of Covid-19. C1 had 6-h exposure to T1 on November 30 (with masks) (D0). The family had no other exposure to any other person between November 20-30 (D-10 to D0) given Thanksgiving Day on November 26 (D-4). Several children and parents from the same school class were infected with Covid-19 within the first 2 weeks of December. C1 developed fever (100.4° F.; 38° C.) and fatigue 2.5 days after exposure (D0) that lasted 3 days (D2-D5) and remained asymptomatic since then. The second child (female 3 years, C2) was asymptomatic until D12 when she developed fever (100.4° F.; 38° C.), fatigue and coryza on D12-D14. A1 and A2 started Mito-MES on D4. C2 had a positive SARS-CoV-2 PCR on nasopharyngeal (NP) swab taken on D7 while a SARS-CoV-2 PCR NP swab on D30 was indeterminate. A1, A2, C1 and C2 had fifteen (in total) negative NP SARS-CoV-2 PCR and each parent had 3 negative SARS-CoV-2 IgG serology over a period of 6 weeks (last NP PCR was on D42 and last SARS-CoV-2 IgG serology was on D45). The well-defined epidemiologic history around a major holiday and the timing (3-7 days) of development of symptoms in both children, confirmed that they both had Covid-19.

FIG. 9-a-FIG. 9-e show that 4F inhibits and/or reduces SARS-CoV-2 infection in epithelial cells. Vero E6 and Calu3 cells were infected with SARS-CoV-2 (MOI 0.1) and treated with media alone (vehicle), remdesivir (49 h) or 4F (1-100 μM) (72 h) as in methods. qRT-PCR analysis of Vero E6 (FIG. 9-a) and Calu3 (FIG. 9-b) cells 48 hpi. The graph depicts the relative amount of SARS-CoV-2 Spike S normalized to human GADPH. Relative viral quantification was done compare to the positive control (infected cells treated with vehicle). Viral titers were determined in supernatants at 48 hpi in Vero E6 cells (FIG. 9-c) and Calu3 cells (FIG. 9-d). Confluent Calu3 cells treated with vehicle (media) or 4F (10 μM) were fixed 48 hpi followed by processing for immunostaining with the SARS-CoV-2 Spike S antibody and DAPI. The percentage of SARS-CoV-2-infected cells and the median fluorescence intensity of SARS-CoV-2 Spike S protein per cell (MFI in arbitrary units) were determined. FIG. 9-e summarizes the immunofluorescence data (% of SARS-CoV-2⁺ cells and SARS-CoV-2 Spike S MFI in cells fold to vehicle control (media). Data represent the mean±SEM, representing three independent experiments performed in at least two technical replicates. The Kruskal-Wallis statistical test was used to compare 3 groups and the Mann-Whitney was used to compare each group relative to the vehicle control and the p value for this comparison is shown above each column (*p<0.05, **p<0.01, ***p<0.001).

FIG. 10-a-FIG. 10-c show that 4F inhibits and/or reduces apoptosis and inflammatory responses associated with SARS-CoV-2 infection in lung epithelial cells. Vero E6 and Calu3 cells were uninfected (mock) or infected with SARS-CoV-2 (MOI 0.1) and were treated with media alone (vehicle), or 4F (10 μM) (72 h) as described in the detailed experiments. Confluent cells were fixed 48 h hpi followed by processing for flow cytometry. Flow cytometry was used in Vero E6 cells to determine the percent of viable cells positive for cleaved caspase 3 and the median fluorescence intensity (MFI) of cleaved caspase 3 compared to a negative cell population (data not shown). Flow cytometry was used in Calu3 cells to determine the percent of viable cells positive for cleaved caspase 3 and the MFI of cleaved caspase 3 compared to a negative cell population (data not shown). Representative data from three independent experiments are shown. ELISA was used to determine protein levels of IL-6 (ng/ml) secreted by Calu3 (FIG. 10-a) and Vero-E6 (FIG. 10-b) cells 48 h post SARS-CoV-2 infection. The compared groups were uninfected cells (mock, light grey), cells infected with SARS-CoV-2 (SARS-CoV-2⁺, red), and cells infected with SARS-CoV-2 treated with 4F (light blue). Data represent the mean±standard error of means (SEM), representing three independent experiments conducted at least in triplicate. The Mann-Whitney was used to compare each group relative to the vehicle control and the p value for this comparison is shown above each column (***p<0.001). Luminex immunoassays were used to determine protein levels of IL-1β, IL-8, IL-10, TNF-α (pg/ml) secreted by Calu3 cells 48 h post SARS-CoV-2 infection. The results are summarized in FIG. 10-c. The mean value of each measurement (protein levels in cell culture supernatants in pg/ml) was normalized by the mean value of each measurement in the vehicle group and expressed as fold to the mean of the vehicle control group. Data represent the mean±SEM, representing three independent experiments conducted at least in triplicate. The Kruskal-Wallis statistical test was used to compare 3 groups and the Mann-Whitney was used to compare each group relative to the vehicle control. The p value for this comparison is shown above each column (*p<0.05, **p<0.01, ***p<0.001).

FIG. 11-a shows that the antiviral activity of Mito-MES against SARS-CoV-2 in epithelial cells is synergistically enhanced when combined with the ApoA-I mimetic peptide 4F and further synergy is provided by the addition of the Nrf2 agonist dimethyl fumarate (DMF). Calu3 cells treated with 10 nM Mito-MES (72 h) and/or 10 μM DMF (16-24 h) and/or 100 nM or 1000 nM 4F (72 h) and infected with SARS-CoV-2 (48 h) (MOI 0.1). Viral replication was determined by TCID50-assay at 48 hpi. Data are pooled data from three independent experiments in triplicates. Data-points represent one biological sample. Bars indicate mean±s.e.m. Statistical comparisons compared to the vehicle control group (Ctrl) was done by two-tailed Mann-Whitney and is shown in blue (*p<0.05, **p<0.01, ***p<0.001). Statistical comparisons between two groups were done by two-tailed Mann-Whitney and is shown in black (*p<0.05, **p<0.01, ***p<0.001).

FIG. 12-a-FIG. 12-d: Mitoquinone mesylate strongly inhibits SARS-CoV-2 replication in multiple types of epithelial cells. FIG. 12-a-FIG. 12-d). Vero E6 cells [(FIG. 12-a) and (FIG. 12-b)], Calu3 cells [(FIG. 12-c), (FIG. 12-d)] were treated with indicated doses of remdesivir (RDV) [(FIG. 12-b) (FIG. 12-d)] or mitoquinone mesylate (Mito-MES) [(FIG. 12-a), (FIG. 12-c)]. IC50, 50% cytotoxic concentration (CC50) values are indicated. Viral replication at 48 hpi by TCID50-assay (FIG. 12-a-FIG. 12-d). Cell cytotoxicity was assessed in uninfected cells at 48 hours by the XTT assay. Viral replication by flow cytometry in human airway epithelial cells was examined (Control 20.0%, 1 μM Mito-MES 0.4%). For all experiments cells were infected with SARS-CoV-2 at an MOI of 0.1 and were treated with drugs for 2 hours before infection and throughout the experiment unless stated otherwise. For “Entry” treatment, the drugs were added to the cells for 2 h before infection and at 2 hpi, the virus-drug supernatant was replaced with culture medium. In all panels, data are means±SEM of at least three independent experiments.

FIG. 13-a-FIG. 13-j: Mito-MES has antiviral activity against SARS-CoV-2 variants and coronaviruses that partially depends on its lipophilic and antioxidant components. Calu3 (FIG. 13-a, FIG. 13-b), human airway epithelial (HAE) cells (FIG. 13-c) and 17CL-1 cells were infected with SARS-CoV-2 Beta (FIG. 13-a) or Delta variant (FIG. 13-b, FIG. 13-c) or mouse hepatitis virus (MHV) (FIG. 13-d) and treated with vehicle control (Ctrl) or mitoquinone mesylate (Mito-MES) as shown. Viral replication by TCID50-assay and cytotoxicity (XTT assay) at 48 hpi (FIG. 13-a-FIG. 13-d). IC50, 50% cytotoxic concentration (CC50) values are indicated (FIG. 13-a, FIG. 13-b, FIG. 13-d). (FIG. 13-e, FIG. 13-f, FIG. 13-g) Time-of-addition experiment of remdesivir (RDV), Mito-MES (FIG. 13-e, FIG. 13-f), dodecyltriphenylphosphonium (dTPP) and coenzyme Q10 (coQ10) (FIG. 13-g) in Calu3 cells (FIG. 13-e), hACE2 HEK293T cells (FIG. 13-f, FIG. 13-g) at entry, post-viral entry and throughout 24 hpi (full time). Levels of intracellular SARS-CoV-2 Nucleocapsid protein were determined by ELISA. (FIG. 13-h, FIG. 13-i) Viral replication at 48 hpi by TCID50-assay in HAE cells treated with dTPP (H) or CoQ10 (FIG. 13-i) as shown. FIG. 13-j: Calu3 cells were treated as shown with Ctrl or Mito-TEMPO (MT) and were infected with fluorescent SARS-CoV-2 (MOI 0.3). For all experiments cells were infected with SARS-CoV-2 at an MOI of 0.1 and were treated with drugs for at least 2 hours before infection and for 48 hrs unless stated otherwise. In all panels, data are representative or means±SEM of at least two experiments. Statistical comparison was done compared to Ctrl by Mann-Whitney (***p<0.001). For FIG. 13-c, FIG. 13-h, and FIG. 13-i, in each set of bars, the first bar is Viral Titer and the second bar is Cytotoxicity. For FIG. 13-e, FIG. 13-f, and FIG. 13-g, in each set of bars, the first bar is Full time, the middle bar is Entry, and the third bar is Post-entry.

FIG. 14-a: The antiviral activity of Mito-MES against SARS-CoV-2 in epithelial cells is mediated through the Nrf2 pathway. Calu3 cells infected with SARS-CoV-2 (48 h) (MOI 0.1) and treated with Mito-MES (10-1000 nM, 72 h) and/or the nuclear factor erythroid 2-related factor 2 (Nrf2) agonist Dimethyl fumarate (DMF) (10, 100 μM, 16-24 hrs) and/or the Nrf2 inhibitor brusatol (BRU) (0.25 μM, 16-24 hrs) or DMSO vehicle control (Ctrl). Protein levels of cleaved caspase-3 in Calu3 cells as assessed by immunofluorescence. Summary (means±SEM) or representative data of at least three experiments in duplicates and triplicates are shown. Data-points represent one sample. Statistical comparison was done compared to the Ctrl by Mann-Whitney (*p<0.05, **p<0.01, ***p<0.001).

FIG. 15-a-FIG. 15-h: Mito-MES inhibits SARS-CoV-2 replication in mouse model of SARS-CoV-2 infection. hACE2 K18 mice were infected intranasally with SARS-CoV-2 (10,000 PFU/mouse) and lung tissue was harvested 72 hpi. FIG. 15-a: Study design for SARS-CoV-2 (FIG. 7-a and FIG. 7-b). Mice were infected with wild type (WT) SARS-CoV-2 and treated with vehicle control (saline 10% DMSO; Ctrl) (n=5) or mitoquinone mesylate (Mito-MES) 4 mg/kg/day (n=10) given intraperitoneally 24 hours before the infection (hbi) (n=5) or 8 hpi (n=5). FIG. 15-b: Study design for SARS-CoV-2 Delta variant. Mice were infected with SARS-CoV-2 Delta variant and treated through gavage with Ctrl (n=10) or Mito-MES 20 mg/kg/day (n=10) 24 hbi. FIG. 15-c: Supernatants from lung homogenates were used for viral titer based on cytopathic effect; measurement of intracellular SARS-CoV-2 nucleocapsid protein (NP) by ELISA in Vero-E6 cells. FIG. 15-d: Measurement of SARS-CoV-2 NP by ELISA in lung homogenates. FIG. 15-e-FIG. 15-h: Assessment of SARS-CoV-2 NP in EPCAM(+) lung cells (FIG. 15-e-FIG. 15-f) and FOXJ1(+) ciliated cells (FIG. 15-g, FIG. 15-h) by flow cytometry. Summary (mean±SEM) data from experiments in triplicates. Datapoints represent one biological sample. Unless otherwise stated, statistical comparison was done compared to the Ctrl by Mann-Whitney ***p<0.001, **p<0.01, *p<0.05.

FIG. 16-a-FIG. 16-c: Mito-MES has anti-inflammatory activity in SARS-CoV-2 infected epithelial cells. FIG. 16-a: Large airway epithelial cells cultured in air-liquid interface (ALI) were treated with DMSO vehicle control (Ctrl) or Mito-MES (1000 nM) and were infected with SARS-CoV-2 Delta variant (MOI 1). Cytokines were measured by Luminex immunoassay at 48 hpi. In each grouping the first bars are uninfected cells (Mock), the second bars are Ctrl, and the third bars are Mito-MES. FIG. 16-b: Like FIG. 16-a, small distal airway epithelial airway epithelial cells cultured in ALI were assayed. In each grouping the first bars are uninfected cells (Mock), the second bars are Ctrl, and the third bars are Mito-MES. FIG. 16-c: Small distal airway epithelial airway epithelial cells cultured in ALI were treated with Mito-MES, dTPP, and CoQ10 as shown. Data are pooled data (mean±SEM) from three experiments in duplicates and triplicates. Data-points represent one biological sample. Unless otherwise stated, statistical comparison was done compared to the Ctrl by two-tailed Mann-Whitney (*p<0.05, **p<0.01).

FIG. 17-a-FIG. 17-g: Mito-MES inhibits SARS-CoV-2 associated inflammation and lung damage in mouse model of SARS-CoV-2 infection. hACE2 K18 mice were infected intranasally with SARS-CoV-2 wild type (WT) or Beta variant (10,000 PFU/mouse), treated with mitoquinone mesylate (Mito-MES) 4 mg/kg/day or vehicle control (Ctrl) and lung was harvested on day 3 (FIG. 15-a) or 5-7 (cohort B) post-infection. (FIG. 17-a) Study design of cohort B. (FIG. 17-b-FIG. 17-e) Supernatants from lung homogenates of mice from cohort A (FIG. 17-b, FIG. 17-c) or B (FIG. 17-d, FIG. 17-e) were used for measurement of cytokines using Luminex immunoassay. (FIG. 17-f) Flow cytometry assessed frequency of CD45+ immune cells in lungs from mice in cohort A. FIG. 17-g: Harvested lungs from mice in cohort B were paraffin-embedded and 5 μm sections were stained for Masson's Trichrome. Histopathology score was determined according to Methods. Summary (mean±SEM) data from experiments in triplicates. Datapoints represent one biological sample. Unless otherwise stated, statistical comparison was done compared to the Ctrl by Mann-Whitney ***p<0.001, **p<0.01, *p<0.05.

FIG. 18-a-FIG. 18-b: Mito-MES inhibits SARS-CoV-2 replication in lung cells in mouse model of SARS-CoV-2 infection. hACE2 K18 mice (n=15) were infected with SARS-CoV-2 and treated intraperitoneally with Mito-MES (n=10) or vehicle control (Ctrl) (n=5). Lung tissue was harvested on day 3 post-infection. Assessment of SARS-CoV-2 N protein by flow cytometry in EPCAM(+) lung epithelial cells (FIG. 18-a) and FOXJ1(+)EPCAM(+) ciliated cells (FIG. 18-b). Summary (means±SEM) or representative data of at least three experiments in duplicates and triplicates are shown. Data-points represent one sample. Unless otherwise shown, statistical comparison was done compared to the Ctrl by Mann-Whitney (*p<0.05, **p<0.01).

FIG. 19-a-FIG. 19-e: Mito-MES inhibits SARS-CoV-2 associated inflammation and lung cell apoptosis in mouse model of SARS-CoV-2 infection. hACE2 K18 mice were infected intranasally with SARS-CoV-2 wild type (WT) or Beta variant (10,000 PFU/mouse), treated with mitoquinone mesylate (Mito-MES) 4 mg/kg/day or vehicle control (Ctrl) and lung was harvested on day 3 (cohort A) or day 5-7 (cohort B) post-infection, as shown in FIG. 17-a. Supernatants from lung homogenates of mice from cohort A (FIG. 19-a, FIG. 19-b) and B (FIG. 19-c, FIG. 19-d) were used for measurement of cytokines using Luminex immunoassay. FIG. 19-e: Flow cytometry was used to assess frequency of CD45+ immune cells in lung cell suspension from mice infected with SARS-CoV-2 Beta variant. Summary data (mean±SEM) from experiments in triplicates. Datapoints represent one biological sample. Unless otherwise stated, statistical comparison was done compared to the Ctrl by two-tailed Mann-Whitney (*p<0.05, **p<0.01).

FIG. 20-a shows the anatomy of TPP Conjugates comprising a bioactive moiety conjugated to the TPP moiety via a hydrocarbon linker (gray).

In the descriptions above, the time indicated in parentheticals indicates the total treatment or incubation time. For example, “SARS-CoV-2 (48 h)” indicates that the given cells were incubated with SARS-CoV-2 for 48 hours. Except for DMF, drug treatments continued throughout the viral infection and incubation period thereafter. Thus, “10 nM Mito-MES (72 h)” in combination with “SARS-CoV-2 (48 h)” indicates that the given cells were treated with 10 nM Mito-MES 24 hours before contact with SARS-CoV-2 and the treatment continued through the 48-hour incubation with SARS-CoV-2; and “DMF (16-24 h)” indicates a 16-24 h pretreatment with DMF and the pretreatment with DMF ended upon viral infection.

### DETAILED DESCRIPTION OF THE INVENTION

As disclosed herein, TPP Compounds, mitochondrial targeted antioxidants, such as Mito-MES, and/or ApoA-I mimetic peptides such as 4F, may be used to inhibit or reduce infection by a coronavirus, e.g., SARS-CoV-2, and/or treat a coronavirus disease, e.g., COVID-19, in a subject.

### I. Mito-MES Exhibits Antiviral, Antiapoptotic, and Anti-Inflammatory Activity Against SARS-CoV-2

As shown herein, the mitochondrial antioxidant Mito-MES exhibits potent antiviral activity against SARS-CoV-2 in kidney and lung epithelial cells and lung air-liquid interface cultures. The antiviral effect of Mito-MES is partially mediated through its antioxidant effect, the Nrf2 pathway, and its hydrophobic TPP+ moiety. In lung epithelial cells, Mito-MES increases the protein levels of TOM70 and MX1 that interact with mitochondria and induce antiviral host responses. Mito-MES also exhibits antiapoptotic and anti-inflammatory activity in lung epithelial cells infected with SARS-CoV-2.

### Mito-MES Exhibits Antiviral Activity Against SARS-CoV-2

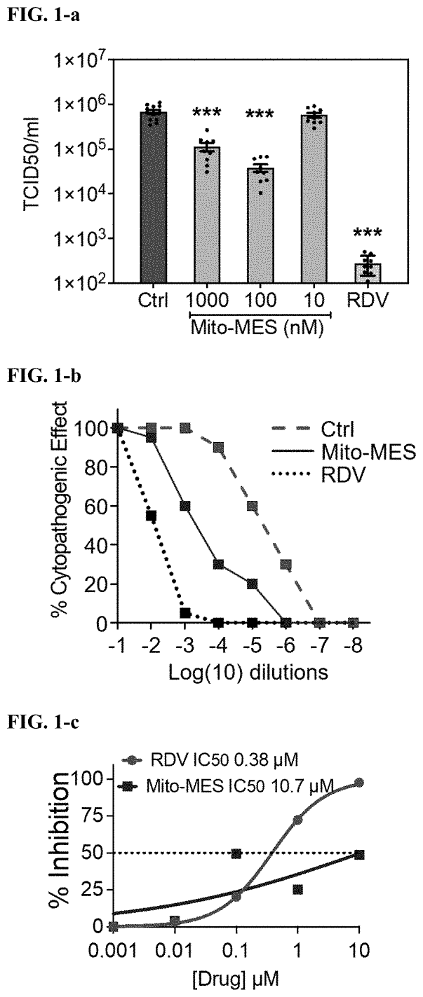

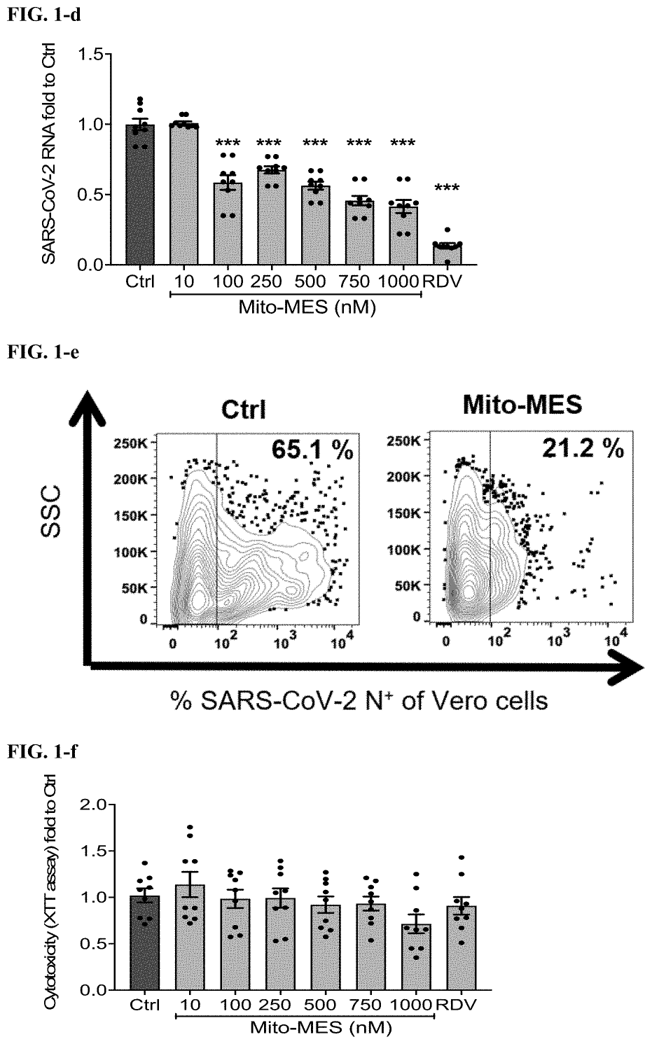

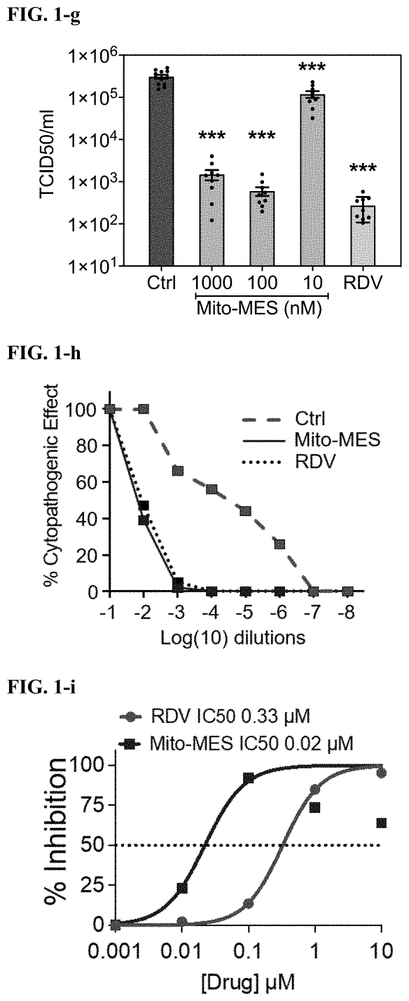

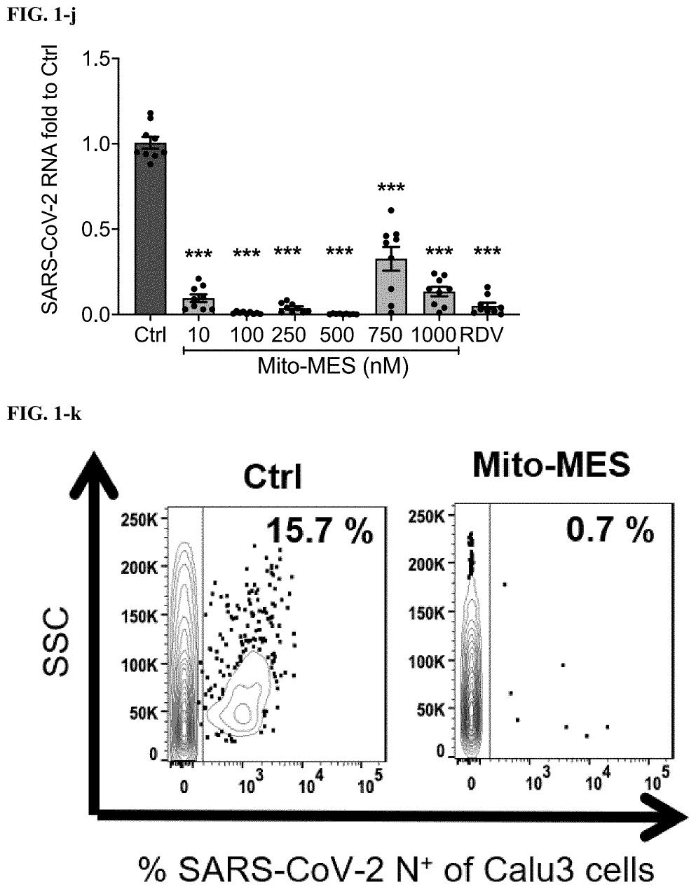

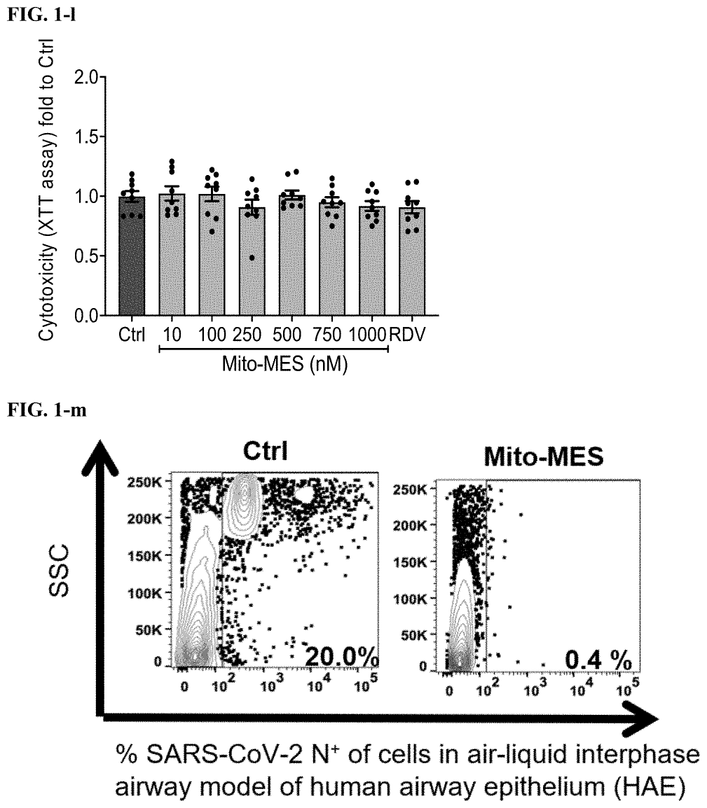

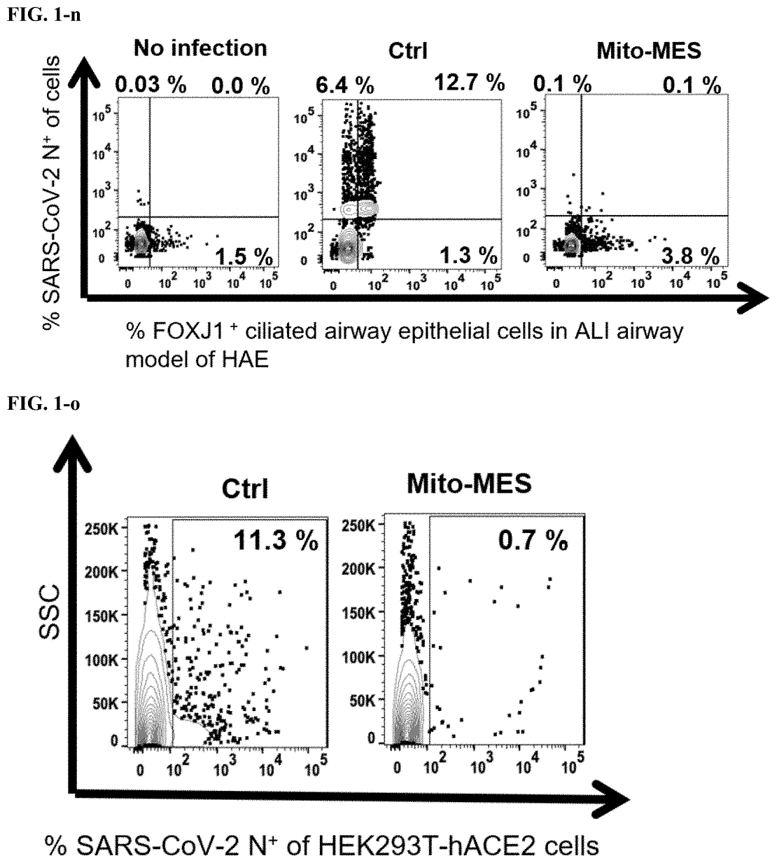

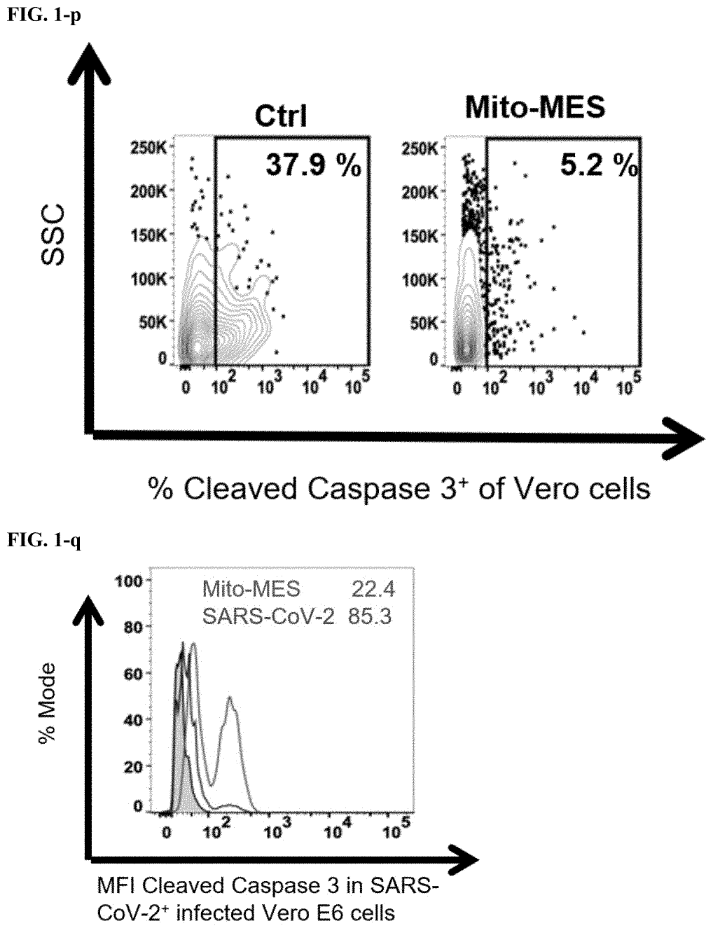

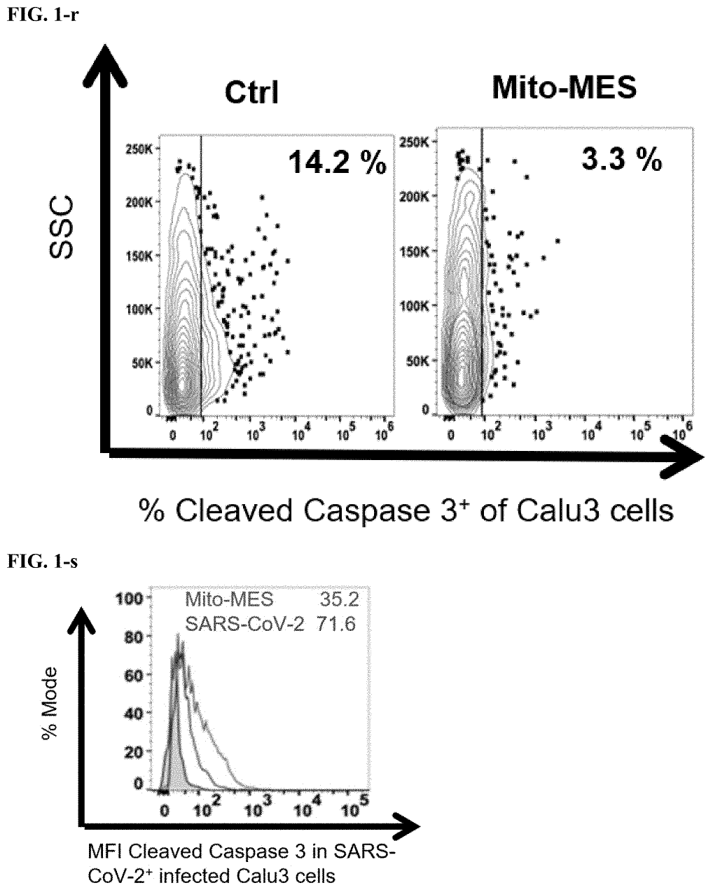

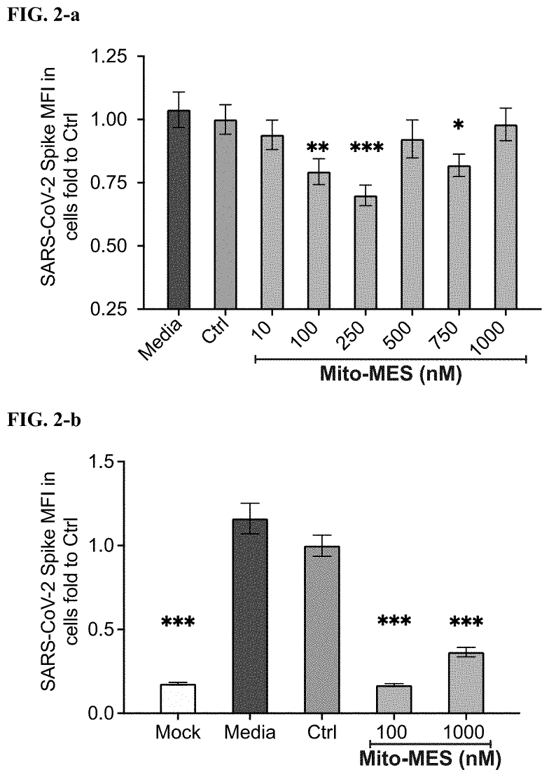

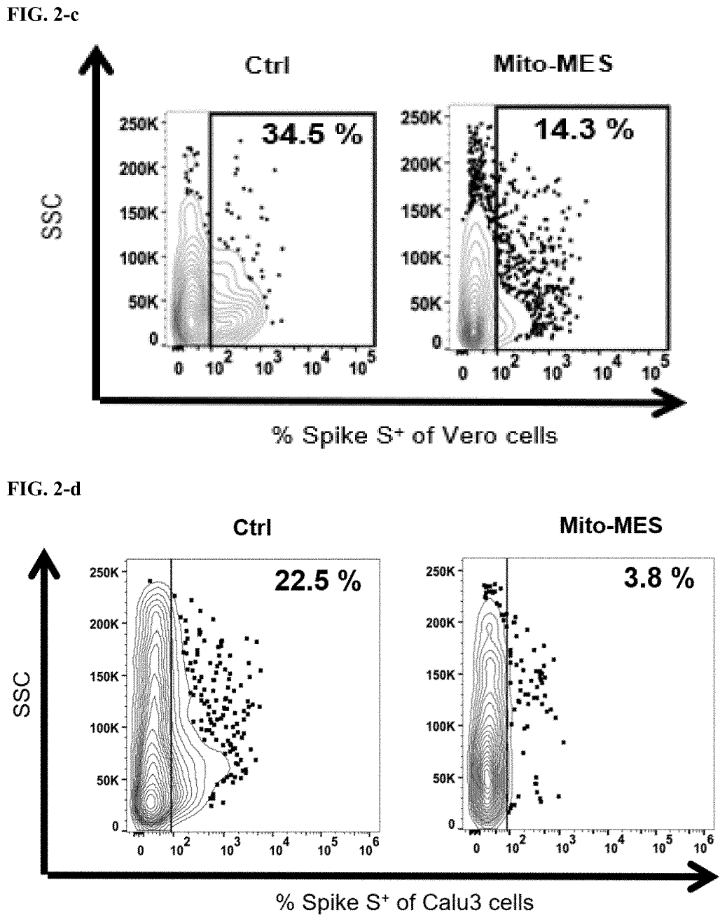

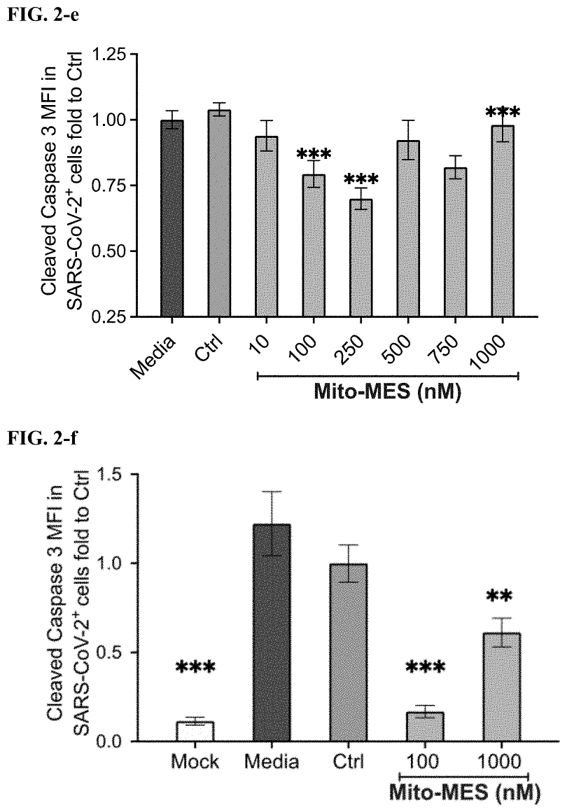

Using a clinical isolate of SARS-CoV-2, virus replication was determined in interferon I (IFN-I)-deficient African green monkey kidney epithelial Vero E6 cells (Vero) and interferon competent human Calu3 lung epithelial cells (Calu3), exposed to a low multiplicity of infection (MOI 0.1) (FIG. 1-a-FIG. 1-1; FIG. 2-a-FIG. 2-d). Treatment of Vero cells with 10-1000 nM Mito-MES before infection with SARS-CoV-2 resulted in reduction in subsequent release of progeny SARS-CoV-2 virus particles to the cell supernatant measured by TCID50 assay to quantify virus by dilution of virus-induced cytopathogenic effects (FIG. 1-a, FIG. 1-b). The antiviral activity was modest compared to 5 μM remdesivir (RDV) that was used as a positive control for antiviral activity against SARS-CoV-2 (FIG. 1-a-FIG. 1-c). Using immunofluorescence, it was found that Mito-MES exhibited modest antiviral activity in Vero cells (FIG. 2-a). These results were confirmed by qPCR that assessed viral genomic RNA in cell lysates (FIG. 1-d).

Flow cytometry was used to characterize the cellular protein levels of the Spike S and the Nucleocapcid N proteins of SARS-CoV-2 in infected cells. It was found that, at the single cell level in viable cells, Mito-MES reduced the percent of infected cells that were positive for N protein (FIG. 1-e) and Spike S protein (FIG. 2-c). There was no observed cytotoxicity associated with Mito-MES, as determined by XTT cell cytotoxicity assay, in similarly treated uninfected cultures across the dose range (10-1000 nM) (FIG. 1-f).

SARS-CoV-2 replication was also evaluated in IFN-I responsive Calu3 cells. Calu3 cells were found to be less-permissive to SARS-CoV-2 replication compared to Vero cells (FIG. 1-a, FIG. 1-e, FIG. 1-g, FIG. 1-k; FIG. 2-a-FIG. 2-d). Despite lower SARS-CoV-2 infection of Calu3 cells, 100 nM and 1000 nM Mito-MES treatment still reduced release of progeny virus by >2-logs and similarly to 5 μM remdesivir based on TCID50 analysis of cell supernatants (FIG. 1-g, FIG. 1-h). Notably, the IC50 of Mito-MES was 0.02 μM compared to 0.33 μM of remdesivir (FIG. 1-i). Both immunofluorescence (FIG. 2-b) and PCR (FIG. 1-j) showed that Mito-MES exhibits potent antiviral activity against SARS-CoV-2 in Calu3 cells at concentrations as low as 10 nM. Flow cytometry experiments further confirmed at the single cell and protein level that Mito-MES inhibits or reduces the percent of SARS-CoV-2 infected Calu3 cells (FIG. 1-k, FIG. 1-f).

The antiviral effect towards SARS-CoV-2 was tested in primary human airway epithelial (HAE) cultures (FIG. 1-m-FIG. 1-o). Here, Mito-MES treatment also significantly reduced the percent of SARS-CoV-2 infected human lung epithelial cells (FIG. 1-m) by a similar magnitude as observed with Mito-MES in Calu3 cells (FIG. 1-k). Flow cytometry and the marker FOXJ1 (forkhead box J1) was used to characterize the impact of Mito-MES on SARS-CoV-2 infected FOXJ1⁺ ciliated epithelial cells from ALI HAE cultures. Mito-MES treatment also significantly reduced the percent of SARS-CoV-2 infected human FOXJ1⁺ ciliated epithelial cells (FIG. 1-n). Mito-MES treatment also significantly reduced the percent of SARS-CoV-2 infected human non-cancerous HEK293T epithelial cells stably expressing ACE2 (HEK293T-hACE2) (FIG. 1-o).

These results show that Mito-MES exhibits antiviral activity against SARS-CoV-2 in cells that is more potent in interferon competent human epithelial cells. Therefore, one or more mitochondrial targeted antioxidants may be used as an antiviral against coronaviruses such as SARS-CoV-2.

Mito-MES Prevents, Inhibits, and/or Reduces Apoptosis Caused by SARS-CoV-2

The impact of Mito-MES on cellular apoptosis associated with SARS-CoV-2 infection was assessed. Immunofluorescence staining showed that SARS-CoV-2-induced increase in cleavage of caspase 3, a key regulator of cellular apoptosis, was reduced by Mito-MES treatment in both Vero (FIG. 2-e) and Calu3 cells (FIG. 2-f). Flow cytometry experiments confirmed that Mito-MES inhibits or reduces SARS-CoV-2-induced increase in both the percent of infected cells that were positive for cleaved caspase 3 (FIG. 1-p, FIG. 1-r) and the median fluorescence intensity (MFI) of cleaved caspase 3 per cell (FIG. 1-q, FIG. 1-s) in both Vero (FIG. 1-p, FIG. 1-q) and Calu3 cells (FIG. 1-r, FIG. 1-s).

These results show that Mito-MES not only has antiviral activity against SARS-CoV-2 but also prevents, inhibits, and/or reduces virus-induced cytotoxicity and apoptosis of the infected epithelial cells. Therefore, one or more mitochondrial targeted antioxidants may be used to prevent, inhibit, and/or reduce cytotoxicity and apoptosis caused by coronaviruses such as SARS-CoV-2.

These results also indicate that Mito-MES may be used to effectively prevent, inhibit, reduce, and/or treat to injury to lung cells resulting from infection by SARS-CoV-2. Therefore, one or more mitochondrial targeted antioxidants may be used to prevent, inhibit, reduce, and/or treat injury to lung cells resulting from infection by coronaviruses such as SARS-CoV-2.

### Antiviral Activity of Mito-MES is Partially Mediated Through its Antioxidant Activity

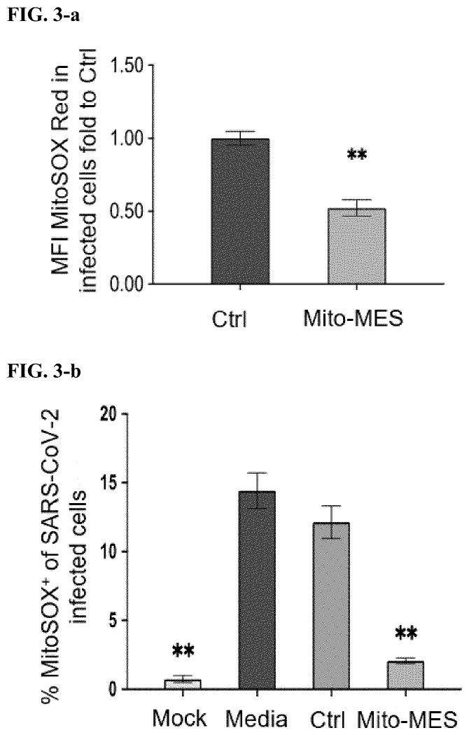

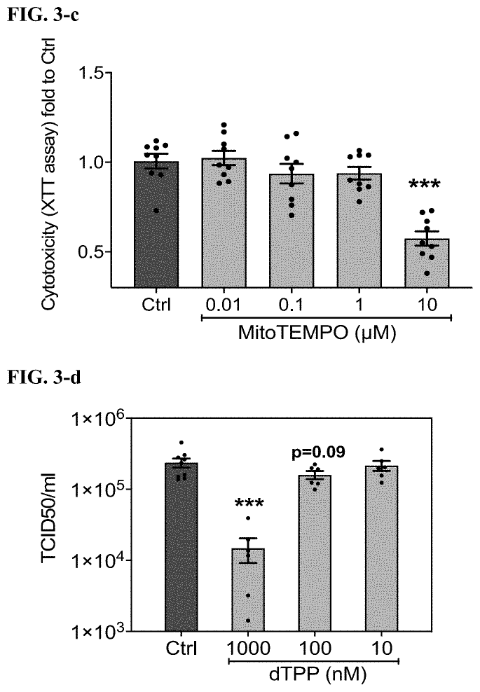

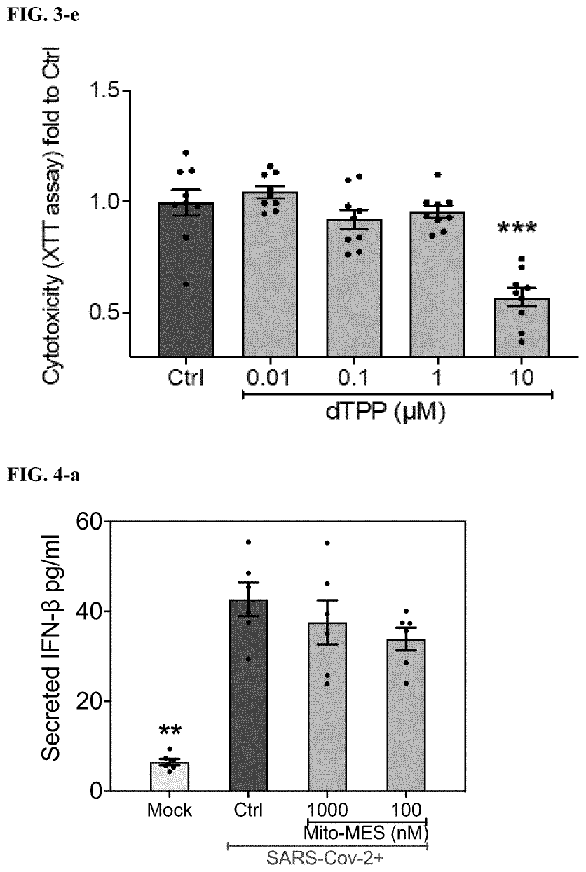

The mitochondrial pathways that may mediate the antiviral activity of Mito-MES in SARS-CoV-2 infected epithelial cells was assessed. In SARS-CoV-2 infected Calu3 cells, Mito-MES reduced mito-ROS generation as measured by reduced fluorescence (MFI and percent of cells positive for the fluorochrome) of MitoSOX Red, a mitochondrial-targeted fluorescent dye that measures (FIG. 3-a, FIG. 3-b).

To determine whether the antiviral activity of Mito-MES depends on its mitochondrial antioxidant activity, Calu3 and Vero cells infected with a reporter SARS-CoV-2 fluorescent virus were treated with an independent mitochondrial antioxidant, Mito-TEMPO, which is a SOD2 mimetic that is a known scavenger of mito-ROS and has both superoxide and alkyl radical scavenging properties. The cancerous Calu3 uninfected cells exhibited higher cellular ROS content than the non-cancerous Vero uninfected cells. Both Mito-TEMPO and Mito-MES exhibited potent and similar antiviral activity against SARS-CoV-2 in Calu3 cells at concentrations as low as 25 nM. As with Calu3 cells, both Mito-MES and Mito-TEMPO had similar antiviral activity against SARS-CoV-2 in Vero cells at concentrations as low as 25 nM. There was no observed cytotoxicity associated with Mito-TEMPO, as determined by XTT cell cytotoxicity assay, in similarly treated uninfected cultures across the dose range (10-1000 nM) (FIG. 3-c).

The impact of SARS-CoV-2 infection and Mito-MES treatment on cellular bioenergetics in Calu3 cells was assessed. SARS-CoV-2 infection reduces cellular oxygen consumption in infected cells. While 50-1000 nM Mito-MES reduced mitochondrial respirometry and increased ROS at a concentration of 1000 nM, Mito-MES exhibits antiviral activity against SARS-CoV-2 in Calu3 cells, thereby indicating its antiviral activity is not mediated through mito-ROS and alterations in cellular bioenergetics.

To determine whether Mito-MES exhibits antiviral activity against SARS-CoV-2 through its TPP+ moiety, regardless of its antioxidant activity, Calu3 cells infected with SARS-CoV-2 were treated with dTPP. Antiviral activity was observed with dTPP at a concentration of 1000 nM (FIG. 3-d), which was not cytotoxic (FIG. 3-e). Therefore, in some embodiments, dTPP without Mito-MES is administered to a subject to treat, inhibit, and/or reduce SARS-CoV-2 infection in the subject.

These results indicate that the antiviral activity of Mito-MES against SARS-CoV-2 is at least partially mediated by its antioxidant activity.

### Mito-MES Does Not Alter Host Cellular Factors Used by SARS-CoV-2 for Entry and Replication in Cells

To gain insight into the impact of Mito-MES on host cellular factors used by SARS-CoV-2 for entry in lung epithelial cells, flow cytometry was used to characterize the protein levels of ACE2, CD147, NRP1 and TMPRSS2 in Calu3 cells in response to SARS-CoV-2 infection and Mito-MES treatment. Neither SARS-CoV-2 infection nor Mito-MES significantly modulates protein levels of ACE2, TMPRSS2, NRP1 and CD147 in Calu3 cells compared to uninfected cells (data not shown). These results indicate that the antiviral activity of Mito-MES is not mediated through the pathways involving these proteins.

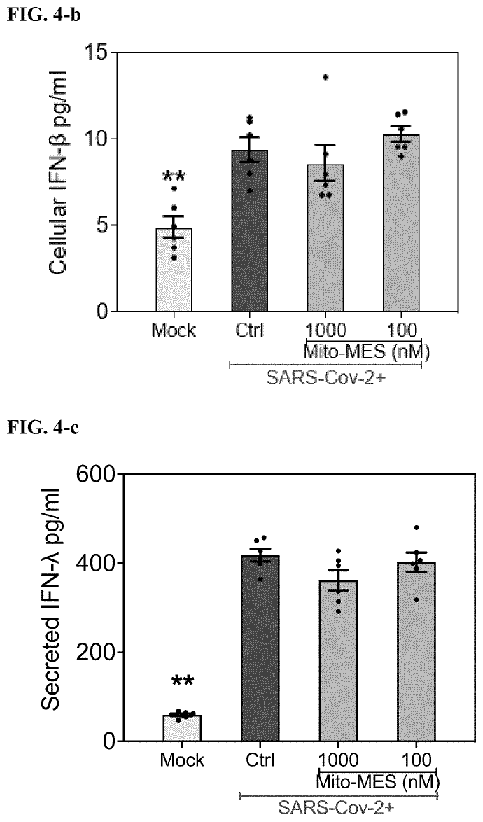

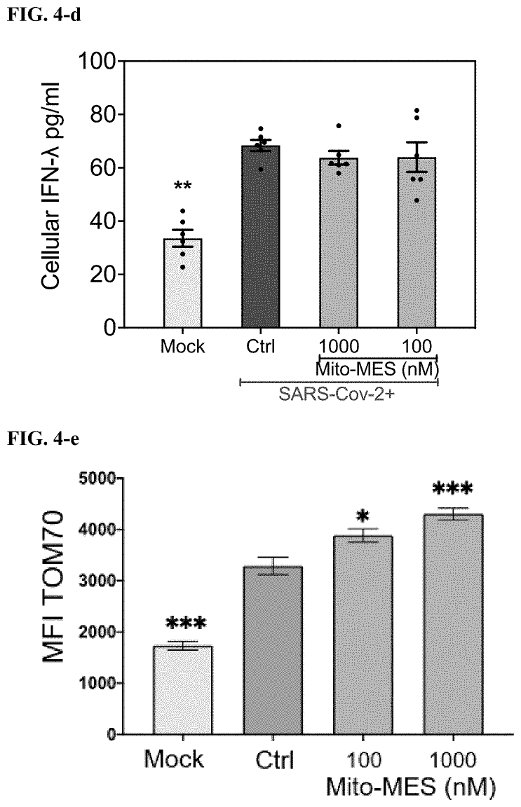

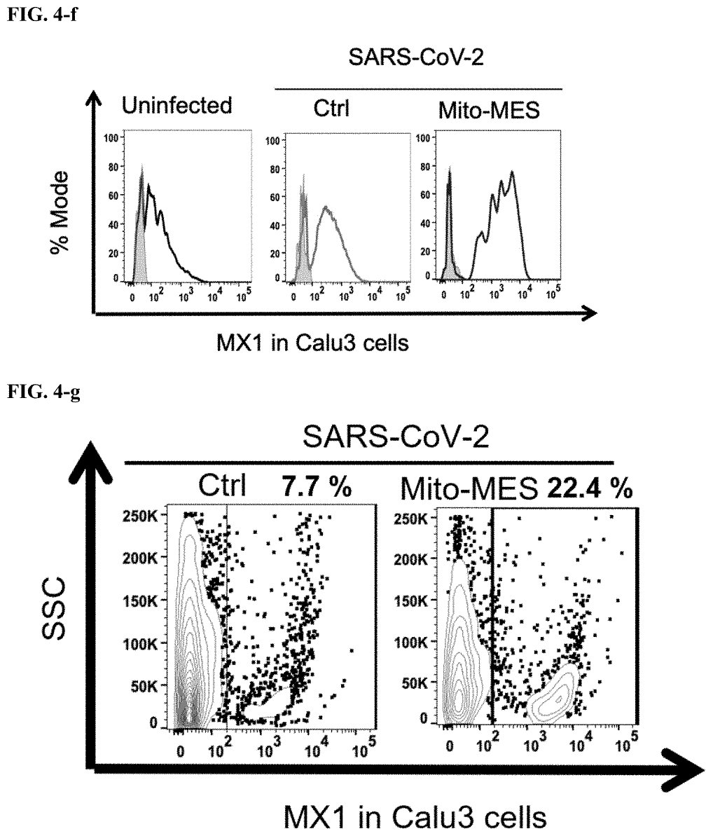

To determine whether the antiviral activity of Mito-MES is mediated through increased interferon host cellular responses, ELISA was used to measure secreted interferons (IFN-α, IFN-β, IFN-λ) in cell culture supernatants of Calu3 cells infected with SARS-CoV-2 and treated with Mito-MES. IFN-α was not detected in cell culture supernatants from Calu3 cells (data not shown). At 48 hpi SARS-CoV-2 infection increased the secretion of IFN-β and IFN-λ in infected Calu3 cells (FIG. 4-a, FIG. 4-b), while Mito-MES did not (FIG. 4-a, FIG. 4-b). These results were independently confirmed using ELISA to detect total cellular IFN-β and IFN-λ protein levels in Calu3 cells (FIG. 4-c, FIG. 4-d).

The impact of Mito-MES on mitochondrial pathways that regulate interferon host cellular responses was determined. The mitochondrial antiviral mechanism involves the activation of retinoic acid-inducible gene I (RIG-I)-like receptors (RLR) and activation of its target mitochondrial antiviral (MAV) at the mitochondrial outer membrane (MOM) to induce type I interferons (IFNs). Using flow cytometry, it was found that SARS-CoV-2 and Mito-MES did not modulate MAVS protein levels in total (infected and uninfected) and in SARS-CoV-2 infected Calu3 cells (data not shown).

The protein levels of mitochondrial protein translocase of the outer membrane 70 (TOM70) in SARS-CoV-2 infected and uninfected Calu3 cells treated with or without Mito-MES was determined. Using flow cytometry, it was found that SARS-CoV-2 and Mito-MES increased TOM70 protein levels in total (infected and uninfected) and in SARS-CoV-2 infected Calu3 cells (data not shown). Immunofluorescence experiments demonstrated that TOM70 protein levels were increased in SARS-CoV-2 infected Calu3 cells over 24-h infection compared to uninfected Calu3 cells and there was a dose-dependent increase in TOM70 protein levels in Mito-MES treated SARS-CoV-2 compared to vehicle treated infected Calu3 cells (FIG. 4-e). Notably, TOM70 fluorescence intensity (FI) was not associated with Spike S FI in SARS-CoV-2 infected Calu3 cells, but there was a strong positive association between TOM70 FI and Spike S FI in Mito-MES treated SARS-CoV-2 infected Calu3 cells (data not shown).

The protein levels of the ER adaptor called stimulator of interferon genes (STING) in SARS-CoV-2 infected Calu3 cells treated with or without Mito-MES was determined. Flow cytometry experiments demonstrated that STING protein levels increased in SARS-CoV-2 infected Calu3 cells and Mito-MES did not impact STING in SARS-CoV-2 infected Calu3 cells (data not shown).

The impact of Mito-MES on protein levels of MX-1 in infected epithelial cells was determined. Flow cytometry demonstrated that Mito-MES increased MX1 protein levels in Mito-MES treated compared to vehicle treated infected Calu3 cells (FIG. 4-f, FIG. 4-g).

Overall, these results indicate that Mito-MES does not modulate IFN-β, IFN-λ, MAVS and STING protein levels in cells infected by SARS-CoV-2, while Mito-MES induces certain mediators of type I interferon responses (TOM70 and MX1) and has better antiviral activity in interferon competent human epithelial cells. Thus, the antiviral activity of Mito-MES is not mediated through consistent upregulation of interferon cellular responses that lead to secretion of increased IFN-β and IFN-λ in cells infected with SARS-CoV-2.

### Antiviral Activity of Mito-MES is Mediated Through the Nrf2 Pathway

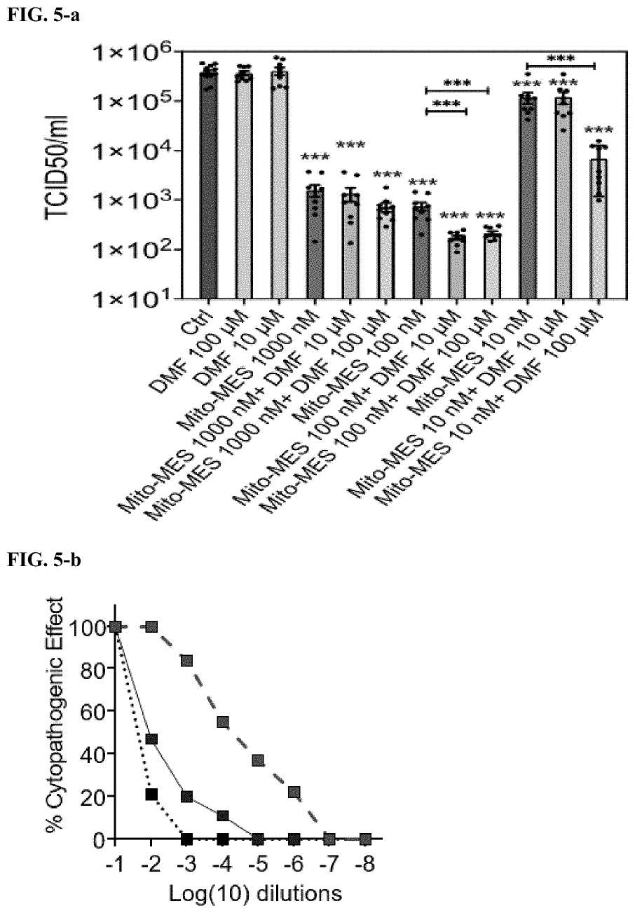

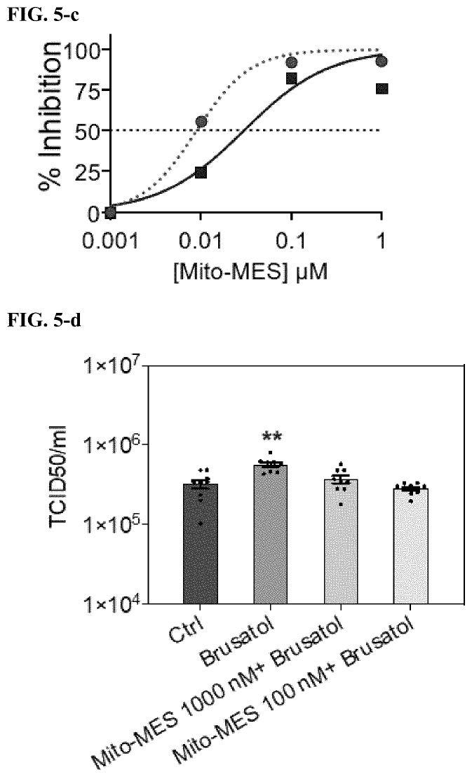

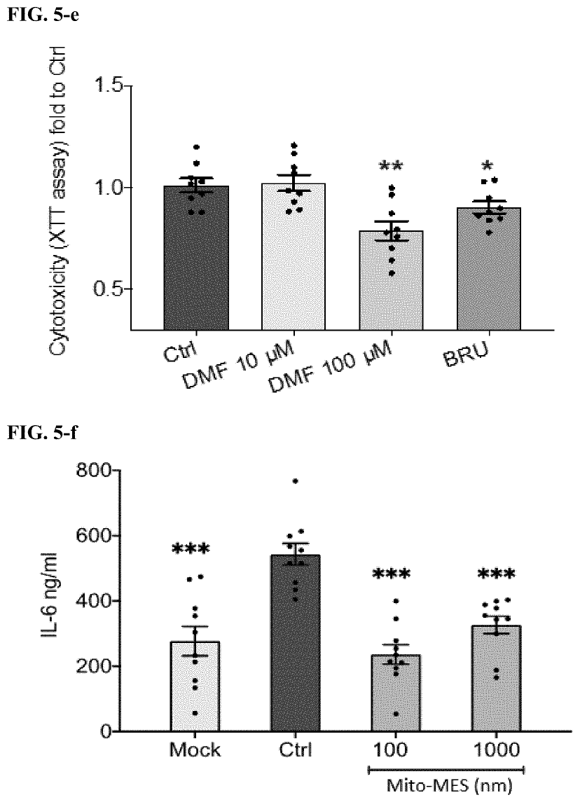

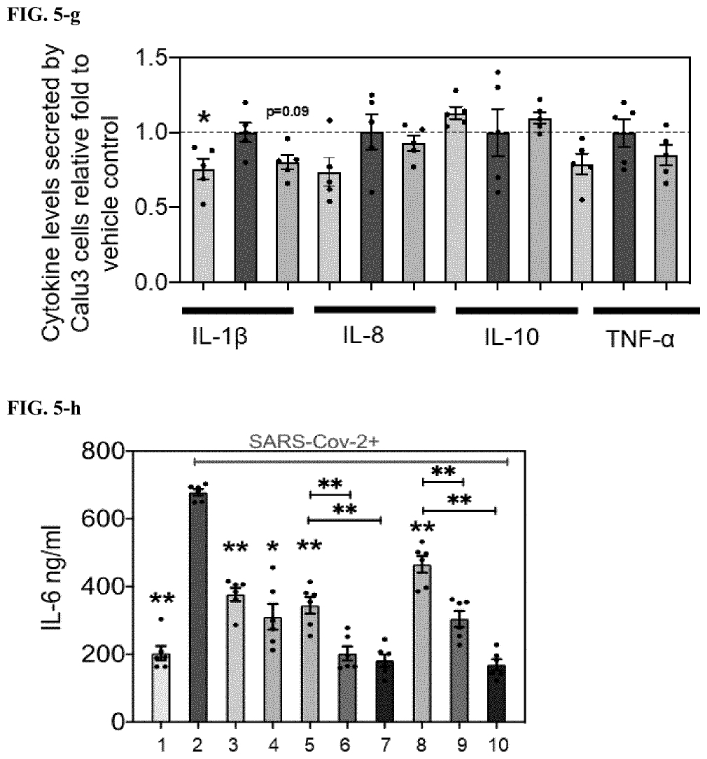

To determine whether the antiviral activity of Mito-MES against SARS-CoV-2 in lung epithelial cells is mediated through the Nrf2 antioxidant interferon-independent antiviral pathway, Calu3 cells were pretreated with the Nrf2 agonist, dimethyl fumarate (DMF) (which exhibits a cytoprotective role at lower concentrations, e.g., <25 μmol/L, through activation of the NRF2 antioxidant pathway). Pretreatment with 10 μM DMF for 24 h resulted in reduction in subsequent release of progeny SARS-CoV-2 virus particles to the cell supernatant from infected cells as measured by TCID50 assay (FIG. 5-a). Compared to pretreatment with 10 μM DMF alone, pretreatment with 10 μM DMF and 10-1000 nM Mito-MES for 24 h resulted in an additive reduction in subsequent release of progeny SARS-CoV-2 virus particles to the cell supernatant from infected cells (FIG. 5-a-FIG. 5-c). Notably, the antiviral activity of Mito-MES against SARS-CoV-2 was observed in concentrations as low as 10 nM when combined with DMF (FIG. 5-a). The combination of Mito-MES+DMF also reduced the IC50 of Mito-MES by about 3-fold (FIG. 5-c).

To further confirm that the antiviral effect of Mito-MES against SARS-CoV-2 is mediated through the Nrf2 pathway, the Calu3 cells were treated with the NRF2 small-molecule inhibitor brusatol. Pretreatment with 0.25 μM brusatol significantly increased subsequent release of progeny SARS-CoV-2 virus particles to the cell supernatant from infected cells (FIG. 5-d). Pretreatment with 0.25 μM brusatol for 24 h inhibited the antiviral effect of both DMF alone and Mito-MES (FIG. 5-d). Both 0.25 μM brusatol and 100 μM DMF, but not 10 μM DMF, were cytotoxic, as determined by XTT cell cytotoxicity assays (FIG. 5-e). These results were confirmed by flow cytometry.

The combination of 10 nM Mito-MES+10 μM DMF also significantly reduced the percent of infected Calu3 cells that were positive for the N protein of SARS-CoV-2, compared to Mito-MES treatment alone or DMF treatment alone (data not shown). Pretreatment with 0.25 μM brusatol directly enhanced SARS-CoV-2 infection and attenuated the reduction in the percent of infected Calu3 cells induced by Mito-MES and DMF induced treatments (data not shown).

Collectively, these results indicate that the antiviral activity of Mito-MES is mediated through the Nrf2 pathway.

### Impact of Mito-MES on Key Proteins of the Nrf2 Pathway

To further characterize the impact of SARS-CoV-2 infection and Mito-MES on the protein levels of the Nrf2 pathway, flow cytometry was used to determine the protein levels of NrF2 and its endogenous inhibitor, Keap1 in Calu3 cells infected with SARS-CoV-2 and pretreated with Mito-MES, DMF, and Brusatol vs vehicle control (data not shown). SARS-CoV-2 infection increased protein levels of Nrf2 and Keap (data not shown). Pretreatment with Mito-MES and DMF did not impact protein levels of Nrf2 but reduced protein levels of Keap in infected and uninfected Calu3 cells (data not shown). Pretreatment with Brusatol reduced protein levels of Nrf2 and increased protein levels of Keap in infected and uninfected Calu3 cells (data not shown).

The protein levels of HO-1 in Calu3 cells infected with SARS-CoV-2 and pretreated with Mito-MES, DMF, and Brusatol vs vehicle control was determined using flow cytometry. SARS-CoV-2 infection downregulated protein levels of HO-1 in Calu3 cells compared to uninfected cells (data not shown). Pretreatment with 10 μM DMF (but not with 100 μM DMF, 100-1000 nM Mito-MES, or the combination of Mito-MES with DMF) increased HO-1 protein levels in Calu3 cells (data not shown). Pretreatment with 0.25 μM Brusatol increased the HO-1 protein levels in SARS-CoV-2 infected cells and did not have an impact on the effect of Mito-MES on HO-1 protein levels (data not shown).

Overall, these results indicate that the antiviral effect of Mito-MES through the Nf2 pathway may not be reflected by the total cellular protein levels of Nrf2 and Keap and is not mediated by the HO-1 protein.

### Mito-MES Attenuates Release of Proinflammatory Cytokines Caused by Infection

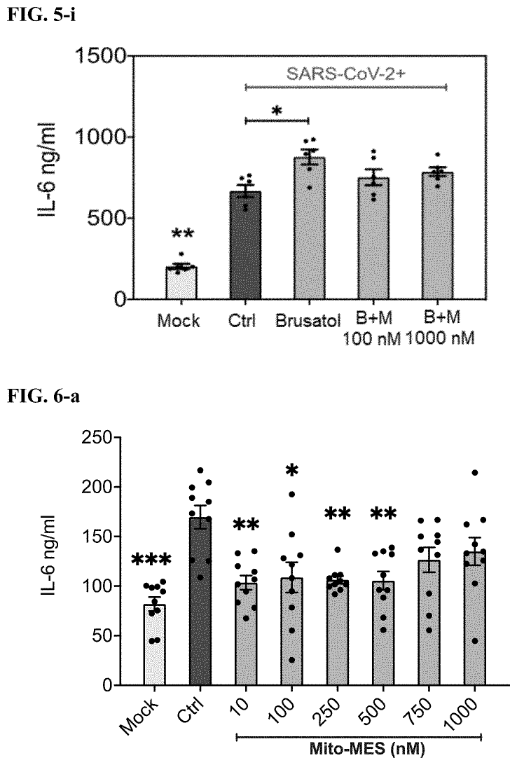

The impact of Mito-MES on inflammatory cellular responses associated with SARS-CoV-2 infection was assessed. Using ELISA, it was found that SARS-CoV-2 infection increased release of IL-6 in cell culture supernatants of both SARS-CoV-2 infected Calu3 (FIG. 5-f) and Vero-E6 (FIG. 6-a) cells. Using Luminex immunoassay, it was also found that SARS-CoV-2 infection increased secretion of IL-1β but did not impact the secretion of IL-8, IL-10, and TNF-α in cell culture supernatants of SARS-CoV-2 infected Calu3 (FIG. 5-g). Mito-MES attenuated the increase in secretion of IL-6 resulting from SARS-CoV-2 infection in Calu3 (FIG. 5-h) and Vero-E6 (FIG. 6-a) cells and tended (p=0.09) to reduce the secretion of IL-10 in cell culture supernatants of Calu3 cells infected with SARS-CoV-2 (FIG. 5-g).

Therefore, one or more mitochondrial targeted antioxidants may be used to prevent, inhibit, reduce, and/or treat inflammation resulting from infection by coronaviruses such as SARS-CoV-2.

To determine whether the anti-inflammatory activity of Mito-MES is also mediated through the Nrf2 pathway, Calu3 cells were pretreated with the Nrf2 agonist DMF and then the secretion of IL-6 by cells infected by SARS-CoV-2 was determined at 48 hpi. Pretreatment with 10 μM DMF for 24 h resulted in a reduction in subsequent release of IL-6 from infected cells as measured by ELISA (FIG. 5-h). Compared to treatment with 100-1000 nM Mito-MES alone, treatment with 10-100 μM DMF+100-1000 nM Mito-MES for 24 h further reduce the subsequent release of IL-6 from infected cells (FIG. 5-h).

To further confirm that the antiviral effect of Mito-MES against SARS-CoV-2 is mediated through the Nrf2 pathway, the Calu3 cells were treated with the NRF2 small-molecule inhibitor brusatol. Pretreatment with 0.25 μM brusatol significantly increased subsequent release of IL-6 to the cell supernatant from infected cells (FIG. 5-i) and inhibited the anti-inflammatory effect of Mito-MES (FIG. 5-i).

Overall, these results indicate that Mito-MES exhibits anti-inflammatory activity against the inflammatory response resulting from infection by SARS-CoV-2 via the Nrf2 pathway.

### Mito-MES Exhibits In Vivo Activity Against SARS-CoV-2

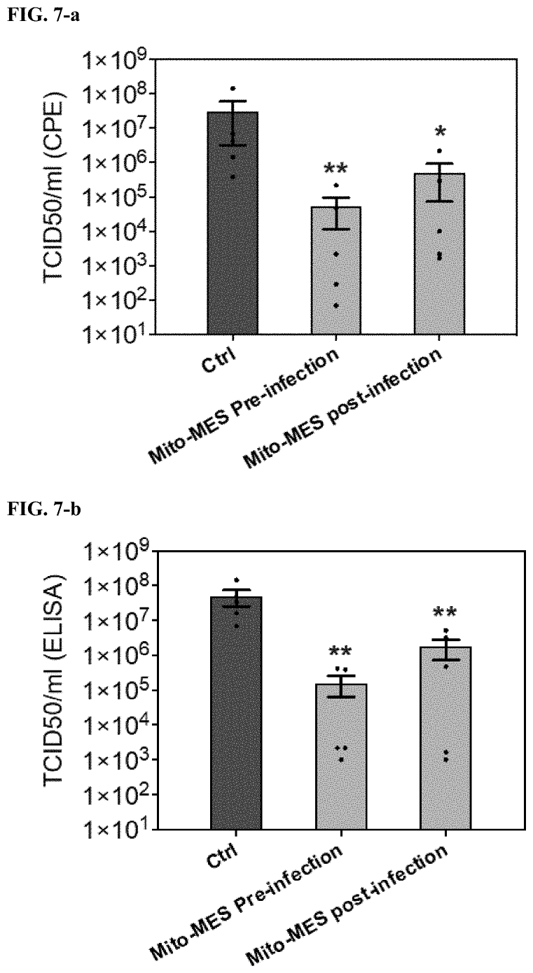

The in vivo antiviral activity of Mito-MES was evaluated in mice. It was found that Mito-MES inhibits or reduces replication of SARS-CoV-2 in vivo in subjects infected with SARS-CoV-2. Specifically, K18-hACE2 transgenic mice, which express human ACE2, were infected with SARS-CoV-2. Compared to controls (vehicle), viral titers were lower in subjects treated with Mito-MES (FIG. 7-a, FIG. 7-b).

These results indicate that Mito-MES may be used to effectively prevent, inhibit, reduce, and/or treat infections by SARS-CoV-2 in subjects.

Therefore, one or more mitochondrial targeted antioxidants may be used to prevent, inhibit, reduce, and/or treat infections by coronaviruses such as SARS-CoV-2 in subjects.

### Summary

SARS-CoV-2 infection induces production of mitochondrial reactive oxygen species (mito-ROS) and mitochondrial dysfunction. SARS-CoV-2 infection reduces the antiviral host cellular response of interferon-competent cells. Collectively, these cellular alterations drive viral replication of SARS-CoV-2 in human cells. Mitochondrial targeted antioxidants attenuate the production of mito-ROS while upregulating the antioxidant Nrf2 pathway. Mito-MES exhibits potent antiviral activity against SARS-CoV-2 in interferon competent human cells and comparatively less antiviral activity in interferon deficient cells. Mito-MES upregulates the antiviral dynamin family member M×1 that regulates mitochondrial function and antiviral cellular responses. Mito-MES upregulates the protein TOM70, that mediates the cross-talk between endoplasmic reticulum (ER) and mitochondria and induces interferon antiviral pathway responses. Mito-MES also has a hydrophobic TPP+ moiety that integrates into cellular membranes and independently exhibits antiviral activity against SARS-CoV-2. Mito-MES reduces mito-ROS and upregulates the anti-inflammatory Nrf2 pathway and also reduces the secretion of inflammatory cytokines, such as IL-6, resulting from infection by SARS-CoV-2. Each of these mechanisms of action work to prevent, inhibit, and/or reduce infection by and replication of SARS-CoV-2 in cells and subjects.

### II. Mito-MES as a Prophylactic Against SARS-CoV-2 Infection

Two parents who were repeatedly exposed to SARS-CoV-2 to their two infected children in their household without contact isolation remained asymptomatic and without virological and serological confirmation of infection despite multiple (18 in total) diagnostic tests over a period of 6 weeks after early (within one day after possible exposure) initiation of post-exposure prophylaxis with Mito-MES (in the form of MitoQ™, which was purchased over-the-counter).

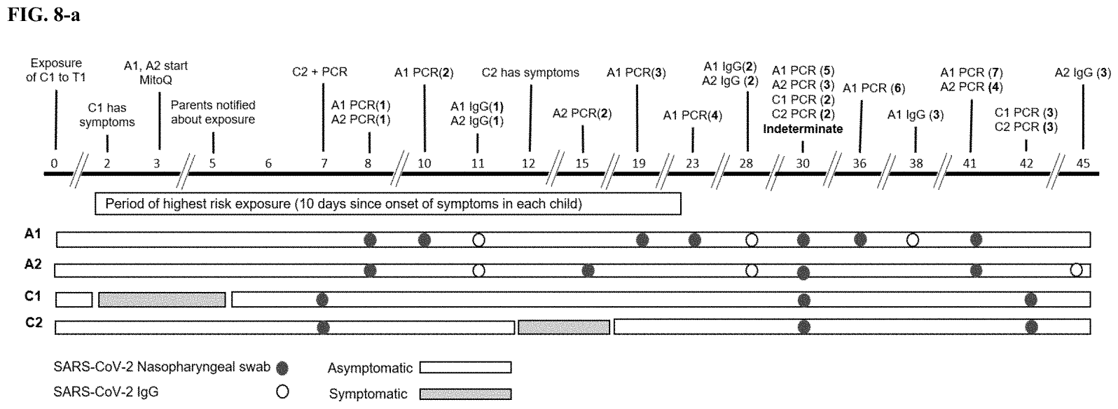

Two parents (father 40 years, A1, and mother 39 years, A2) residing in Los Angeles, Calif. were notified on Dec. 5, 2020 (defined as day 5 (D5); FIG. 8-a, C), that the schoolteacher (T1) of their 5-year-old son (C1) tested positive for SARS-CoV-2 and developed symptoms of Covid-19. C1 had 6-h exposure to T1 on November 30 (with masks) (D0). The family had no other exposure to any other person between November 20-30 (D-10 to D0) given Thanksgiving Day on November 26 (D-4). Several children and parents from the same school class were infected with Covid-19 within the first 2 weeks of December. C1 developed fever (100.4° F.; 38° C.) and fatigue on D3-D4 and remained asymptomatic since then. The second child (female 3 years, C2) was asymptomatic until D12 when she developed fever (100.4° F.; 38° C.), fatigue and coryza on D12-D14. A1 and A2 started postexposure (D3 was the first possible high grade close exposure to C1) prophylaxis with 20 mg/day oral Mito-MES after overnight fasting on D4. Physical distancing precautions were not feasible in the household. Both C1 and C2 had particularly close contact with both parents (including direct inoculation of infected saliva to mucosa of parents), throughout the period both children were unwell. Both children recovered fully without requiring medical care. Both parents remained completely asymptomatic over a period of 6 weeks with unremarkable laboratory tests on 6 different timepoints that did not demonstrate any leukocytosis, lymphopenia or evidence of inflammation including normal CRP, ferritin and IL-6 plasma levels, consistent with absence of symptoms.

C2 was SARS-CoV-2 PCR positive on nasopharyngeal (NP) swab (diagnostic sensitivity >98%) taken on D6 and D26. A1, A2 and C1 had eighteen (in total) negative NP SARS-CoV-2 PCR over a period of 5 weeks (last NP PCR was on D35) (FIG. 8-a). Each parent had undetectable levels of plasma IgG to SARS-CoV-2 S1 protein (diagnostic sensitivity >95%) obtained in three independent timepoints over a period of 5 weeks (last SARS-CoV-2 IgG serology was on D35) (FIG. 8-a). The negative predictive value of the used NP SARS-CoV-2 PCR (98%) that did not increase on further repeated testing, provides strong evidence that repeated testing for SARS-CoV-2 truly confirmed that both parents remained negative for SARS-CoV-2.

Baseline serological testing performed in both parents in less than one week since first possible exposure and multiple independent serial serological tests in both parents significantly reduced the possibility that prior protective immunity prevented the establishment of SARS-CoV-2 infection in both parents. Overall, the well-defined epidemiologic history with an exposure of C1 to T1 with confirmed Covid-19, a Covid-19 outbreak at the school of C1 and C2 around a major holiday, two positive NP PCR tests of C2 and the timing (3-7 days) of development of symptoms in both children around a well-defined exposure, confirmed that both children had SARS-CoV-2 infection.

These results indicate that Mito-MES may provide post-exposure prophylaxis against SARS-CoV-2 when administration is started within about 4 days post-exposure. Therefore, one or more mitochondrial targeted antioxidants may be administered to a subject as a prophylactic against infection by a coronavirus such as SARS-CoV-2. The one or more mitochondrial targeted antioxidants may be administered before or after (e.g., within about 4 days or less from) exposure to the coronavirus.

The Clinical and Epidemiological History Strongly Indicate that Mito-MES Inhibited Establishment of SARS-CoV-2 Infection in A1 and A2

The exposure of C1 to T1 (D0) was clearly defined around a major holiday (Thanksgiving Day) and was preceded by a 10-day period of social distancing where the family members were not exposed to other people. Given that the exposure to SARS-CoV-2 in this report was from T1 of C1 (not C2) and that C2 had positive NP SARS-CoV-2 PCR on D6 and developed symptoms on D12, it is established that C1 was contagious and transmitted the SARS-CoV-2 infection to C2. Thus, C1 was clearly contagious to both A1 and A2 especially given particularly high-grade close exposure (including direct inoculation of infected saliva to mucosa of both parents). The time of onset of symptoms in C1 is also consistent with the incubation time of SARS-CoV-2 and well-defined exposure of C1 to T1 on D0 since C1 developed fever and fatigue on D3, 3 days post exposure. In addition, C2 had positive NP SARS-CoV-2 PCR on D6, 3 days after C1 developed symptoms (D3). This means that C2, A1 and A2 had well defined SARS-CoV-2 exposure to C1 on D3 (when he developed symptoms). C2 developed symptoms on D12, i.e., 9 days post exposure to C1 (D3). Thus, the 21-day period of possible asymptomatic infection for parents A1 and A2 were between D3-D24.

It should be noted that for patients with mild to moderate COVID-19, replication-competent virus has not been recovered after 14 days following symptom onset. Recovery of replication-competent virus between 10 and 20 days after symptom onset has been documented in patients with severe Covid-19 and/or immunocompromised patients (like A1 parent). A large contact tracing study demonstrated that high-risk household and hospital contacts did not develop infection if their exposure to a case patient started late (6 days or more after the case patient's illness onset). Thus, the well-defined clinical and epidemiological history indicate that parents A1 and A2 were at particularly high risk for early high-grade (no isolation precautions were used) exposure to replication competent SARS-CoV-2 virus (from C1 and C2) between D3-D24.

Parent A1 is immunocompromised (CML) with high risk for contracting SARS-CoV-2 from C1, prolonged shedding of live virus for up to 2 months, and development of severe Covid-19. First, CML disease per se may increase the risk for development and progression of SARS-CoV-2 infection. The CML was in complete hematological remission at the time of the exposure with non-elevated white cell count and normal lymphocyte count on second generation tyrosine kinase inhibitor (TKI) (bosutinib). Patients with early and late chronic phase CML have low incidence rate of opportunistic and viral infections while on first generation TKIs like imatinib, mostly due to smaller effect on lymphocyte counts and function compared to other hematological malignancies. Second, the TKI bosutinib per se may increase the risk for development and progression of SARS-CoV-2 infection. Second generation TKIs like dasatinib and bosutinib have immunosuppressive effect on viral specific CD8⁺ T cells via inhibitory effect on Src, NF-kappaB and TCR signaling, substantially increasing the risk of A1 to contract viral infection. Third, use of steroids in A1 may increase the risk for development and progression of SARS-CoV-2 infection. A1 was on a prednisone taper for neuritis. On D3 he was taking prednisone 40 mg orally daily that was quickly tapered to 20 mg on D4-6. A1 was off steroids on D7. Use of corticosteroids significantly increases the risk for development and progression of viral infection. Fourth, A1 is blood type A which is associated with higher risk of SARS-CoV-2 infection. Thus, 6 negative PCRs over a period of 6 weeks in combination with three negative serological tests, prolonged high-grade exposure to two symptomatic children with Covid-19 without physical distancing precautions, and the high clinical risk of A1 to develop viral infection, essentially rule out the possibility that SARS-CoV-2 infection was established in A1.

Parent A2 is healthy on no medications or supplements and the absence of any clinical symptoms of SARS-CoV-2 infection in combination with 3 negative SARS-CoV-2 NP PCR (with NPV >98%), 3 negative serological tests (with >99% diagnostic sensitivity), normal white cell and lymphocyte count and normal inflammatory markers, in combination with prolonged high-grade exposure to two symptomatic children with Covid-19 without physical distancing precautions, drastically reduce the possibility that A2 was infected with SARS-CoV-2 infection and remained asymptomatic. Although parent A2 is blood type O which is associated with lower risk of SARS-CoV-2 infection, that would not prevent direct transmission of SARS-CoV-2 from C1 to A2. C2 is also blood type O and was infected, presumably from C1.

Thus, the clinical and epidemiological history strongly indicate that Mito-MES inhibited establishment of SARS-CoV-2 infection in both A1 and A2

### Mito-MES Inhibits SARS-CoV-2 Infection and Replication Via Several Mechanisms

The in vitro experiments herein, show that the antiviral activity of Mito-MES against SARS-CoV-2 was much lower in interferon deficient monkey epithelial vs interferon competent human Calu3 cells, thereby indicating that the antiviral activity of Mito-MES involves an IFN-I response. Increased mito-ROS may contribute to increased SARS-CoV-2 replication through multiple mechanisms. Mito-ROS trigger MEK, MNK1, and MAPK signaling pathways that propagate viral protein synthesis and SARS-Co-V replication. Mito-ROS also induce aberrant ER stress, lipid peroxides, alterations of membranes and proteins and activation of cytosolic phospholipases; collectively these changes may lead to induced viral replication. Thus, by targeting multiple cellular signaling pathways that are essential for viral replication in epithelial cells, including mito-ROS and IFN-I cellular responses, Mito-MES may target SARS-CoV-2 replication. Another mechanism for antiviral activity of Mito-MES against SARS-CoV-2 could be related to its antiviral host cellular responses. A notable improvement of antiviral CD8 functions was elicited by Mito-MES in viral infections like hepatitis, thereby indicating a central role for ROS in antiviral immune response. This antiviral mechanism of action of Mito-MES could be particularly important for parent A1 who is immunocompromised on bosutinib which has immunosuppressive effect on viral specific CD8⁺ T cells increasing the risk of A1 to contract viral infection. Mito-MES is also anti-inflammatory and attenuates major pro-inflammatory cytokines.

Importantly, both A1 and A2 parents had undetectable inflammatory markers (IL-6, ferritin, CRP), consistent with absence of development of symptomatic SARS-CoV-2 infection. Therefore, one or more mitochondrial targeted antioxidants may be used to prevent, inhibit, and/or reduce the inflammatory response resulting from infection by SARS-CoV-2 that contributes to morbidity in COVID-19.

### III. ApoA-I Mimetic Peptide 4F Attenuates In Vitro Replication of SARS-CoV-2, Associated Apoptosis, Oxidative Stress, and Inflammation in Cells

The experiments herein show that the ApoA-I mimetic peptide 4F has antiviral activity against SARS-CoV-2 in Calu3 and Vero-E6 cells. In SARS-CoV-2 infected Calu3 cells, 4F upregulated inducers of the interferon pathway such as MX-1 and Heme oxygenase 1 (HO-1) and downregulated mitochondrial reactive oxygen species (mito-ROS) and CD147, a host protein that mediates viral entry. 4F also reduced associated cellular apoptosis and secretion of IL-6 in both SARS-CoV-2 infected Vero-E6 and Calu3 cells. Thus, 4F attenuates in vitro SARS-CoV-2 replication, associated apoptosis in epithelial cells and secretion of IL-6, a major cytokine related to COVID-19 morbidity.

### 4F Exhibits Antiviral Activity Against SARS-CoV-2

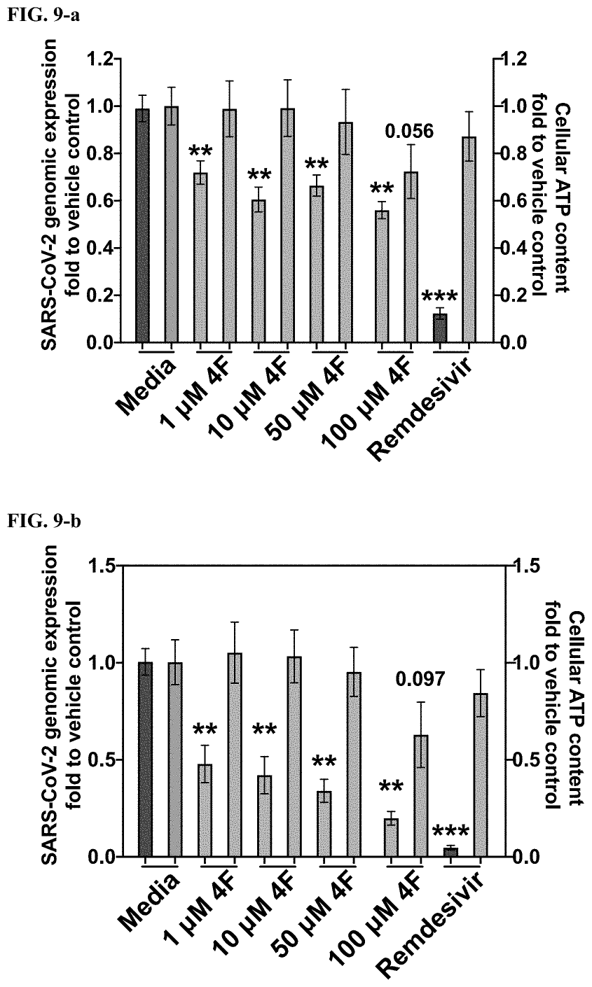

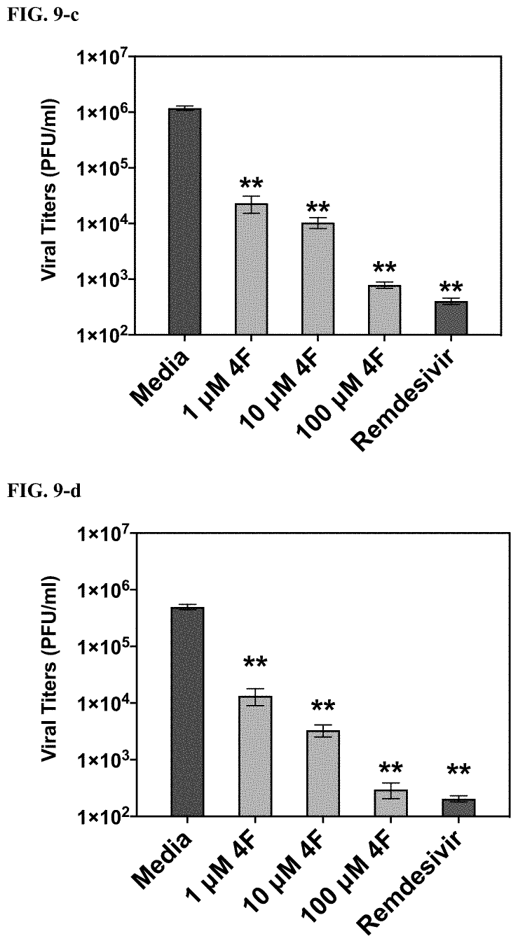

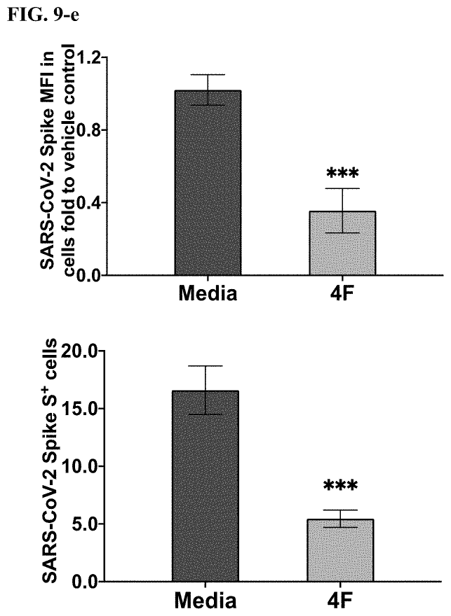

Vero E6 and Calu3 cells were pretreated for 24 h (h) with 4F (1-100 μM) and were subsequently infected with a clinical isolate of SARS-CoV-2 (multiplicity of infection (MOI) of 0.1) for 48 h in 4F- or remdesivir-containing medium. Remdesivir was included as a positive control for antiviral effect. Using qPCR that assesses viral genomic RNA in cell lysates, it was found that 4F inhibited SARS-CoV-2 in both Vero E6 (FIG. 9-a) and Calu3 cells (FIG. 9-b). There was no observed cytotoxicity associated with 4F in similarly treated uninfected cultures across the 1-10 μM dose range (FIG. 9-aFIG. 9-b). These results were corroborated by a reduction in viral titer, where 4F at concentrations 1-100 μM displayed similar reduction in viral titer in both Vero E6 (FIG. 9-c) and Calu3 cells (FIG. 9-d). A concentration 10 μM was chosen for all future experiments.

Using immunofluorescence, 4F was confirmed to exhibit antiviral activity in Calu3 cells (FIG. 9-e). Flow cytometry was used to characterize the cellular protein levels of the Spike S protein in infected cells. Immunofluorescence data (FIG. 9-e) at the single cell level in viable cells was used to confirm that 4F induced a more prominent reduction in the percent of infected cells that were positive for Spike S protein compared to the Spike S MFI in infected Vero E6 and Calu3 (data not shown) cells.

These results indicate that ApoA-I mimetic peptides exhibit antiviral activity against SARS-CoV-2. Therefore, ApoA-I mimetic peptides may be used to prevent, inhibit, and/or reduce infections by and replication of coronaviruses such as SARS-CoV-2.

### 4F Exhibits Antioxidant Activity Against SARS-CoV-2

The potential cellular mediators that may mediate the antiviral effect of 4F in SARS-CoV-2 infected epithelial cells was determined. It was found that 4F upregulates heme oxygenase 1 (HO-1) in epithelial cells. Specifically, Calu3 cells were uninfected (mock) or infected with SARS-CoV-2 (MOI 0.1) and were treated with media alone (vehicle), or 4F (10 μM) as in methods. Confluent cells were fixed 48 h hpi followed by processing for staining (flow cytometry) for HO-1, mito-ROS, antiviral MX1, and CD147. Flow cytometry was used in Calu3 cells to determine the percent of cells positive for each target compared to a negative cell population. Flow cytometric staining was performed at 48 hpi for HO-1, flow cytometric staining for the fluorochrome MitoSOX Red as a measure of mitochondrial reactive oxygen species (mito-ROS) content in Calu3 cells was performed at 48 hpi, flow cytometric staining was performed at 48 hpi for MX1, and flow cytometric staining for CD147 was performed at 48 hpi. Flow cytometry experiments demonstrated that HO-1 protein levels were decreased in SARS-CoV-2 infected compared to uninfected Calu3 cells and 4F increased HO-1 protein expression in 4F treated SARS-CoV-2 compared to vehicle treated infected Calu3 cells (data not shown). Consistent with the antioxidant effect of 4F, the MFI of MitoSOX red, a measure of cellular content for mitochondrial reactive oxygen species (mito-ROS) was also reduced in 4F treated compared to vehicle treated infected Calu3 cells (data not shown).

### Impact of 4F on Interferon Antiviral Responses

The impact of 4F on protein levels of MX1, a key antiviral effector in COVID-19 patients, in infected epithelial cells was determined. Flow cytometry experiments demonstrated that MX1 protein levels were decreased in SARS-CoV-2 infected compared to uninfected Calu3 cells and 4F increased MX1 protein expression in 4F-compared to vehicle treated infected Calu3 cells (data not shown). These results indicate that the antiviral activity of 4F against SARS-CoV-2 is partially mediated through the interferon pathway.

### Impact of 4F on the Viral Entry Mediator CD147

4F attenuates CD147 protein levels in SARS-CoV-2 infected cells. Specifically, flow cytometry experiments demonstrate that CD147 protein levels were increased in SARS-CoV-2 infected compared to uninfected Calu3 cells and 4F reduced CD147 protein expression in 4F treated compared to vehicle treated infected Calu3 cells (data not shown). Thus, 4F may reduce SARS-CoV-2 replication in cells by reducing the viral entry host protein CD147.

### 4F Attenuates Apoptosis Caused by SARS-CoV-2

The impact of 4F on cellular apoptosis associated with SARS-CoV-2 infection was assessed. Flow cytometry showed that 4F attenuated SARS-CoV-2-induced increase in both the percent of infected cells that were positive for cleaved caspase 3 and the MFI of cleaved caspase 3 per cell in both Vero E6 and Calu3 cells (data not shown).

These results indicate that 4F not only has antiviral activity against SARS-CoV-2 but also prevents, inhibits, and/or reduces apoptosis caused by SARS-CoV-2. Therefore, ApoA-I mimetic peptides may be used to prevent, inhibit, reduce, and/or treat injury to lung cells resulting from infection by coronaviruses such as SARS-CoV-2.

### 4F Attenuates Release of Proinflammatory Cytokines Caused by SARS-CoV-2

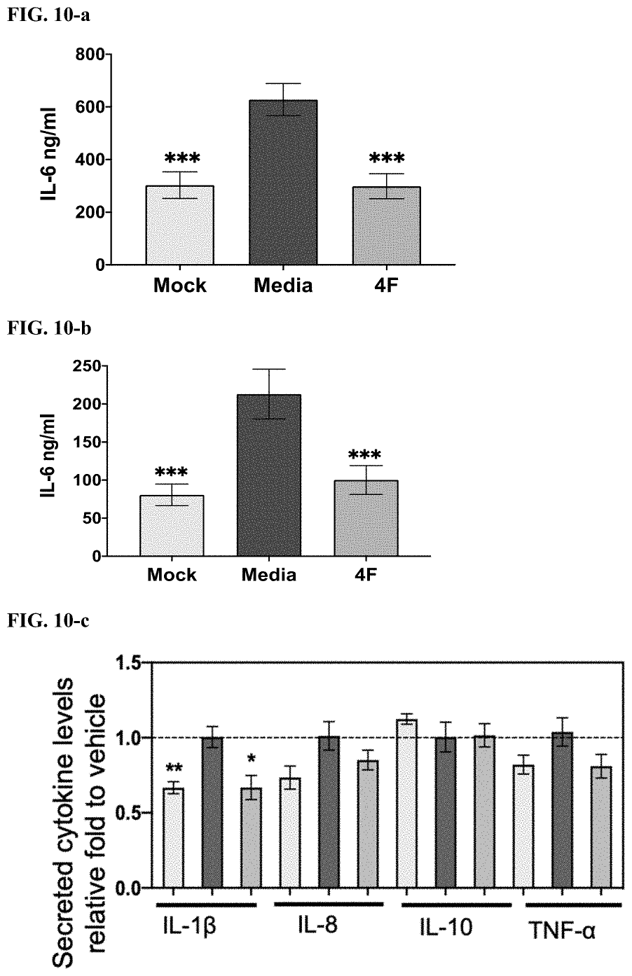

The anti-inflammatory impact of 4F on SARS-CoV-2 infected cells was assessed. Using ELISA and Luminex immunoassays, it was found that 4F attenuated SARS-CoV-2-induced increase in secretion of IL-6 by both Calu3 (FIG. 10-a) and Vero E6 (FIG. 10-b) cells and secretion of IL-1β in cell culture supernatants of SARS-CoV-2 infected Calu3 cells (FIG. 10-c). There was no impact of SARS-CoV-2 or 4F on IL-8, IL-10 and TNF-α in cell culture supernatants of SARS-CoV-2 infected Calu3 (FIG. 10-c). Thus, 4F attenuated secretion of both IL-6 and IL-1β by infected SARS-CoV-2 cells. Therefore, ApoA-I mimetic peptides may be used to prevent, inhibit, reduce, and/or treat inflammation resulting from infection by coronaviruses such as SARS-CoV-2.

### Summary

4F exhibits antiviral, antioxidant, antiapoptotic, and anti-inflammatory activity in cells that are targeted by SARS-CoV-2. 4F induces membrane related changes in cytoplasmic membrane that lead to reduced CD147, caspase 3, mito-ROS, and proinflammatory responses (secretion of IL-6). 4F also induces expression of HO-1 that further promote interferon antiviral responses (like MX-1). Each of these mechanisms result in the antiviral, antioxidant, antiapoptotic, and anti-inflammatory activity of 4F against SARS-CoV-2.

### IV. Combination Therapies

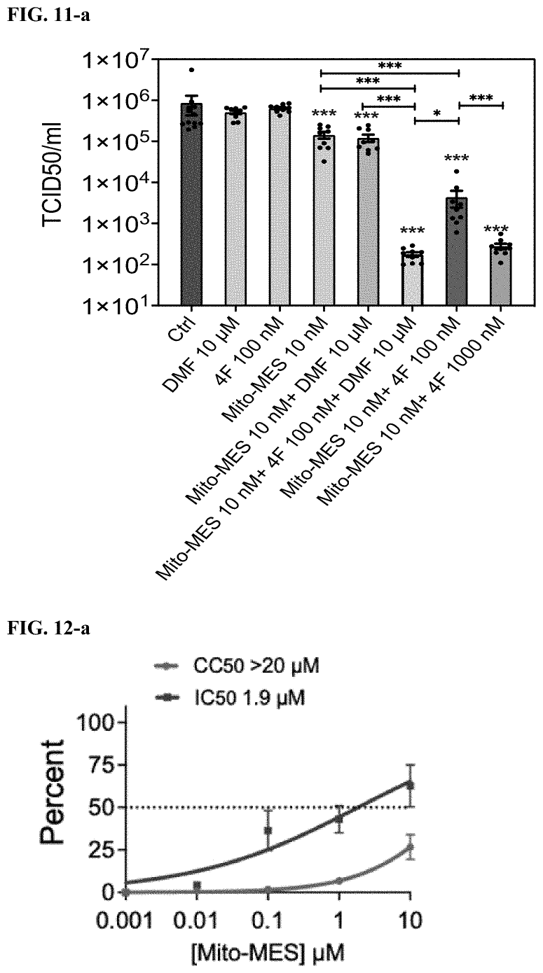

The antiviral activity of Mito-MES against SARS-CoV-2 in epithelial cells is amplified with the combination use of Mito-MES with dimethyl fumarate and/or the ApoA-I mimetic peptide 4F. The results are summarized in FIG. 11-a.

To determine whether the 3 agents have additive antiviral effects against SARS-CoV-2, the effect of the following combinations were determined:

Because (1) a concentration of 10 μM DMF alone did not produce an observable antiviral effect in human lung epithelial cells, (2) a concentration of 100 nM 4F alone did not produce an observable antiviral effect in human lung epithelial cells, (3) a concentration of 1000 nM 4F resulted in an observable antiviral effect in human lung epithelial cells, and (4) a concentration of 10 nM Mito-MES was the lowest concentration that resulted in an observable antiviral effect in human lung epithelial cells, the antiviral effect of the following combinations were assayed:

Mito-MES (10 nM)+DMF (10 μM);

Mito-MES (10 nM)+DMF (10 μM)+4F (100 nM);

Mito-MES (10 nM)+4F (100 nM); and

Mito-MES (10 nM)+4F (1000 nM).

Specifically, human lung epithelial cells (Calu3 cells) were treated with Mito-MES (10 nM, 72 hrs: 24 h before infection and 48 h during infection) and/or DMF (10 μM, 24 h before infection) and/or 4F (100 nM, 1000 nM, 72 hrs: 24 h before infection and 48 h during infection) and were infected with SARS-CoV-2 (48 h) (MOI 0.1). Viral replication by TCID50-assay at 48 hpi were determined in supernatants of Calu3 cells. It was found that:

10 nM Mito-MES reduced viral titers of infected cells compared to those treated with vehicle control (Ctrl);

10 μM DMF did not reduce viral titers of infected cells compared to those treated with vehicle control (Ctrl);

100 nM 4F did not reduce viral titers of infected cells compared to those treated with vehicle control (Ctrl);

10 nM Mito-MES+10 μM DMF reduced viral titers of infected cells compared to those treated with vehicle control (Ctrl);

10 nM Mito-MES+100 nM 4F reduced viral titers of infected cells compared to those treated with vehicle control (Ctrl), which amount of reduction was more than the total sum of the amounts of reduction provided by 10 nM Mito-MES and 100 nM 4F;

10 nM Mito-MES+1000 nM 4F reduced viral titers of infected cells compared to those treated with vehicle control (Ctrl), which amount of reduction was even more than that provided by 10 nM Mito-MES+100 nM 4F; and

10 nM Mito-MES+100 nM 4F+10 μM DMF reduced viral titers of infected cells compared to those treated with vehicle control (Ctrl), which amount of reduction was more than the total sum of the amounts of reduction provided by 10 μM DMF and 10 nM Mito-MES+1000 nM 4F.

### Summary

These results show that the combination of Mito-MES+4F results in a synergistic antiviral activity. These results also indicate that addition of DMF to Mito-MES+4F provides a further synergistic effect. That is, when DMF is included in the combination, about 1/10^{th} the amount of 4F may be used and yet achieve about the same or moderately better antiviral activity. Based on these observations and the mechanisms through which Mito-MES and 4F exert their antiviral effects against SARS-CoV-2, it is expected that other mitochondrial targeted antioxidants combined with other ApoA-I mimetic peptides will also exhibit synergistic antiviral effects against coronaviruses including SARS-CoV-2.

Therefore, in some embodiments, the combination of one or more mitochondrial targeted antioxidants and one or more ApoA-I mimetic peptides are used to prevent, inhibit, reduce, and/or treat infections by and replication of coronaviruses such as SARS-CoV-2. In some embodiments, the combination further includes an Nrf2 agonist such as DMF.

### Mito-MES Decreases SARS-CoV-2 Replication In Vitro in Independent Epithelial Cells.

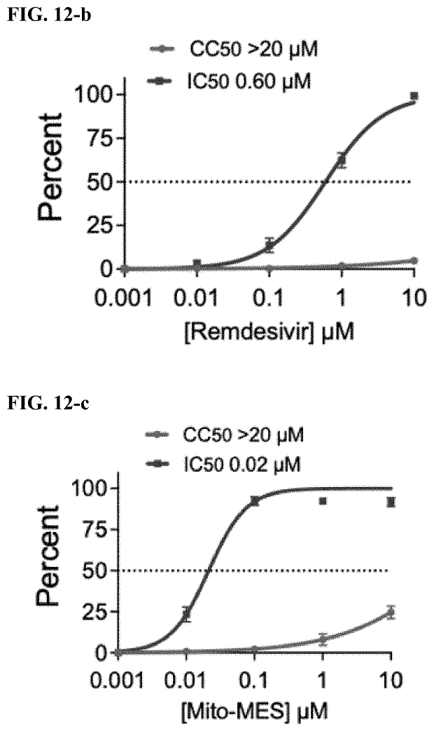

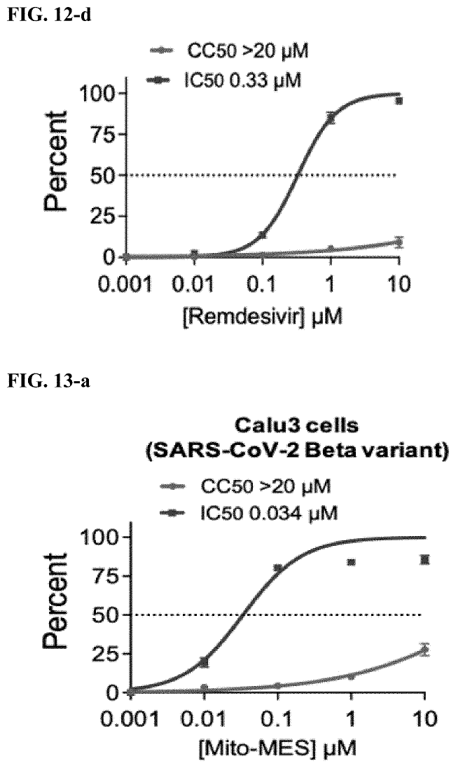

Mito-MES inhibition of SARS-CoV-2 replication was tested in Vero E6 cells. Using TCID50 assay Mito-MES was found to have a mean three orders of magnitude lower half maximal inhibitory concentration (IC50) against SARS-CoV-2 (FIG. 12-a), compared to remdesivir (RDV) (FIG. 12-b). The antiviral effect of 100-750 nM Mito-MES was confirmed by immunofluorescence (IF) detection of SARS-CoV-2 and by qPCR. Given that levels of viral proteins better reflect cellular coronavirus infection, flow cytometry was used to quantify the cellular levels of the Spike S and the Nucleocapsid N proteins of SARS-CoV-2 in infected cells. Flow cytometry confirmed that 1000 nM Mito-MES reduced the percent of cells that were positive for SARS-CoV-2 N and Spike S proteins. There was no observed cytotoxicity induced by Mito-MES across the effective dose range (10-1000 nM) in non-infected cells, as determined by XTT cell cytotoxicity assay (FIG. 12-a).

Mito-MES was tested in IFN-I responsive human lung epithelial Calu-3 (Calu3) cells. Notably, the IC50 of Mito-MES against SARS-CoV-2 as measured using TCID50 analysis of cell supernatants was 16 orders of magnitude lower (FIG. 12-c) compared to remdesivir (FIG. 12-d). The anti-SARS-CoV-2 activity of 10-1000 nM Mito-MES as assessed by immunofluorescence and qPCR was much more potent in Calu3 cells compared to Vero E6 cells in as low concentration as 10 nM. Flow cytometry further confirmed at the protein level that Mito-MES attenuated the percent of SARS-CoV-2 infected Calu3 cells. The anti-SARS-CoV-2 activity of Mito-MES was also confirmed in HEK 293T expressing ACE2. Mito-MES treatment also reduced the percent of SARS-CoV-2 infected human non-cancerous airway epithelial (HAE) cells in air-liquid interface (ALI) cultures by a mean 20 orders of magnitude. Flow cytometry showed that Mito-MES treatment significantly reduced the percent of human FOXJ1 (forkhead box J1) positive ciliated epithelial cells infected with SARS-CoV-2. In summary, these results show that, Mito-MES has potent antiviral activity against SARS-CoV-2 at the nM level in interferon competent human epithelial cells. This data is consistent with evidence indicating that SARS-CoV-2 infection triggers mito-ROS production in monocytes and pretreatment with Mito-MES inhibited SARS-CoV-2 replication in these cells. Thus, Mito-MES directly attenuates SARS-CoV-2 replication in lung epithelial cells, the main target of SARS-CoV-2.

### Mito-MES Shows Antiviral Efficacy Against SARS-CoV-2 Variants of Concerns (VOCs) and Other Coronaviruses

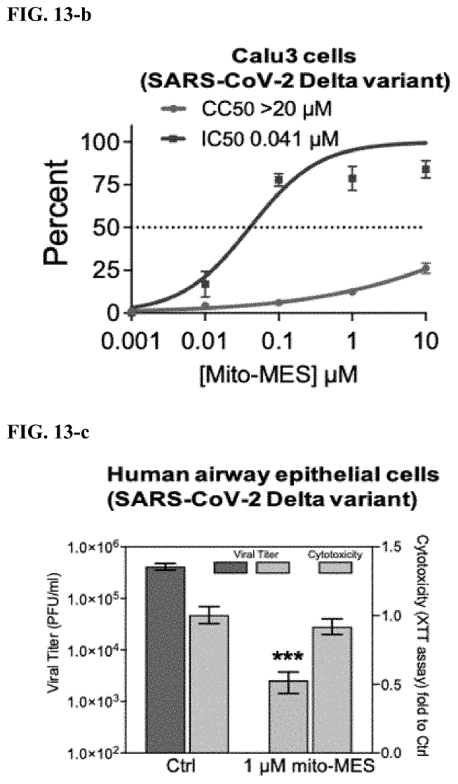

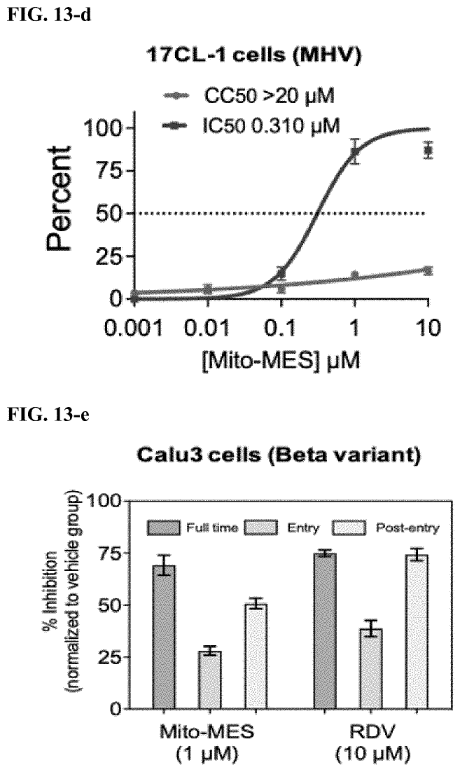

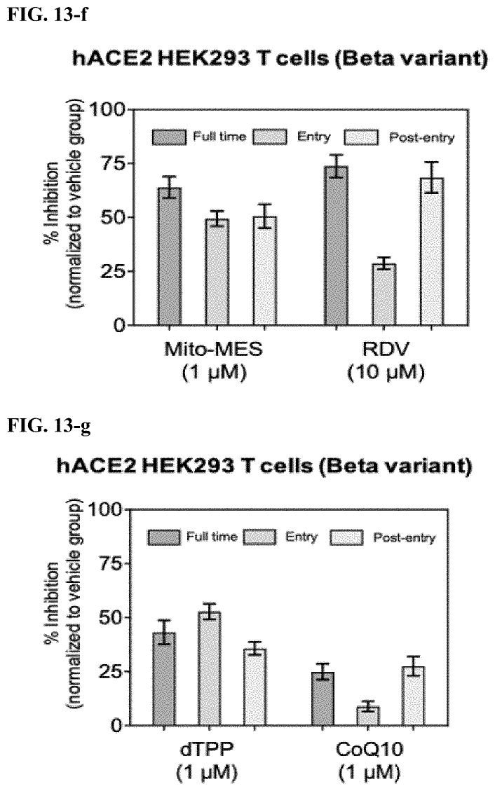

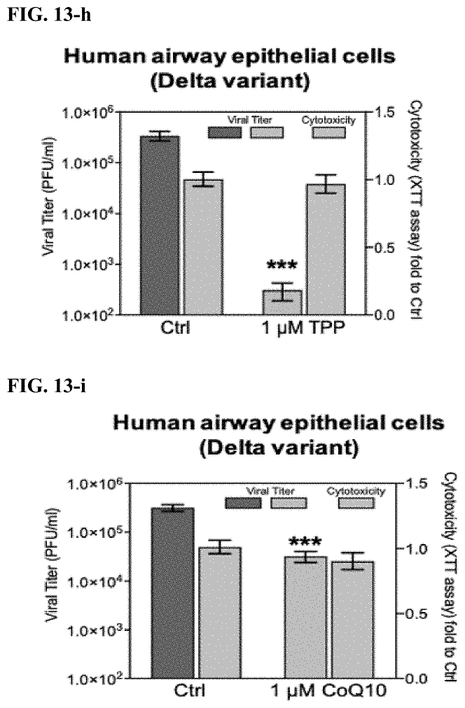

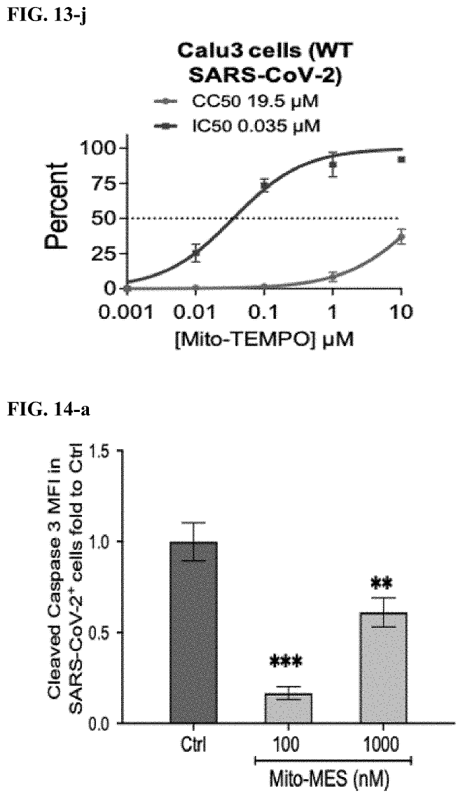

Based on initial experiments, it was hypothesized that Mito-MES has also antiviral activity against SARS-CoV-2 VOCs such as the B.1.351 (Beta) and the B.1.617.2 (Delta) variant and other coronaviruses such as the murine coronavirus hepatitis virus A-59 (MHV-A59). Mito-MES inhibited in vitro SARS-CoV-2 Beta (FIG. 13-a) and Delta variants (FIG. 13-b) in Calu3 cells with an IC50 of 0.034 μM and 0.041 μM, respectively (FIG. 13-a, FIG. 13-b). Mito-MES 1000 nM also inhibited SARS-CoV-2 Delta variants in ALI lung cultures (FIG. 13-c). Mito-MES 1000 nM had also anti-MHV-A59 activity in mouse 17C1-1 fibroblast cells (FIG. 13-d). This data suggest that Mito-MES can be a useful antiviral therapy for coronaviruses, especially in the setting of rapidly emerging SARS-CoV-2 variants.

### The Antiviral Activity of Mito-MES in Epithelial Cells is Partially Mediated Through its TPP Moiety.

To elucidate the mechanism of action through which Mito-MES inhibits SARS-CoV-2, time-of-addition studies were performed, which compared the effects of Mito-MES administered before, concurrently with viral infection with those of the Mito-MES added four hours after virus challenge, which allows time for viral entry. 1000 nM Mito-MES inhibited nucleocapsid protein expression in Calu3 cells when added before infection (effect on viral entry) but also even when added 4 hours after infection in both Calu3 (FIG. 13-e) and hACE2 HEK293T cells infected with Beta variant of SARS-CoV-2. These results suggest that Mito-MES inhibits both viral entry and cytoplasmic replication.

To gain further insight into the impact of Mito-MES on host cellular factors used by SARS-CoV-2 for entry in lung epithelial cells, flow cytometry showed that Mito-MES did not impact the levels of ACE2, TMPRSS2, CD147 and NRP1 proteins in SARS-CoV-2 infected and uninfected Calu3 cells. Viral titer assays in both hACE2 HEK293T cells infected with Beta variant (FIG. 13-g) and ALI HAE cultures infected with Delta variant (FIG. 13-h) that 1000 nM confirmed that dTPP was not cytotoxic but had potent antiviral activity against both the SARS-CoV-2 Beta stain (FIG. 13-g) as well as the Delta (B.1.617.2) SARS-CoV-2 variant (FIG. 13-h). Time-of-addition studies in hACE2 HEK293T cells showed that dTPP reduced SARS-CoV-2 entry and had less impact on SARS-CoV-2 cytoplasmic replication (FIG. 13-g). Collectively, these data showed that antiviral activity of Mito-MES in epithelial cells is partially mediated through its TPP moiety.

### Interferon Pathways as Mediators of the Antiviral Activity of Mito-MES Against SARS-CoV-2 in Human Lung Epithelial Cells.

Given data that the antiviral activity of Mito-MES was lower in interferon deficient Vero E6 cells compared to interferon competent human epithelial cells, cytosolic antiviral activity of Mito-MES may be mediated through interferon responses. Using flow cytometry, Mito-MES had no impact on mitochondrial antiviral signaling (MAVS) but increased the translocase of the outer membrane 70 (TOM70) protein levels in total (infected and uninfected) and in SARS-CoV-2 infected Calu3 cells. The Mito-MES-induced increase in TOM70 in SARS-CoV-2 infected Calu3 cells was confirmed by immunofluorescence. Mito-MES did not impact stimulator of interferon genes (STING) in SARS-CoV-2 infected Calu3 cells. Flow cytometry demonstrated that Mito-MES increased MX1 protein levels in Mito-MES treated compared to vehicle treated uninfected Calu3 cells. Secreted and cellular interferons (IFN-β, IFN-λ) were measured in Calu3 cells infected with SARS-CoV-2 and treated with Mito-MES using ELISA. At 48 hours post SARS-CoV-2 infection, Mito-MES did not impact increase in secreted and cellular IFN-β (S4G, H) and IFN-λ (S4I, J) in infected Calu3 cells. Overall, this data demonstrate that Mito-MES does not impact IFN-β, IFN-λ, MAVS and STING protein levels in SARS-CoV-2 infected Calu3 cells, while it induces certain mediators of IFN-I (TOM70 and MX1) and has better antiviral activity in interferon competent human epithelial cells.

### The Antiviral Activity of Mito-MES in Epithelial Cells is Partially Mediated Through its Antioxidant Activity.

In SARS-CoV-2 infected Calu3 cells, flow cytometry showed that Mito-MES treatment reduced fluorescence of the mitochondrial superoxide reporter MitoSOX Red. To determine whether the antiviral activity of Mito-MES depends on its mitochondrial ROS scavenging activity, Calu3 and Vero E6 cells were treated with Mito-TEMPO, a Superoxide dismutase 2 (SOD2) mimetic that has the TPP moiety as well as superoxide and alkyl radical scavenging properties. Both Mito-TEMPO and Mito-MES had potent and similar antiviral activity against SARS-CoV-2 in Calu3 cells (FIG. 13-j) and Vero E6 cells in non-cytotoxic concentrations as high as 1000 nM and as low as 25 nM (FIG. 13-j). Unlike Mito-TEMPO, only higher concentrations (1000-2000 nM) of Coenzyme Q10 (CoQ10), an established mito-ROS scavenger which does not have the TPP moiety, had antiviral activity against both the Beta (FIG. 13-g) and the Delta strain (FIG. 13-i) in hACE2 HEK293T cells (FIG. 13-j) and ALI lung cultures (FIG. 13-i).

Treatment with 1000 nM Mito-MES increased the ROS content of Calu3 cells, while preserving potent antiviral activity (data not shown). Mito-MES treatment at concentrations 50-1000 nM that displayed anti-SARS-CoV2 activity reduced mitochondrial oxygen consumption. Consequently, mitochondrial respirometry data further supported that mitochondrial scavenging of ROS or alteration in mitochondrial function are not the main mediators of the anti-SARS-CoV-2 activity of Mito-MES in Calu3 cells. Thus, the anti-SARS-CoV-2 activity of Mito-MES may be mediated through effects on cytosolic non-mitochondrial cellular targets. Since the TPP moiety is part of both Mito-MES and mito-TEMPO but not CoQ10, this data suggest that the anti-SARS activity of Mito-MES is mediated partially through both its TPP moiety and its antioxidant activity.

### The Antiviral Activity of Mito-MES Against SARS-CoV-2 in Calu3 Cells is Mediated Through the Nrf2 Antioxidant Pathway.

To determine whether the antiviral activity of Mito-MES against SARS-CoV-2 in lung epithelial cells is mediated through the Nrf2 pathway, Calu3 cells were pretreated with the Nrf2 agonist Dimethyl fumarate (DMF) that is cytoprotective at lower concentrations (<25 μM). Pretreatment of Calu3 with 10 μM DMF for 24 hours had anti-SARS-CoV-2 activity in Calu3 cells (FIG. 5-a) and increased anti-SARS-CoV-2 activity of 10-1000 nM Mito-MES as measured by TCID50 assay (FIG. 5-a). The additive anti-SARS-CoV-2 activity of Mito-MES was less when a less cytoprotective higher (100 μM) concentration of DMF was used (FIG. 5-a). The combination of Mito-MES with DMF also reduced the IC50 of Mito-MES by 3-fold (FIG. 5-c). These data were confirmed by flow cytometry. Pretreatment with Mito-MES and DMF did not impact protein levels of Nrf2 but reduced protein levels of the endogenous Nrf2 inhibitor, Keap1 in infected and uninfected Calu3 cells. Pretreatment of Calu3 cells with 10 μDMF but not with 100 μM DMF, 100-1000 nM Mito-MES or the combination of Mito-MES with DMF, increased protein levels of heme oxygenase-1 (HO-1), a key antiviral protein of the Nrf2 pathway.

Pretreatment of Calu3 for 24 hours with non-cytotoxic (FIG. 5-e) 0.25 μM dose of the NRF2 small-molecule inhibitor brusatol significantly increased SARS-CoV2 replication as assessed by TCID50 (FIG. 5-d) and flow cytometry (FIG. 13-f) and inhibited the antiviral effect of both DMF alone and Mito-MES. Viral replication by flow cytometry at 48 hpi in Calu3 cells treated with DMF and/or Mito-MES (no infection 0.01%, control 9.3%, DMF 10 μM 5.4%, DMF 100 μM 2.1%, Mito-MES 100 nM 2.4%, Mito-MES 100 nM+DMF 10 μM 0.9%, Mito-MES 100 nM+DMF 100 μM 5.9%) or with brusatol and/or Mito-MES (Mito-MES 1000 nM 2.9%, Brusatol 250 nM 39.9%, Mito-MES 1000 nM+Brusatol 250 nM 17.4%). Pretreatment with Brusatol reduced protein levels of Nrf2 and increased protein levels of Keap in infected and uninfected Calu3 cells. Pretreatment of Calu3 with 0.25 μM Brusatol increased the HO-1 protein levels in SARS-CoV-2 infected cells and did not have an impact on the effect of Mito-MES on HO-1 protein levels. Both 0.25 μM brusatol and 100 μM DMF but not 10 μM DMF were cytotoxic, as determined by XTT cell cytotoxicity assay, in similarly treated uninfected cultures across the dose range (FIG. 5-e). Collectively, the data herein suggests that the antiviral activity of Mito-MES against SARS-CoV-2 is partially mediated through the Nrf2 pathway which may not be reflected by the total cellular protein levels of Nrf2 and Keap and is not mediated by the HO-1 protein.

The impact of Mito-MES on cellular apoptosis associated with SARS-CoV-2 infection was assessed. Immunofluorescence staining showed that Mito-MES reduced SARS-CoV-2-induced increase in cleavage of caspase 3, a key regulator of apoptosis in both Vero E6 and Calu3 cells. Flow cytometry experiments confirmed that Mito-MES attenuated SARS-CoV-2-induced increase in both the percent of infected cells that were positive for cleaved caspase 3 and the median fluorescence intensity (MFI) of cleaved caspase 3 per cell in both Vero and Calu3 cells.

### Mito-MES Shows In Vivo Antiviral Efficacy in Mouse Model of SARS-CoV-2 Infection

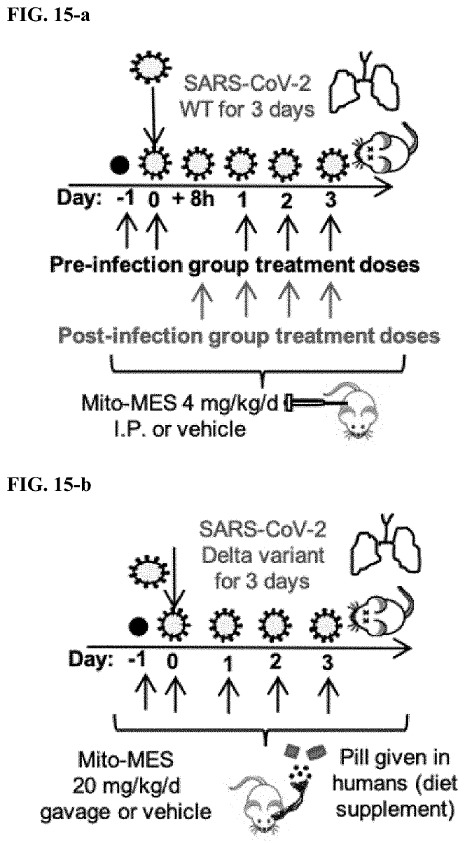

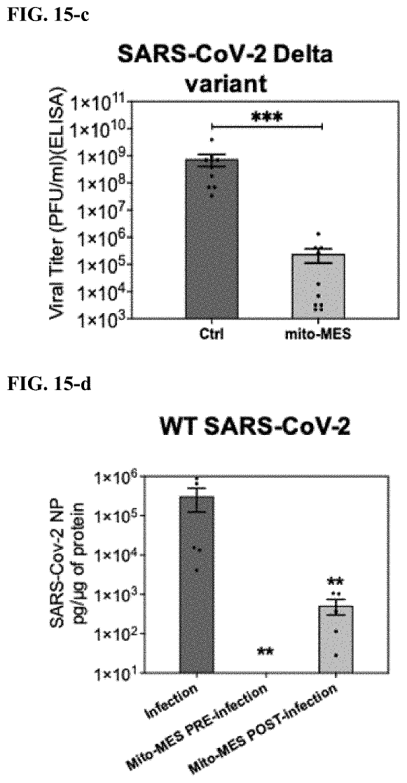

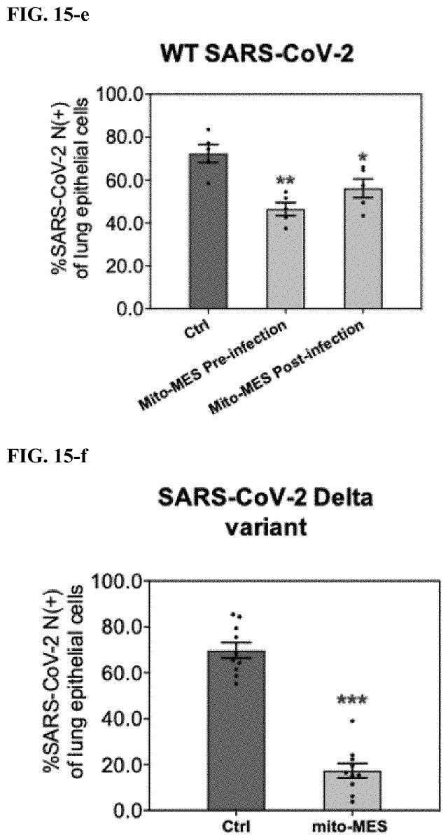

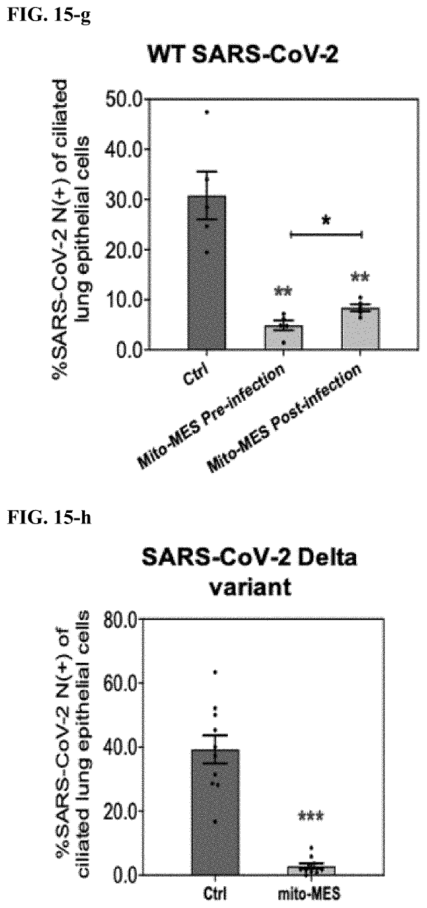

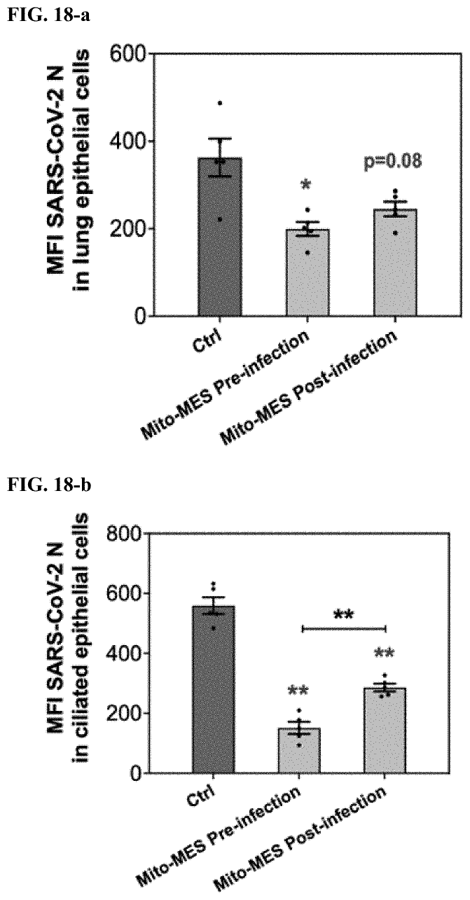

Doses of Mito-MES in mice of 4 mg/kg/day achieve good lung tissue penetration and have been translated to oral doses in humans. Therefore, the in vivo efficacy of Mito-MES to reduce viral titers and viral protein levels in the lung was tested in an established animal model of SARS-CoV-2 infection, the K18-hACE2 mice. As a proof-of-principle in vivo experiment and to ensure adequate Mito-MES levels in the murine lung tissue at the time of SARS-CoV-2 infection, prophylactic dosing of 4 mg/kg Mito-MES was performed 24 hours before SARS-CoV-2 infection that was given intraperitoneal (i.p.) once per day for 3 more days (FIG. 15-a). There was a reduction of nearly 2 log units in SARS-CoV-2 viral titers in the lungs of the Mito-MES group relative to the vehicle control group (FIG. 7-a, FIG. 7-b), which is similar anti-SARS-CoV-2 effect of remdesivir in K18-hACE2 mice. In the lungs of the SARS-CoV-2 infected mice that were treated with 4 mg/kg Mito-MES 8 hours after SARS-CoV-2 infection, there was a reduction of 1.5 log units in SARS-CoV-2 viral titers relative to the vehicle control group (FIG. 7-a, FIG. 7-b). Whether oral Mito-MES obtained from the actual capsule given to humans as diet supplement has in vivo antiviral activity against the Delta variant was assessed (FIG. 15-b). Mito-MES given in SARS-CoV-2 infected K18-hACE2 mice through oral gavage at concentration 20 mg/kg reduced viral titer by nearly 3 log units relative to the vehicle control group (FIG. 15-c). The anti-SARS-CoV-2 activity of Mito-MES was further confirmed by ELISA that determined the protein levels of SARS-CoV-2 N protein in lungs of SARS-CoV-2 infected K18-hACE2 mice (FIG. 15-d). Protein levels of SARS-CoV-2 N protein among different lung cell subtypes in murine lungs after 3 days post-infection (dpi), when viral replication in SARS-CoV-2 infected K18-hACE2 mice is the highest. Mito-MES had anti-SARS-CoV-2 activity in EPCAM(+) lung epithelial cells (FIG. 15-e, FIG. 15-f, FIG. 18-a), FOXJ1(+) ciliated lung epithelial cells (FIG. 15-g, FIG. 15-h; FIG. 18-b), and alveolar type 1 (AT1) and alveolar type 2 (AT2) endothelial cells, and its activity was higher in FOXJ1(+) ciliated lung epithelial cells (FIG. 15-g, FIG. 15-h; FIG. 18-b). Taken together, these experiments show that Mito-MES treatment reduces the replication of SARS-CoV-2 by at least 2 orders of magnitude and has compelling potential for clinical efficacy for the treatment of COVID-19.

Mito-MES Attenuates Apoptosis and Release of Proinflammatory Cytokines Associated with SARS-CoV-2 Infection in Epithelial Cells.

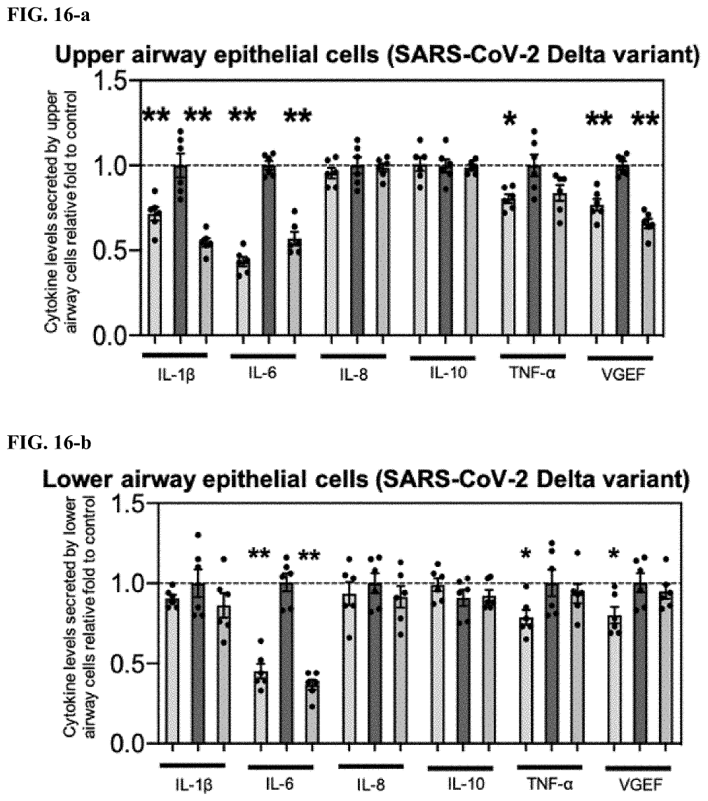

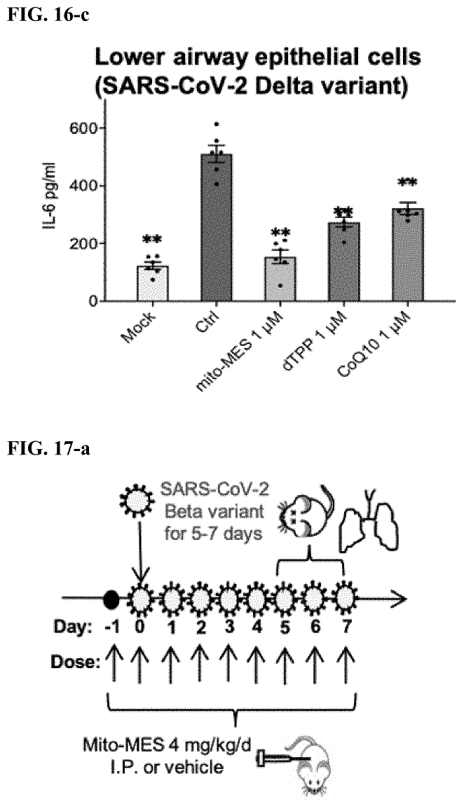

The impact of Mito-MES on inflammatory cellular responses associated with SARS-CoV-2 infection was determined. Using ELISA and Luminex immunoassays, it was found that SARS-CoV-2 infection increased release of IL-6 but did not impact secretion of IL-8, IL-10 and TNF-α in cell culture supernatants of SARS-CoV-2 infected Calu3 (FIG. 5-f, FIG. 5-g), upper (FIG. 16-a) and lower (FIG. 16-b) airway respiratory epithelium ALI cultures. Mito-MES attenuated SARS-CoV-2-induced increase in secretion of IL-6 by Calu3 (FIG. 5-f, FIG. 5-g) cells, upper (FIG. 16-a) and lower (FIG. 16-b) airway respiratory epithelium ALI cultures. Mito-MES also reduced secretion of IL-10 in cell culture supernatants of SARS-CoV-2 infected upper (FIG. 16-a) and lower (FIG. 16-b) airway respiratory epithelium ALI cultures (FIG. 5-f). Thus, Mito-MES attenuates ROS, activation of inflammasome, NFκB signaling that collectively drive a cytokine storm, release of IL-1β, IL-6, and ultimately lung damage caused by coronavirus infections.

### The Anti-Inflammatory Activity of Mito-MES Against SARS-CoV-2 Infected Epithelial Cells is Mediated Through its Antioxidant Properties and the TPP Moiety.

The antiviral and anti-inflammatory activities of Mito-MES against SARS-CoV-2 is mediated through the NrF2 pathway. Pretreatment of Calu3 cultures with 10 μM of the Nrf2 agonist DMF for 24 hours resulted in reduction in subsequent release of IL-6 to the cell supernatant from infected cells at 48 hpi as measured by immunoassays (FIG. 5-h). Compared to treatment of Calu3 with 1000 nM Mito-MES alone, treatment of Calu3 with 10 or 100 μM DMF in combination with Mito-MES for 72 hours resulted in an additive reduction in release of IL-6 to the cell supernatant from SARS-CoV-2 infected cells (FIG. 5-h). Pretreatment of Calu3 with 0.25 μM of the NRF2 inhibitor brusatol significantly increased subsequent release of IL-6 to the cell supernatant from infected cells (FIG. 5-i) and inhibited the anti-inflammatory effect of Mito-MES (FIG. 5-i). Notably, the TPP moiety and the mitochondrial antioxidant CoQ10 also reduced subsequent release of IL-6 to the cell supernatant from SARS-CoV-2 infected ALI cultures (FIG. 16-c). Collectively, these data suggest that Mito-MES has anti-inflammatory effects on SARS-CoV-2 infected epithelial cells through its antioxidant properties (Nrf2 agonist, Co10 moiety, antioxidant against mito-ROS) and the TPP moiety. In conclusion, these results show that, Mito-MES not only has antiviral activity against SARS-CoV-2 but also attenuates inflammatory responses of the infected epithelial cells that drive severe lung injury in COVID-19.

### Mito-MES Shows In Vivo Anti-Inflammatory Efficacy in Mouse Model of SARS-CoV-2 Infection

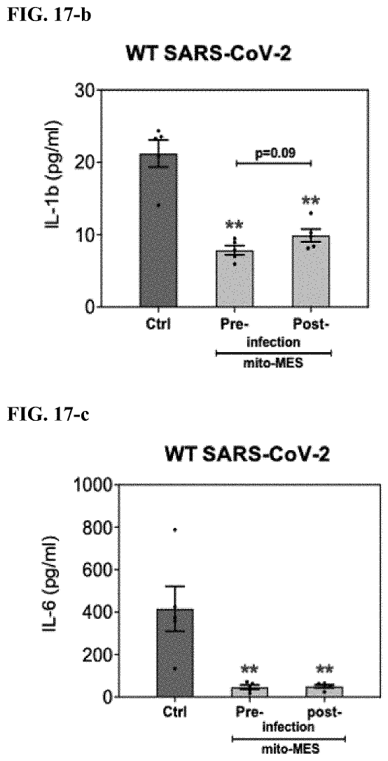

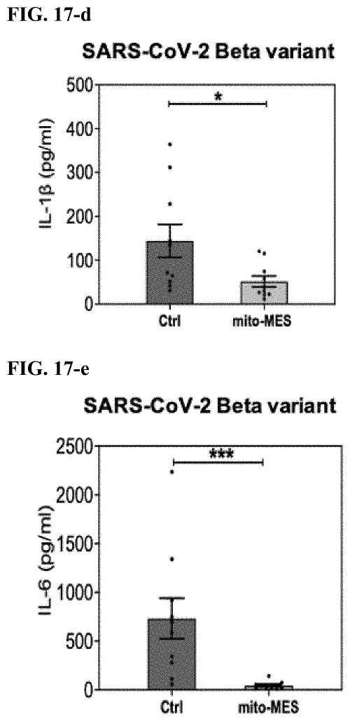

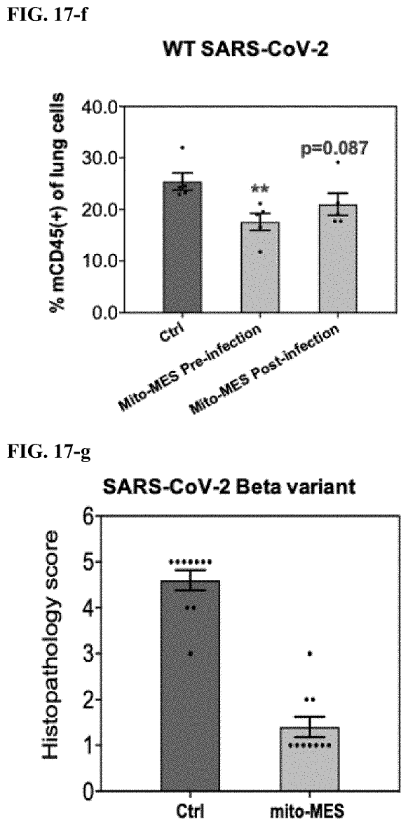

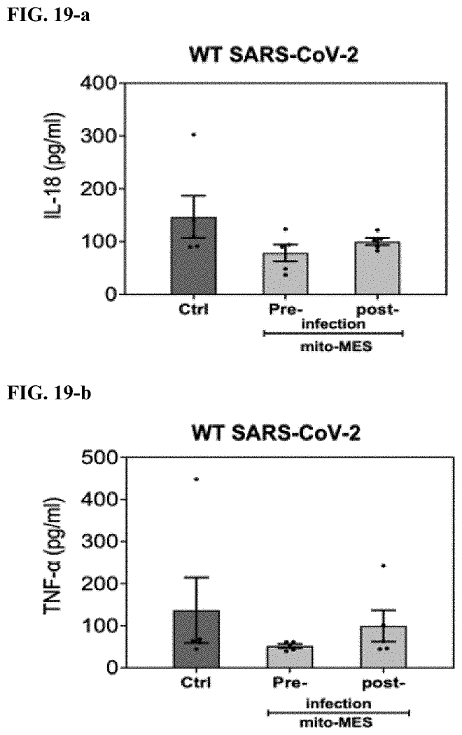

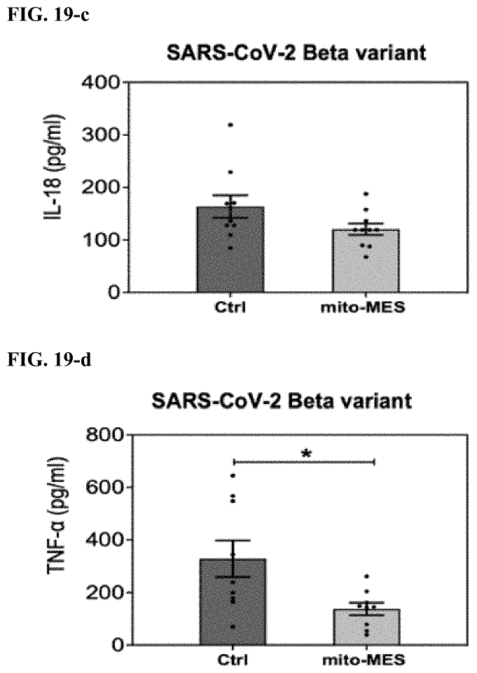

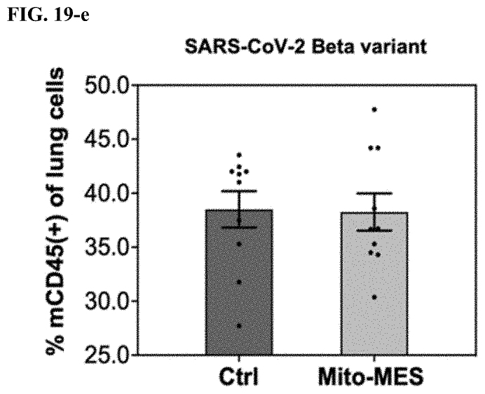

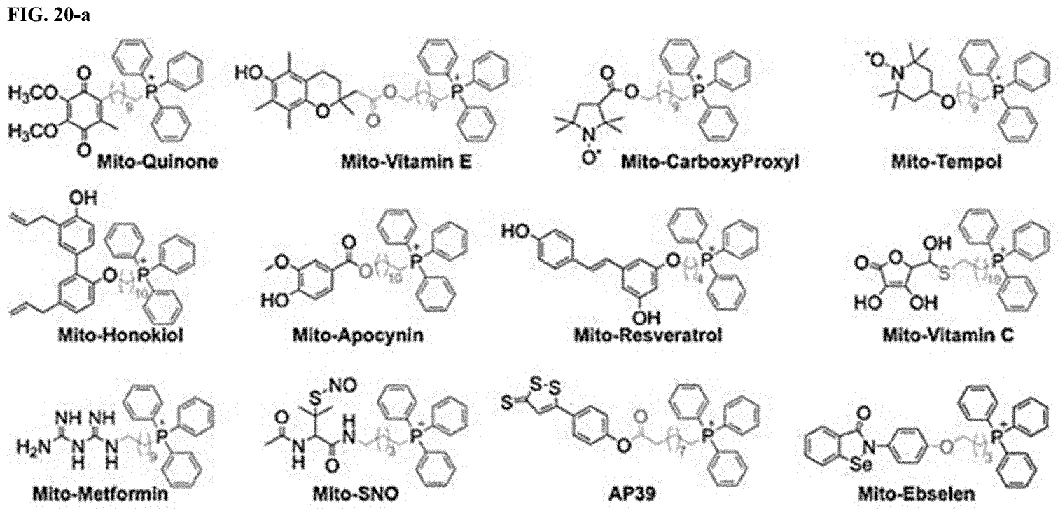

The in vivo anti-inflammatory efficacy of 4 mg/kg intraperitoneous daily dosage of Mito-MES was tested in K18-hACE2 mice infected with the SARS-CoV-2 wild type (WT) (cohort A) as early as 3 dpi (when lung inflammation is the lowest and viral replication is the highest) and in K18-hACE2 mice infected with the Beta variant (cohort B), after 5-7 dpi, when lung inflammation is the highest (FIG. 17-a). Using Luminex immunoassays, there was a reduction of at least 2 orders of magnitude in IL-lb (FIG. 17-b) and IL-6 (FIG. 17-c) in the lungs of the Mito-MES treated SARS-CoV-2 infected relative to the vehicle treated group as early as 3 dpi (FIG. 17-b, FIG. 17-c). The in vivo anti-inflammatory activity (reduction in IL-lb, IL-6, TNFa) of Mito-MES in the lung after 5-7 dpi was confirmed in mice from cohort B infected with the SARS-CoV-2 Beta Variant (FIG. 17-d, FIG. 17-e, FIG. 19-d). Mito-MES did not change protein levels of IL-18 in SARS-CoV-2 infected K18-hACE2 mice (FIG. 19-a, FIG. 19-c) and levels of TNFa in WT SARS-CoV-2 infected K18-hACE2 mice (FIG. 19-b). To fully characterize the anti-inflammatory activity of Mito-MES at the cell level, infiltration of different immune cell subtypes in murine lungs was determined using flow cytometry. Mito-MES reduced frequency of CD45+ immune as early as 3 dpi in mice infected with the WT SARS-CoV-2 (FIG. 17-f). Mito-MES also reduced frequency of NK cells in murine lungs of mice infected with the WT SARS-CoV-2 at 3 dpi and in lungs of mice infected with the SARS-CoV-2 Beta variant at 5-7 dpi. Mito-MES did not alter frequency of neutrophils, macrophages, myeloid dendritic cells (DCs), lymphoid DCs, T cells, and B cells. Immunofluorescence analysis showed that Mito-MES inhibited SARS-CoV-2-associated increase in cleaved caspase 3, a marker of tissue apoptosis and damage, in K18-hACE2 mice infected with Beta variant. Histopathology analysis also showed a reduction of lung inflammation and tissue damage in mito-MES treated mice over vehicle-treated mice at day 5-7 after infection (FIG. 17-g). Taken together, these experiments show that Mito-MES treatment has in vivo anti-inflammatory effect that is seen at both early and late stages of SARS-CoV-2 infection and has good therapeutic efficacy in COVID-19.

### Summary

The SARS-CoV-2 pandemic necessitates the development of antiviral and anti-inflammatory therapeutics that can be rapidly moved into the clinic. Herein, we demonstrate that Mito-MES has in vitro and in vivo antiviral, antiapoptotic and anti-inflammatory effects on SARS-CoV-2 infected epithelial cells. Unlike vaccines, attenuation of detrimental host responses that propagate viral replication may be efficacious even in the setting of mutant strains of SARS-CoV-2 to which viral-targeted therapeutics and vaccines may be less effective. Mito-MES had nanomolar potency against the Beta and Delta variant as well as the MHV.

The antiviral effect of Mito-MES against SARS-CoV-2 in epithelial cells is mediated partially through its antioxidant properties (reduces mito-ROS and induces the Nrf2 pathway) and through the hydrophobic TPP cation that integrates into cellular membranes that are important for SARS-CoV-2 replication. Mito-MES had more potent antiviral activity in interferon competent epithelial cells compared to interferon deficient Vero E6 cells and it induced mitochondrial mediators of IFN-I responses (TOM70 and MX1) that have an important role in host cellular responses against SARS-CoV-2. The anti-inflammatory effect of Mito-MES was mostly seen against IL-6 and NK cell infiltration in SARS-CoV-2 infected mouse lungs. Importantly, Mito-MES at nanomolar concentrations had additive antiviral and anti-inflammatory activity together with DMF which is currently used as an anti-inflammatory drug in relapsing-remitting multiple sclerosis. Thus, the combination of an FDA-approved drug (DMF) with Mito-MES could easily be repurposed and tested in clinical trials to establish a potent novel combined antiviral and anti-inflammatory therapeutic strategy in COVID-19 patients. The data herein indicates that Mito-MES may be an effective COVID-19 therapeutic.

Although host-targeted antivirals may be toxic, the safety profile of Mito-MES is well established in humans. Mito-MES is currently available as a dietary supplement (10 mg orally daily) and its safety for up to one year in doses as high as 80 mg orally daily has been validated in independent clinical trials for oxidative damage-related diseases such as Parkinson's, hepatitis C, as well as vascular dysfunction. Thus, Mito-MES could easily be repurposed and tested in clinical trials as a small molecule inhibitor of SARS-CoV-2 replication and inflammation-induced pathology for outpatient treatment of mild to moderate acute COVID-19. Given its immediate availability in humans, Mito-MES could also be used for post-exposure prophylaxis against SARS-CoV-2 in high-risk exposures.

To date, there is no safe, efficacious oral antiviral that is potent against SARS-CoV-2 variants and has anti-inflammatory activity, that can be administered to subjects long term (i.e., for an indefinite period of time). Mito-MES, however, may be chronically administered to subjects. Therefore, in some embodiments, Mito-MES is chronically administered a subject.

In some embodiments, Mito-MES is chronically administered prophylactically to a subject. In some embodiments, the subject is “in need of” treatment with Mito-MES. In some embodiments, a subject “in need of” treatment with Mito-MES is unvaccinated against SARS-CoV-2. In some embodiments, a subject “in need of” treatment with Mito-MES is one who is immunocompromised. In some embodiments, a subject “in need of” treatment with Mito-MES is one who has a history of one or more breakthrough infections (i.e., one or more infections by SARS-CoV-2, despite being vaccinated thereagainst). In some embodiments, a subject “in need of” treatment with Mito-MES is one who is repeatedly or continuously exposed to SARS-CoV-2.

In some embodiments, Mito-MES is administered to a subject to treat, inhibit, and/or reduce Post-Acute COVID-19 Syndrome (PACS) in the subject.

### Triphenylphosphonium (TPP) Compounds as Therapeutics

As provided herein, the triphenylphosphonium (TPP) moiety itself exhibits anti-viral activity against SARS-CoV-2, especially the Delta variant, and therapeutic efficacy against symptoms and complications of coronavirus diseases (e.g., COVID-19).

Specifically, viral titer assays in both hACE2 HEK293T cells infected with Beta variant (FIG. 13-g) and ALI HAE cultures infected with Delta variant (FIG. 13-h) show that 1000 nM TPP was not cytotoxic but had potent antiviral activity against both the SARS-CoV-2 Beta stain (FIG. 13-g) as well as the Delta (B.1.617.2) SARS-CoV-2 variant (FIG. 13-h). Additionally, the TPP moiety also reduced subsequent release of IL-6 to the cell supernatant from SARS-CoV-2 infected ALI cultures (FIG. 16-c). These results show that the TPP moiety not only has antiviral activity against SARS-CoV-2 but also attenuates inflammatory responses of the infected epithelial cells that drive severe lung injury in COVID-19.

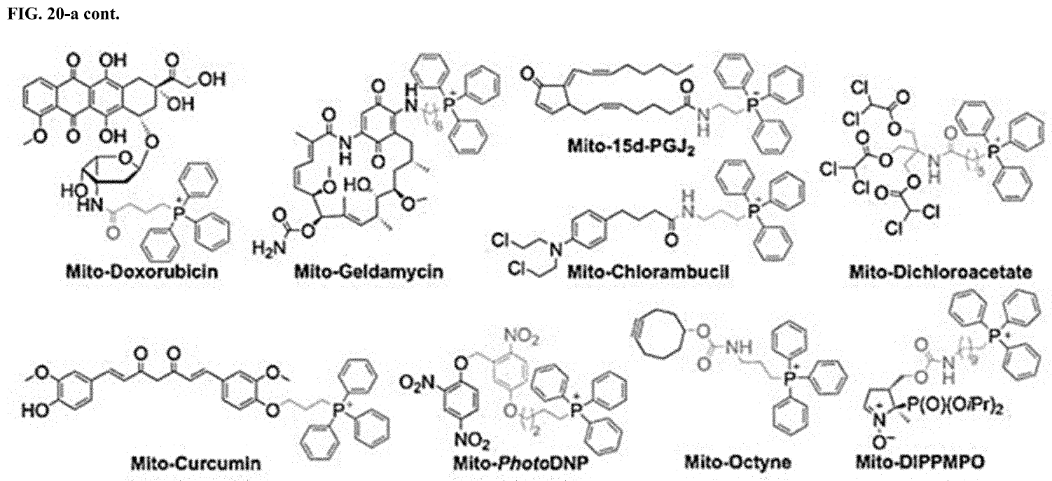

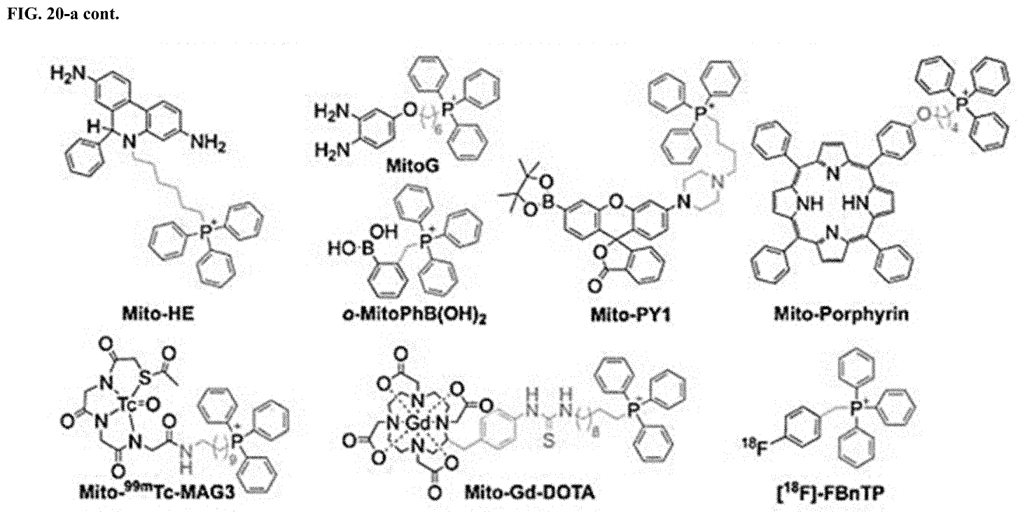

Therefore, in some embodiments, one or more TPP Compounds may be used to treat, prevent, inhibit, and/or reduce (a) an infection by, (b) an inflammatory response caused by, or (c) apoptosis caused by a coronavirus in a cell or a subject, which comprises administering one or more TPP Compounds to the cell or the subject. In some embodiments, the one or more TPP Compounds is administered without mitoquin. In some embodiments, the one or more TPP Compounds is a TPP Hydrocarbon. In some embodiments, the one or more TPP Compounds is a TPP Conjugate. In some embodiments, the one or more TPP Compounds is selected from the group of compounds set forth in FIG. 20-a. In some embodiments, the one or more TPP Compounds is Mito-MES or dTPP.

In some embodiments, one or more antioxidants, one or more ApoA-I mimetic peptides, and/or one or more Nrf2 agonists may be co-administered with the one or more TPP Compounds. In some embodiments, (a) the one or more TPP Compounds, and (b) the one or more antioxidants, the one or more ApoA-I mimetic peptides, and/or the one or more Nrf2 agonists are administered in a molar ratio of (a) to (b) of about a 1:1. In some embodiments, the one or more TPP Compounds, and the one or more antioxidants, the one or more ApoA-I mimetic peptides, and/or the one or more Nrf2 agonists are administered in a lipid carrier.

In some embodiments, one or more “bioactive moieties” may be co-administered with the one or more TPP Compounds. In some embodiments, the one or more TPP Compounds and the one or more bioactive moieties are administered in a molar ratio of about a 1:1. In some embodiments, the one or more TPP Compounds and the one or more bioactive moieties are administered in a lipid carrier. In some embodiments, the one or more bioactive moieties is an antioxidant (e.g., tocopherol, ubiquinone and derivatives thereof, thymoquinone, plastoquinone, etc.). In some embodiments, the one or more bioactive moieties is an Nrf2 agonist (curcumin, resveratrol, DMF, etc.). In some embodiments, the one or more bioactive moieties is ubiquinone or a derivatives thereof, Vitamin E, CarboxyProxyl, Tempol, Honokiol, Apocynin, Resveratrol, Vitamin C, Metformin, S-nitrosothiol or a compound containing S-nitrosothiol as part of its chemical structure, dithiolethione, Ebselen, Doxorubicin, Geldamycin, 15d-PGJ2, Dichloroacetate, Chlorambucil, Curcumin, PhotoDNP, Octyne, DIPPMPO, dihydroethidium, phenylboronic acid, a dipolar 1,3,6,8-tetrasubstituted pyrene-based compound, porphyrin, 99mTc-MAG3, or Gd-DOTA.

### Conclusion

As disclosed herein, the mitochondrial targeted antioxidant, Mito-MES, attenuates SARS-CoV-2 replication in epithelial cells, induces release of proinflammatory cytokines in epithelial cells, attenuates SARS-CoV-2 induced release of proinflammatory cytokines by infected epithelial cells, induces programmed cell death in infected epithelial cells, induces mitochondrial oxidative stress, exerts antiviral activity through the interferon system, and attenuates SARS-CoV-2 induced alterations in cell signaling pathways involving the cross talk between cellular host responses and mitochondria at the nM level. Mito-MES also attenuates SARS-CoV-2 replication in lung tissue of infected mice.

Mito-MES not only treats Covid-19 (via its anti-inflammatory and antiviral activities) but it prevents or inhibits the establishment of a SARS-CoV-2 infection when administered prophylactically pre- or post-exposure to SARS-CoV-2. Particularly, the experiments herein indicate that a plasma concentration of about 2 ng/ml of mitoquin in subjects is protective against infection by SARS-CoV-2 at exposure levels typical of regular and repeated close familial contact. That is, administration of 20 mg/day of Mito-MES results in in vivo levels that prevent, inhibit, and/or reduce the development of infection by SARS-CoV-2 in subjects.

Additionally, as disclosed herein, the ApoA-I mimetic peptide, 4F, prevents, inhibits, and/or reduces infection by, replication of, apoptosis caused by, and inflammation caused by SARS-CoV-2. The combination of Mito-MES+4F is synergistic and the addition of the Nrf2 agonist, DMF, (i.e Mito-MES+4F+DMF) is further synergistic over Mito-MES+4F.

Therefore, in some embodiments, one or more mitochondrial targeted antioxidants and/or one or more ApoA-I mimetic peptides is administered to a subject to prevent, inhibit, and/or treat the subject for an infection by a coronavirus, e.g., SARS-CoV-2. In some embodiments, one or more mitochondrial targeted antioxidants and/or one or more ApoA-I mimetic peptides is administered to a subject to prevent, inhibit, reduce, and/or treat injury to lung tissue caused by a coronavirus, such as SARS-CoV-2, in the subject. In some embodiments, one or more mitochondrial targeted antioxidant and/or one or more ApoA-I mimetic peptides is administered to a subject to prevent, inhibit, reduce, and/or treat a coronavirus disease, such as COVID-19, in a subject. In some embodiments, the coronavirus disease is severe COVID-19, post-acute COVID-19 syndrome (PACS), or chronic Covid-19 syndrome (CCS). In some embodiments, one or more mitochondrial targeted antioxidants, such as Mito-MES, is administered pre- or post-exposure to a subject to prevent, inhibit, and/or reduce the likelihood that the subject becomes infected with a coronavirus, such as SARS-CoV-2, when exposed thereto. In some embodiments, one or more Nrf2 agonists, such as DMF, may be administered in combination with the one or more mitochondrial targeted antioxidants and/or the one or more ApoA-I mimetic peptides. In some embodiments, one or more mitochondrial targeted antioxidants+one or more ApoA-I mimetic peptides are administered. In some embodiments, one or more mitochondrial targeted antioxidants+one or more ApoA-I mimetic peptides+one or more Nrf2 agonists are administered. In some embodiments, Mito-MES+4F are administered. In some embodiments, Mito-MES+4F+DMF are administered.

The administration may be before, during, and/or after exposure or likely exposure to the coronavirus. Preferably, post-exposure administration begins within 4 days or less, preferably 3 days or less, more preferably 2 days or less, even more preferably 1 day or less, and most preferably 12 hours or less from the time of exposure or likely to the coronavirus. The administered amount may be a single dose or multiple doses administered over a period of time. The period of time of administration may be over the course of continuous or intermittent exposure to the coronavirus.

In mice, Mito-MES accumulates in tissues including mitochondria-enriched epithelial tissues like liver and kidney at 50-700 pmol/g (wet weight) following >10 days of oral Mito-MES supplementation. Peak plasma concentration occurs within 1 h of oral administration and then slowly declines over time with an elimination half-life based on post 4 h data of about 14 h. Mitoquin is rapidly distributed to tissues, with a brain:plasma ratio of about 1:10 after 10 minutes and levels of mitoquin in brain tissue are at least 3 times lower than that in epithelial tissues (kidney and liver). Thus, plasma levels at least, partially reflect the mitoquin levels in epithelial tissues in vivo.

In humans, a single 80 mg oral dose of Mito-MES results in a maximal plasma concentration of 33 ng/mL about 1 h after administration and a single 40 mg oral dose of Mito-MES results in a plasma concentration of about 2 ng/ml after 24 h. Thus, a single dose of Mito-MES can result in nanomolar concentrations in plasma and tissues shortly after administration, e.g., within about 1 h after administration. Consequently, a single oral dose of Mito-MES (about 0.33 mg/kg weight of the subject, e.g., about 20 mg for the average human adult) can result in plasma and tissue (e.g., the upper respiratory mucosa) levels that are sufficient to prevent, inhibit, and/or reduce the likelihood that a subject becomes infected with SARS-CoV-2 when exposed thereto.

Chronic supplementation of Mito-MES at about 40-80 mg/day results in an average elevation of fasting plasma concentrations of about 5 ng/mL after about 10 days. Mito-MES has been administered to human subjects at 90 mg/day for 8 weeks. Doses as high as 230 mg/kg/day are non-mutagenic and non-toxic in mice. Using the U.S. FDA recommended surface area conversion factor, this is equivalent to a dose of about 1,119 mg/day for an average 60 kg human (i.e., about 18.7 mg/kg/day), which is about 60-fold higher than the doses administered to A1 and A2 (i.e., 20 mg/day=about 0.33 mg/kg/day). Therefore, Mito-MES may be administered to humans at doses up to at least 80 mg/day for at least a year without any serious adverse events. Preferably, the amount of the dose or doses is 20 mg/day or about 0.33 mg/day per kg weight of the subject being treated. In some embodiments, the amount of the one or more mitochondrial targeted antioxidants administered to the subject is about 0.05 mg/kg to about 15 mg/kg, preferably about 0.2 mg/kg to about 1.5 mg/kg, or more preferably about 0.3 mg/kg to about 0.7 mg/kg weight of the subject per day. In some embodiments, the amount of the one or more mitochondrial targeted antioxidants administered to the subject is about 1-1000 mg, 5-100 mg, 10-80 mg, or 20-40 mg, and preferably about 20 mg per day. In some embodiments, the amount of the one or more mitochondrial targeted antioxidants administered to the subject is 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg per day. In some embodiments, the amount of the one or more ApoA-I mimetic peptides administered to the subject is about 0.02-15 mg/kg, about 0.15-10 mg/kg, about 1.5-10 mg/kg, about 1.0-2.0 mg/kg, about 0.01-7.5 mg/kg, about 0.05-5.0 mg/kg, about 0.75-5.0 mg/kg, or about 0.5-1.0 mg/kg weight of the subject per day. In some embodiments, the amount of the one or more ApoA-I mimetic peptides administered to the subject is about 1-500 mg, about 10-500 mg, about 100-500 mg, about 1-250 mg, about 10-250 mg, or about 100-250 mg per day. In some embodiments, the amount of the one or more Nrf2 agonists that is administered to the subject is about 0.02-8.0 mg/kg, about 0.15-8.0 mg/kg, about 4.0-8.0 mg/kg, about 0.01-4.0 mg/kg, about 0.1-4.0 mg/kg, or about 2.0-4.0 mg/kg weight of the subject per day. In some embodiments, the amount of the one or more Nrf2 agonists that is administered to the subject is about 1-480 mg, about 10-480 mg, about 240-480 mg, about 0.5-240 mg, about 5-240 mg, or about 120-240 mg per day. In some embodiments, the amount of the Mito-MES that is administered to a subject in order to prevent, inhibit, and/or reduce an infection by or replication of a coronavirus, e.g., SARS-CoV-2, in the subject is one that causes a concentration of about 2 ng/ml or higher of mitoquin during the period the subject is exposed or likely exposed to the coronavirus or during the period the subject is at risk of developing an infection by the coronavirus.

In some embodiments, administration of the one or more mitochondrial targeted antioxidants and/or the one or more ApoA-I mimetic peptides begins prior to an expected or possible exposure to the coronavirus. In some embodiments, administration of the one or more mitochondrial targeted antioxidants and/or the one or more ApoA-I mimetic peptides begins at the time of or within about 96 hours, preferably within about 72 hours, more preferably within about 48 hours, even more preferably within about 24 hours, and most preferably within about 12 hours, from a suspected or confirmed exposure to the coronavirus. In some embodiments, the administration is for at least about 1, 2, 3, 4, 5, 6, 7, 8, 9, or 10 days, preferably about 5-7 days from the suspected or confirmed exposure to the coronavirus.

In some embodiments, the one or more the mitochondrial targeted antioxidants and/or the one or more ApoA-I mimetic peptides are administered to subjects “in need thereof”. As used herein, subjects “in need of” include those who are likely to be exposed or have been exposed to a coronavirus and those who belong to “high-risk” groups (e.g., elderly, those suffering from comorbidities, immunocompromised subjects, subjects unvaccinated against the given coronavirus, first responders, and health care workers).

As used herein, a “coronavirus disease” refers to a disease caused by infection by a virus belonging to the family Coronaviridae. That is, a coronavirus disease refers to the adverse physiological events that result from an infection by a coronavirus, such as post-infectious chronic sequalae and long-term cardiovascular effects, organ and tissue injury and damage, adverse inflammation (e.g., neuroinflammation, intestinal inflammation, lung inflammation), and the like. In some embodiments, the coronavirus disease is COVID-19, which includes acute COVID-19, severe COVID-19, post-acute COVID-19 syndrome (PACS), and chronic Covid-19 syndrome (CCS). As used herein, a “coronavirus” refers to a virus belonging to the family Coronaviridae. In some embodiments, the coronavirus belongs to the subfamily Orthocoronavirinae. In some embodiments, the coronavirus belongs to the genera Alphacoronavirus or Betacoronavirus. In some embodiments, the coronavirus is a human coronavirus. In some embodiments the coronavirus is HCoV-229E, HCoV-NL63, HCoV-0C43, HCoV-HKU1, SARS-CoV, MERS-CoV, or SARS-CoV-2, preferably SARS-CoV, MERS-CoV, or SARS-CoV-2, more preferably SARS-CoV-2. As used herein, “SARS-CoV-2” includes the original strain and its variants (e.g., Alpha (B.1.1.7, Q lineages), Beta (B.1.351), Gamma (P.1), Epsilon (B.1.427, B.1.429), Eta (B.1.525), Iota (B.1.526), Kappa (B.1.617.1), 1.617.3, Mu (B.1.621, B.1.621.1), Zeta (P.2), Delta (B.1.617.2, AY lineages), and Omicron (B.1.1.529, BA lineages), and descendant lineages thereof).

Symptoms and complications of a coronavirus disease includes cytokine storm, adverse inflammatory reactions, acute respiratory distress syndrome (ARDS), sepsis, and multiple organ dysfunction or failure (MOF).

As used herein, a “mitochondrial targeted antioxidant” refers to an antioxidant that scavenges reactive oxygen species in mitochondria (“mito-ROS”). Mitochondrial targeted antioxidants include mitoquinone mesylate (10-(4,5-dimethoxy-2-methyl-3,6-dioxo-1,4-cyclohexadienyl) decyl triphenylphosphonium methanesulfonate, which is commercially available as “Mito-Q®”), derivatives thereof (e.g., the dihydroxy form—mitoquinol mesylate, i.e 10-(4,5-dimethoxy-2-methyl-3,6-dihydroxy-1,4-cyclohexadienyl) decyl triphenylphosphonium methanesulfonate), mitoquinone and salts thereof (other than methanesulfonate), mitoquinol and salts thereof (other than methanesulfonate), etc.), other mitochondrial targeted antioxidants in the art such as SkQ1 (Mitotech, S.A.), Elamipretide (Stealth BioTherapeutics), Mito-TEMPO (CAS 1569257-94-8), and those disclosed in the following patents and publications: U.S. Pat. Nos. 8,518,915; 9,192,676; 9,328,130; 9,388,156; US20070161609; US20070225255; US20080161267; US20100168198; US20160200749; US20180305328; US20190248816; US20190330249; US20190374558; WO2005019232; WO2006005759; WO2007046729; WO2008145116; WO2015063553; WO2017106803; and WO2018162581, which are herein incorporated by reference. As used herein, “Mito-MES” is used to refer to mitoquinone (10-(4,5-dimethoxy-2-methyl-3,6-dioxo-1,4-cyclohexadienyl) decyl triphenylphosphonium) mesylate and/or mitoquinol (10-(4,5-dimethoxy-2-methyl-3,6-dihydroxy-1,4-cyclohexadienyl) decyl triphenylphosphonium) mesylate, and the term “mitoquin” is used to refer to mitoquinone and/or mitoquinol. It should be noted that in vivo mitoquinone is rapidly distributed to tissues and then converted into mitoquinol within cells and either mitoquinone or mitoquinol may be used by cells in vitro. For convenience, the term “Mito-MES” is used throughout the experiments described herein; however, mitoquinone mesylate was used for the in vitro experiments and mitoquinol mesylate was used for the in vivo animal studies, unless specifically indicated otherwise.

As used herein, “ApoA-I mimetic peptides” includes ApoA-I mimetic peptides known in the art. See, e.g., Stoekenbroek, et al. (2015) ApoA-I Mimetics. In: von Eckardstein A., Kardassis D. (eds) High Density Lipoproteins. Handbook of Experimental Pharmacology, vol 224. Springer, Cham.; Navab, et al. (2015) ApoA-I Mimetic Peptides: A Review of the Present Status. In: Anantharamaiah G., Goldberg D. (eds) Apolipoprotein Mimetics in the Management of Human Disease. Adis, Cham.; Islam, et al. (2018) Structural properties of apolipoprotein A-I mimetic peptides that promote ABCA1-dependent cholesterol efflux. Sci Rep 8: 2956; Gou, et al. (2020) Br J Pharmacol. 177(20):4627-4644; Yang, et al. (2019) Respir Res 20: 131; Van Lenten, et al. (2009) Curr Atheroscler Rep 11(1):52-57; and Datta G, et al. (2001) J Lipid Res 42(7): 1096-1104, which including their references cited therein are herein incorporated by reference. ApoA-I mimetic peptides include 4F, D-4F, reverse D-4F, and 6F. ApoA-I mimetic peptides are preferably selected from 4F, D-4F, reverse D-4F, and 6F, more preferably 4F, D-4F, and 6F, and most preferably 4F and D-4F.

Compositions, including pharmaceutical compositions, comprising one or more mitochondrial targeted antioxidants and/or one or more ApoA-I mimetic peptides are contemplated herein. The term “pharmaceutical composition” refers to a composition suitable for pharmaceutical use in a subject. A composition generally comprises an effective amount of an active agent and a diluent and/or carrier. A pharmaceutical composition generally comprises a therapeutically effective amount of an active agent and a pharmaceutically acceptable carrier. In addition to the one or more mitochondrial targeted antioxidants and/or the one or more ApoA-I mimetic peptides, pharmaceutical compositions may include one or more supplementary agents. Examples of suitable supplementary agents include anti-inflammatory agents, antiviral agents, and Nrf2 agonists known in the art. Nrf2 agonists include Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF).

In some embodiments, the compositions comprise one or more mitochondrial targeted antioxidants+one or more ApoA-I mimetic peptides. In some embodiments, the compositions comprise one or more mitochondrial targeted antioxidants+one or more Nrf2 agonists. In some embodiments, the compositions comprise one or more mitochondrial targeted antioxidants+one or more ApoA-I mimetic peptides+one or more Nrf2 agonists. The amount of the one or more mitochondrial targeted antioxidants, one or more ApoA-I mimetic peptides, and/or one or more Nrf2 agonists may be provided in an effective amount or a therapeutically effective amount and/or in a synergistic ratio as provided herein. In some embodiments, the amount of the one or more mitochondrial targeted antioxidants is about 1-1000 mg, 5-100 mg, 10-80 mg, or 20-40 mg, and preferably about 20 mg. In some embodiments, about the amount of the one or more mitochondrial targeted antioxidants is about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about or 200 mg. In some embodiments, the amount of the one or more ApoA-I mimetic peptides is about 1-500 mg, about 10-500 mg, about 100-500 mg, about 1-250 mg, about 10-250 mg, or about 100-250 mg. In some embodiments, the amount of the one or more Nrf2 agonists is about 1-480 mg, about 10-480 mg, about 240-480 mg, about 0.5-240 mg, about 5-240 mg, or about 120-240 mg.

As used herein, an “effective amount” refers to a dosage or amount sufficient to produce a desired result. The desired result may comprise an objective or subjective change as compared to a control in, for example, in vitro assays, and other laboratory experiments. As used herein, a “therapeutically effective amount” refers to an amount that may be used to treat, prevent, or inhibit a given disease or condition in a subject as compared to a control, such as a placebo. Again, the skilled artisan will appreciate that certain factors may influence the amount required to effectively treat a subject, including the degree of the condition or symptom to be treated, previous treatments, the general health and age of the subject, and the like. Nevertheless, effective amounts and therapeutically effective amounts may be readily determined by methods in the art.

The one or more mitochondrial targeted antioxidants and/or the one or more ApoA-I mimetic peptides may be administered, preferably in the form of pharmaceutical compositions, to a subject. Preferably the subject is mammalian, more preferably, the subject is human. Preferred pharmaceutical compositions are those comprising at least one mitochondrial targeted antioxidant and/or at least one ApoA-I mimetic peptide in a therapeutically effective amount and a pharmaceutically acceptable vehicle. In some embodiments, a therapeutically effective amount of a mitochondrial targeted antioxidant, such as Mito-MES, ranges from about 0.05 mg/kg to about 15 mg/kg, preferably about 0.2 mg/kg to about 1.5 mg/kg, or more preferably about 0.3 mg/kg to about 0.7 mg/kg body weight. In some embodiments, a therapeutically effective amount of an ApoA-I mimetic peptide, such as 4F, ranges from about 0.02-15 mg/kg, about 0.15-10 mg/kg, about 1.5-10 mg/kg, about 1.0-2.0 mg/kg, about 0.01-7.5 mg/kg, about 0.05-5.0 mg/kg, about 0.75-5.0 mg/kg, or about 0.5-1.0 mg/kg, preferably about 1.2-2 mg/kg body weight. In some embodiments, a therapeutically effective amount of an Nrf2 agonist, such as DMF, ranges from about 0.02-8.0 mg/kg, about 0.15-8.0 mg/kg, about 4.0-8.0 mg/kg, about 0.01-4.0 mg/kg, about 0.1-4.0 mg/kg, or about 2.0-4.0 mg/kg weight. It should be noted that treatment of a subject with a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions may be formulated for the intended route of delivery, including intravenous, intramuscular, intra peritoneal, subcutaneous, intraocular, intrathecal, intraarticular, intrasynovial, cisternal, intrahepatic, intralesional injection, intracranial injection, infusion, and/or inhaled routes of administration using methods known in the art. Pharmaceutical compositions may include one or more of the following: pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art.

The compositions may be administered to a subject by any suitable route including oral, transdermal, subcutaneous, intranasal, inhalation, intramuscular, and intravascular administration. It will be appreciated that the preferred route of administration and pharmaceutical formulation will vary with the condition and age of the subject, the nature of the condition to be treated, the therapeutic effect desired, and the particular mitochondrial targeted antioxidant and/or ApoA-I mimetic peptide used.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20^{th} ed (2000) Lippincott Williams & Wilkins, Baltimore, Md.

The pharmaceutical compositions may be provided in dosage unit forms. As used herein, a “dosage unit form” refers to physically discrete units suited as unitary dosages for the subject to be treated; each unit containing a predetermined quantity of the one or more mitochondrial targeted antioxidant and/or the one or more ApoA-I mimetic peptides calculated to produce the desired therapeutic effect in association with the required pharmaceutically acceptable carrier. The specification for the dosage unit forms of the invention are dictated by and directly dependent on the unique characteristics of the given mitochondrial targeted antioxidant and/or ApoA-I mimetic peptide and desired therapeutic effect to be achieved, and the limitations inherent in the art of compounding such an active compound for the treatment of individuals.

Toxicity and therapeutic efficacy of mitochondrial targeted antioxidants, ApoA-I mimetic peptides, and compositions thereof can be determined using cell cultures and/or experimental animals and pharmaceutical procedures in the art. For example, one may determine the lethal dose, LC₅₀ (the dose expressed as concentration×exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Mitochondrial targeted antioxidants and ApoA-I mimetic peptides which exhibit large therapeutic indices are preferred. While mitochondrial targeted antioxidants and ApoA-I mimetic peptides that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of one or more mitochondrial targeted antioxidants and/or one or more ApoA-I mimetic peptides can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### Examples

### Materials

Mito-MES was purchased from Cayman Chemical. Decyl triphenyl phosphonium cation (TPP), Remdesivir, Brusatol, and DMSO were purchased from Sigma Aldrich. 4F was synthesized and prepared using methods in the art and then used at 1-100 μM (from 1000× stocks prepared in water). Remdesivir was dissolved in DMSO and was then used at 1 μM.

SARS-CoV-2, isolate 2019-nCoV/USA-WA1/2020 strain, GenBank Accession No. MN985325.1) was obtained from Biodefense and Emerging Infectious (BEI) Resources of National Institute of Allergy and Infectious Diseases (NIAID). K18-hACE2 mice were purchased from JAX laboratories.

Human adenocarcinoma lung epithelial (Calu3) cells (ATCC, HTB-55) and African green monkey kidney epithelial Vero-E6 cells (ATCC, CRL-1586) were maintained at 37° C. and 5% CO2 in Modified Eagle Medium (MEM, Corning) supplemented with 10% Fetal Bovine Serum (FBS), penicillin (100 units/ml), and streptomycin (100 μg/ml). HEK293T cells stably expressing human ACE2 (HEK293-ACE2 cells) were purchased from Genecopoeia and were maintained at 37° C. and 5% CO2 in Modified Eagle Medium (MEM, Corning) supplemented with 10% Fetal Bovine Serum (FBS), and hygromycin.

Human Primary Small Airway Epithelial Cells (HSAECs) ALI cultures: HSAECs were seeded onto collagen coated transwells and grew them in the submerged phase of culture for 4-5 days in PneumaCult Ex Plus media (Stem Cell Technologies) with 500 μL media in the basal chamber and 200 μL media in the apical chamber. ALI cultures were then maintained for 21 days with only 500 μL PneumaCult ALI media (Stem Cell Technologies) in the basal chamber, and media changed every 2 days. Cultures were maintained at 37° C. and 5% CO2.

The following antibodies were obtained from Thermo Fisher Scientific: SARS-CoV-2 N (Cat #PIMA536086), TMPRSS2 (Cat #PA514), FoxJ1/HFH4 Rat anti-Human, Alexa Fluor 594, Nrf2 (Cat #50-553-534), Keap 1 (cat #N271496A647), Goat anti-mouse Alexa Fluor® 546 (Cat #ab96902), The following antibodies were obtained from Cell Signaling Technology (cleaved caspase-3 (Cat #9661, cat ##9602), MAVS (D5A9E) (Cat #18930)), Sino Biologicals (SARS-CoV-2 Spike S (Cat #40150-R007)), GeneTex (SARS-CoV-2 Spike S (Cat #GTX632604)), Novus Biologicals (ACE2 (Cat #NBP2-80035), Biolegend (Brilliant Violet 421™ anti-human CD304 (Neuropilin-1) Antibody, PE/Cyanine7 anti-human CD147 Antibody), Proteintech (Tom70 (Cat #50-13750-1-AP), MX1 (Cat #50-13750-1-AP)), Abcam (Goat anti-rabbit Alexa Fluor® 488 IgG (Cat #ab150077), Goat anti-rabbit DyLight® 650 (Cat #ab96902), LSBio (Heme oxygenase-1 (HO-1) (Cat #LS-C343604-200). Antibodies were conjugated with Mix-n-Stain CF568, CF488, CF594, CF647 Antibody Labeling Kits from Biotium Inc (Hayward, Calif.), as needed according to the manufacturer's protocol.

### SARS-CoV-2 Infection

SARS-CoV-2, isolate 2019-nCoV/USA-WA1/2020 strain, (GenBank Accession No. MN985325.1) was obtained from Biodefense and Emerging Infectious (BEI) Resources of National Institute of Allergy and Infectious Diseases (NIAID). All studies involving live virus were conducted at the UCLA BSL3 high-containment facility with appropriate institutional biosafety approvals. SARS-CoV-2 was passaged once in Vero-E6 cells and viral stocks were aliquoted and stored at −80° C. Virus titer was measured in Vero-E6 cells by TCID50 assay. Cell cultures in 96 well plates were infected with SARS-CoV-2 viral inoculum (MOI of 0.1; 100 μl/well) prepared in media. For mock infection, conditioned media (100 μl/well) alone was added. For infection of Calu3 cells in 96 well plates with the fluorescent reporter a stable mNeonGreen SARS-CoV-2 virus (icSARS-CoV-2-mNG), an MOI of 0.3 and 100 μl/well was used.

RNA Extraction and Quantitative Polymerase Chain Reaction (q-RT-PCR)

Total RNA from infected and mock infected cells was lysed in TRIzol (Invitrogen) and extracted and DNase I treated using the RNeasy Mini Kit (Qiagen 74104) or Direct-zol RNA Miniprep kit (Zymo Research) according to the manufacturer's instructions. RNA was quantified using a NanoDrop Spectrophotometer (ThermoFisher). RNA was reverse transcribed into cDNA using oligo d(T) primers using SuperScript II Reverse Transcriptase (Thermo Fisher).

Quantitative real-time PCR was performed using Green qPCR Master Mix (Thermo Fisher) and primers specific for SARS-CoV-2 as well as GADPH transcripts. All qRT-PCR reactions were performed using BIO-RAD CFX96 Touch Real-Time PCR Detection System on 96-well plates. Each 20 μL reaction mixture contained 10 μL of 2×SYBR Green RT-PCR Master Mix (Thermo), 4.2 μL of RNase-free water, 0.4 μL each of 10 μmol/L forward and reverse primer and 5 μL of cDNA as template. The reactions were incubated at 45° C. for 10 min for reverse transcription, 95° C. for 2 min, followed by 40 cycles of 95° C. for 15 s and 60° C. for 60 s. Gene expression fold change was calculated with the Delta-delta-cycle threshold (DDCt) method using Microsoft Excel was determined relative to mock infected samples. Viral RNA levels were normalized to GADPH as an endogenous control and depicted as fold change over mock infected samples. Briefly, DDCt=DCt (SARS-CoV-2-infected)−DCt(mock control) with DCt=Ct(gene-of-interest)−Ct(housekeeping-gene-GADPH). The fold change for each gene is calculated as 2\hat{ }-DDCt. Error bars indicate the standard error of means from three biological replicates.

### Imaging and Immunofluorescence

After 24-48 h of SARS-CoV-2 infection (or mock), live cell images were obtained by bright field microscope (for determination of cytopathic effects (CPE) (Leica DMIL LED). For immunofluorescence, separate wells of cells were fixed with 4% paraformaldehyde in phosphate-buffered saline (PBS) for 20 min. The fixed samples were then permeabilized and blocked for 1 h in a “blocking solution” containing PBS with 2% bovine serum albumin, 5% donkey serum, 5% goat serum, and 0.3% Triton X-100. Primary antibodies were diluted in the blocking solution and added to samples overnight at 4° C. The following antibodies and dilutions were used: Spike S (1:100, Sino Biologicals Cat #40150-R007). Samples were then rinsed 5 times for 2 min each with PBS containing 0.3% Triton X-100, followed by incubation with fluorescent-conjugated secondary antibodies diluted 1:1000 in blocking buffer for 2 h at room temperature. Secondary antibodies were goat anti-rabbit Alexa Fluor® 488 IgG (Abcam, Cat #ab150077). Samples were then rinsed 5 times for 2 min each with PBS containing 0.3% Triton X-100, followed by DAPI diluted in PBS at 1:5000 for 10 min. For experiments with icSARS-CoV-2-mNG in Calu3 cells, the cellular content for ROS was determined with the use of the fluorochrome Dihydroethidium (DHE, 5 μM) while nuclei were stained with 1 μg/ml Hoechst dye. Immunofluorescence (IF) images were obtained using an LSM700 or LSM880 Zeiss confocal microscope (Carl Zeiss GmbH, Jena, Germany). Immunofluorescence images were quantified using ImageJ software. DAPI was used to count total cell numbers in order to obtain a percentage of cells positive for spike protein. Alternatively, plates were imaged using the Operetta (PerkinElmer, Waltham, Mass.) system.

### Mitochondrial Reactive Oxygen Species Analysis

After 24 h (or 48 h, experiments involving 4F) post-infection, cells were collected and stained with 5 μM MitoSOX Red Mitochondrial reactive oxygen species (mito-ROS) indicator for 30 min at 37° C. Cells were washed with PBS, fixed with 4% p-formaldehyde for 30 min at 4° C. and transferred to polypropylene FACS tubes. Cells were then analyzed using an LSR Fortessa flow cytometer and FACSDiva software (Becton & Dickinson, San Diego, Calif., USA), and data were analyzed using FlowJo software.

### Flow Cytometry

SARS-CoV-2 infected cells were fixed with 4% paraformaldehyde for 20 min at 4° C. within the UCLA BSL3 high-containment facility and were transferred in polypropylene E-tubes to BSL2 containment facility for further processing. The cell pellets were resuspended in PBS and single cell suspensions were incubated with viability dye for 20 min in the dark at room temperature. Fixable Ghost Dye™ Red 780 (a cell viability dye) was used to exclude dead cells from analysis (Tonbo Biosciences). Appropriate antibodies were added to each tube and incubated in the dark for 20 min on ice. The following antibodies were used: PE/Cyanine7 mouse anti-human CD147 antibody (clone HIM6) was purchased from Biolegend (San Diego, Calif.). Mouse anti-human HO-1 antibody (clone HO-1-2) was purchased from Enzo Life Sciences and was conjugated with Mix-n-Stain CF647 Antibody Labeling Kit from Biotium Inc (Hayward, Calif.) according to the manufacturer's protocol. Rabbit anti-human MX1 polyclonal antibody was purchased from Proteintech (13750-1-AP). Rabbit anti-SARS-CoV-2 (2019-nCoV) Spike S1 Antibody (Clone R007, Cat #40150-R007) was purchased from Sino Biologicals and was conjugated with Mix-n-Stain CF488 Antibody Labeling Kit from Biotium Inc (Hayward, Calif.) according to the manufacturer's protocol. Mouse anti-SARS-CoV/SARS-CoV-2 spike S antibody (clone 1A9) was purchased from GeneTex. Alexa Fluor® 647 Cleaved Caspase-3 (Asp175) (Clone D3E9) was purchased from Cell Signaling Technology (Cat #9602S). Secondary antibodies were goat anti-rabbit Alexa Fluor® 488 IgG (Abcam, Cat #ab150077), goat anti-mouse Alexa Fluor® 546 (Thermo Fisher Scientific Cat #A11003), goat anti-rabbit DyLight® 650 (Abcam, Cat #ab96902).

After 20 min, the cells were washed twice with PBS. After a short spin, the cells were suspended in 200 μL of ice-cold PBS buffer and transferred to fresh tubes for FACS analysis. Samples were acquired using an LSR Fortessa flow cytometer and FACSDiva software (BD Biosciences). Instrument settings (cytosettings) for each protocol were tailored with unique voltage and compensation matrices. Verify tubes were used to track instrument settings over time. Data were analyzed using FlowJo software. At least 10,000 cells were acquired for each analysis, and each representative flow plot was repeated more than 3 times. Only live and singlet cells were chosen for analysis and gating (i.e., dead cells and aggregates were excluded). Single stain and also fluorescence minus one (FMO) controls were used in the presence of a given concentration of the antibody staining cocktail. Two readouts of protein expression were used: i) % of cells that were positive for the protein; ii) the median cellular amount of each protein per cell [median fluorescence intensity (MFI) minus the MFI of negative staining control. The difference in fluorescence intensity compared to the negative control (DMFI or % positive cells for each protein of parent cell population) was reported for each sample. Flow cytometry data among donors were obtained in parallel to avoid batch effects in stock solutions of dyes.

### Biomarkers of Inflammation

Protein levels of secreted IL-6 were determined in cell culture supernatants using ELISA kits according to the manufacturer (R&D). Protein levels of cytokines (IL-1 beta, IL-8, TNF-alpha, IL-10) in cell culture supernatants were determined using the human magnetic Luminex performance assay kits (LXSAHM) according to the manufacturer instructions (R&D).

### Statistics

Unless noted, error bars in all Figures represent mean and standard error of means (SEM). In the Figures, p-values are presented for comparisons between treatment groups and controls and are denoted by asterisks. To pool cells from different experiments, each measurement was first normalized to the vehicle controls of each experiment. Each experiment contains at least 3 biological replicates (number of wells) and each analysis contains at least 3 independent experiments. For flow analysis of cells, at least 10,000 events were acquired for the population of interest. For analysis of data that contains more than 2 groups, the Kruskal-Wallis test was performed to compare samples; if these comparisons had ap value less than 0.05 then Mann-Whitney U tests were used to compare statistical difference between 2 groups. p values less than 0.05 by Kruskal-Wallis or Mann-Whitney were considered significant. In the setting of exploratory approach, multiple comparisons were not adjusted because commonly used multiple testing adjustment methods assume independence of tests, which in protein expression studies and in the explored pathways herein translates to a questionable assumption that all explored measures operate independently. Instead, consistency between 2 independent experiments, direction, and magnitude of the correlation coefficient in conjunction with the nominal p values were considered in order to help distinguish true and false-positive findings. Consultation on statistical analysis was performed with the UCLA Biostatistics Department. All analyses were performed with Graphpad, version 8.0.

### Drug Treatments and Cytotoxicity Assays

Cultured cells were incubated separately with Mito-MES or decyl triphenyl phosphonium cation (TPP) (0, 10, 100, 250, 500 or 1000 nM)—a non-antioxidant mitochondria-targeted control compound which allowed for the non-specific effects of the TPP moiety of Mito-MES to be examined. The concentration of DMSO vehicle control was maintained constant at 0.01% v/v for all treatments. Drug effects were measured relative to vehicle controls in vitro. Unless stated, Calu3, Vero, HEK293-ACE2, and ALI cultures were pretreated for 16-24 h with the indicated treatments (Mito-MES Brusatol, Mito-TEMPO) or vehicle control. Then the cells were washed, infected with SARS-CoV-2 for 2 h, removed the virus, and added back the treatments (Mito-MES, Mito-TEMPO, remdesivir). Remdesivir (5 μM), a well characterized, direct acting antiviral agent, was used as an antiviral control.

4F Drug Treatments: Vero-E6 cells were plated at 20,000 cells/well and Calu3 were plated at 50,000 cells/well in a 96-well plate 48 h before infection. Medium containing a dose range of 4F vs vehicle (deionized water) controls were added 24 h before infection. At the end of this time, the cells were washed 3 times before viral infection. Cells were then adsorbed with MOI 0.1 PFU/cell of SARS-CoV-2 at 37° C. in serum free medium for one h. Plates were manually rocked every 10 min to redistribute the inoculum. After 60 min, virus inoculum was removed, cells were washed with Phosphate buffered saline (PBS) once to remove unbound virus, medium containing 4F or remdesivir or vehicle control (dH2O) was added back onto the cells, and cells were incubated for 24-48 h at 37° C. For the 0% inhibition control, cells were infected in the presence of vehicle only. Drug effects were measured relative to vehicle controls in vitro. To simulate the use of 4F as antiviral agent for preexposure prophylaxis, cells were treated with 4F triplicate per dilution for 24 h before infection. At 48 hpi cells and cell culture supernatants were harvested for downstream applications.

To measure cell viability to determine if there was any treatment-induced cytotoxicity, cells were plated and treated with drugs as described above. Cells were exposed to the same dilution series created for the in vitro efficacy studies. As above, vehicle-treated cells served as the 0% cytotoxicity control. After 24 h, cell viability was measured on a Synergy 2 Biotek microplate reader (Biotek Inc) via the XTT Cell Proliferation Assay Kit (ATCC® 30-1011K™) according to the manufacturer's protocol. Similar data were obtained in three independent experiments.

### Nasopharyngeal Swabs

One important consideration with COVID-19 testing is that it is often unclear how exactly the NP swab was obtained (by healthcare worker or not and whether the posterior or anterior nasopharynx were assessed). All NP swabs were done only by healthcare workers (including A1), minimizing the risk of inappropriate sampling technique. 5 NP swabs in A1 were obtained from the posterior nasopharynx while 3 swabs were midnasal and were performed in both nostrils. Two NP swabs in A2 were obtained from the posterior nasopharynx while one swab was midnasal. One NP swab obtained from each of C1 and C2 was from the posterior nasopharynx and one (per child) was mid-nasal. No oral swab was performed.

### SARS-CoV-2 PCR Diagnostic Assays

The SARS-CoV-2 PCR diagnostic assays utilized by the UCLA Clinical Microbiology Laboratory are the Simplexa (DiaSorin Molecular, Cypress, Calif.) and the TaqPath (Thermo Fisher Scientific, Waltham, Mass.) COVID-19 RT-PCR assays that have been authorized by FDA under an Emergency Use Authorization (EUA) for use by laboratories certified under the Clinical Laboratory Improvement Amendments (CLIA). The TaqPath COVID-19 RT-PCR assay is primarily used on ambulatory patients and low-risk health care workers because of its highest throughput. The Simplexa COVID-19 Direct Real-Time RT-PCR assay is primarily used on inpatients and high-risk health care workers because of its faster turnaround time. The TaqPath SARS-CoV-2 Assay was performed in four NP swabs from A1, two NP swabs from A2 and one NP swab from C1 and C2.

The reported sensitivity of the Simplexa PCR for NS is 70.8%-100% based on the amount of detectable viral genomic RNA. The reported Limit of Detection (LoD) for NS is 500 copies/ml. The reported sensitivity of the TaqPath PCR for NS is 100% based on the amount of detectable viral genomic RNA. The reported Limit of Detection (LoD) for NS is 10 copies per reaction. The Taqpath assay has higher sensitivity than the Simplexa PCR. In an evaluation of 107 weakly positive (any target CT >30) NP specimens the sensitivity of the TaqPath assay was 97.8% and the sensitivity of the Simplexa assay 75.3%.

The assays are standardized and were performed on NP swabs using methods in the art. The Simplexa COVID-19 Direct Real-Time RT-PCR assay was performed on the LIASON MDX instrument (DiaSorin Molecular). This assay targets the SARS-CoV-2 S and ORF1ab genes. Detection of one or both targets was deemed positive. The TaqPath COVID-19 RT-PCR assay targets the SARS-CoV-2 N, S, and ORF1ab genes. Nucleic acid extraction was performed with the MagMax Viral/Pathogen Nucleic Acid Isolation Kit using the automated KingFisher Flex Purification System (Thermo Fisher Scientific). RT-PCR was performed on the Applied Biosystems 7500 Fast Real-Time PCR instrument. Detection of two or all targets was deemed positive; detection of only one target was deemed inconclusive.

The Curative SARS-CoV-2 Assay (KorvaLabs, Inc., San Dimas, Calif.) COVID-19 RT-PCR assay has been authorized by FDA under an Emergency Use Authorization (EUA) for use by laboratories certified under the Clinical Laboratory Improvement Amendments (CLIA). The Curative SARS-CoV-2 Assay was performed in two NP swabs from A1 and one NP swab from each of A2, C1 and C2. The reported sensitivity of the Simplexa PCR for NS is 75%-100% based on the amount of detectable viral genomic RNA. The reported Limit of Detection (LoD) for NS is 100 copies/ml.

### Serological IgG Covid-19 Assay

The DiaSorin LIAISON SARS-CoV-2 S1/S2 IgG is the serological test utilized by the UCLA Clinical Microbiology Laboratory. It has reported diagnostic sensitivity 97.6% (95% CI 87.4%, 99.6%), specificity 99.3% (95% CI 98.6%, 99.6%) and NPV of 99.9% (95% CI 99.3%, 100%) (>15 days post-symptom onset).

### Exemplary Clinical Studies

### Preexposure and Postexposure Prophylaxis Studies

The following is an exemplary randomized placebo control trial for evaluating whether Mito-MES effectively provide pre- and/or post-exposure prophylaxis against the development of COVID-19.

In this design, Mito-MES and a placebo are administered to members of families at very high risk for developing COVID-19 as a pre-exposure prophylaxis against COVID-19. The participant families include those having at least two family members at high risk for exposure to COVID-19 (e.g., worker at groceries, teacher, children at school, health care worker) who are at risk for severe COVID-19 (e.g., one who has a comorbidity such as diabetes, obesity, hypertension, etc.). It is expected that social distancing practices will not be applied within the same household. Thus, several confounders will be minimized and efficacy of Mito-MES can be demonstrated with relatively small sample size.

The study goals will be to determine whether Mito-MES prevents, inhibits, and/or reduces infections by SARS-CoV-2 and Mito-MES prevents inhibits or reduces the development of severe COVID-19.

Mito-MES or placebo is administered blindly to family members within the same family. At least 200 participants per group will be enrolled (e.g., 2 participants per family where one is administered placebo and the other is administered Mito-MES=100 families per group). Participants of the pre-exposure prophylaxis group will be administered 20 mg/day of Mito-MES throughout the study. In the placebo group, if a family member becomes infected with SARS-CoV-2, one participant of that family will be administered Mito-MES (forming the post-exposure prophylaxis group) and the other will continue being administered placebo (forming the control group). Each participant will be monitored for 3 months post enrollment.

It is expected that Mito-MES will be more effective as a pre-exposure prophylactic than a post-exposure prophylactic against SARS-CoV-2.

It is also expected that the efficacy of Mito-MES as a post-exposure prophylactic against the development of severe COVID-19 will be higher when administered early, e.g., within 4 days of exposure to a subject, who at the time of the exposure had an active and confirmed SARS-CoV-2 infection.

### Mito-MES for Treating COVID-19

The following is an exemplary randomized placebo control trial for determining whether Mito-MES is effective in treating COVID-19.

Mito-MES vs placebo will be given within 4 days after a positive test for SARS-CoV-2 and participants will be monitored for 3 weeks for the development of severe COVID-19.

100 participants per group (Mito-MES 20 mg/day vs placebo) will be enrolled. Additional treatment arms may include different doses of Mito-MES (e.g., 10 mg/day vs 30 mg/day vs 40 mg/day vs 50 mg/day vs 60 mg/day vs 70 mg/day vs 80 mg/day).

It is expected that the efficacy of Mito-MES in treating acute and severe COVID-19 will be higher when administered early, e.g., within 4 days of exposure to a subject, who at the time of the exposure had an active and confirmed SARS-CoV-2 infection. Given the antiviral and anti-inflammatory action of Mito-MES it is expected that Mito-MES will attenuate (i.e., prevent, inhibit, or reduce) the development and progression of severe COVID-19, which typically occurs about 7-14 days post exposure.

### Mito-MES Treating Chronic COVID-19 Syndrome

The following is an exemplary randomized placebo control trial for determining the efficacy of Mito-MES in treating chronic COVID-19 syndrome.

The primary goal be to determine whether Mito-MES is effective in reducing the symptoms of chronic COVID-19 syndrome (diagnosed based on persistence of chronic symptoms 3 weeks post COVID-19 infection).

Participants having moderate-to-severe COVID-19 and at least 2 comorbidities will be administered 20 mg/day Mito-MES or placebo and monitored for 3 months. At least 100 participants per group will be enrolled. Additional treatment arms may include different doses of Mito-MES (e.g., 10 mg/day vs 30 mg/day vs 40 mg/day vs 50 mg/day vs 60 mg/day vs 70 mg/day vs 80 mg/day).

It is expected that Mito-MES will effectively reduce the symptoms of chronic COVID-19 syndrome.

### REFERENCES

The following references are herein incorporated by reference in their entirety with the exception that, should the scope and meaning of a term conflict with a definition explicitly set forth herein, the definition explicitly set forth herein controls:
Hu, et al. (2021) Characteristics of SARS-CoV-2 and COVID-19. Nat Rev Microbiol 19:141-154.
Ansems, et al. (2021) Remdesivir for the treatment of COVID-19. Cochrane Database Syst Rev 8:CD014962.
Cain, et al. (2017) Immune regulation by glucocorticoids. Nat Rev Immunol 17:233-247.
Lee, et al. (2020) Antibody-dependent enhancement and SARS-CoV-2 vaccines and therapies. Nat Microbiol 5:1185-1191.
Zhou, et al. (2021) beta-d-N4-hydroxycytidine Inhibits SARS-CoV-2 Through Lethal Mutagenesis But Is Also Mutagenic To Mammalian Cells. J Infect Dis 224:415-419.
Hu, et al. (2019) Respiratory syncytial virus co-opts host mitochondrial function to favour infectious virus production. Elife 8.
Shin, et al. (2019) Immune Responses to Middle East Respiratory Syndrome Coronavirus During the Acute and Convalescent Phases of Human Infection. Clin Infect Dis 68:984-992.
Braakhuis, et al. (2018) The Effect of MitoQ on Aging-Related Biomarkers: A Systematic Review and Meta-Analysis. Oxid Med Cell Longev 2018:8575263.
Smith, et al. (2010) Animal and human studies with the mitochondria-targeted antioxidant MitoQ. Ann N Y Acad Sci 1201:96-103.
Rossman, et al. (2018) Chronic Supplementation With a Mitochondrial Antioxidant (MitoQ) Improves Vascular Function in Healthy Older Adults. Hypertension 71:1056-1063.
Tones, et al. (2015) D. X. Liu:Protein-Protein Interactions of Viroporins in Coronaviruses and Paramyxoviruses: New Targets for Antivirals? Viruses 7:2858-2883.
Mullen, et al. (2021) SARS-CoV-2 infection rewires host cell metabolism and is potentially susceptible to mTORC1 inhibition. Nat Commun 12:1876.
Codo, et al. (2020) Elevated Glucose Levels Favor SARS-CoV-2 Infection and Monocyte Response through a HIF-1alpha/Glycolysis-Dependent Axis. Cell Metab 32:437-446 e435.
James, et al. (2007) Interaction of the mitochondria-targeted antioxidant MitoQ with phospholipid bilayers and ubiquinone oxidoreductases. J Biol Chem 282:14708-14718.
Liu, et al. (2010) Tom70 mediates activation of interferon regulatory factor 3 on mitochondria. Cell Res 20:994-1011.
Sena, et al. (2012) Physiological roles of mitochondrial reactive oxygen species. Mol Cell 48:158-167.
Bizzotto, et al. (2020) SARS-CoV-2 Infection Boosts MX1 Antiviral Effector in COVID-19 Patients. iScience 23:101585.
Cao, et al. (2020) The anti-viral dynamin family member MxB participates in mitochondrial integrity. Nat Commun 11:1048.
Moolamalla, et al. (2021) Host metabolic reprogramming in response to SARS-CoV-2 infection: A systems biology approach. Microb Pathog 158:105114.
Zielonka, et al. (2017) and Therapeutic and Diagnostic Applications. Chem Rev 117:10043-10120.
Dikalova, et al. (2010) Therapeutic targeting of mitochondrial superoxide in hypertension. Circ Res 107:106-116.
Rodriguez-Cuenca, et al. (2010) Consequences of long-term oral administration of the mitochondria-targeted antioxidant MitoQ to wild-type mice. Free Radic Biol Med 48:161-172.
Fink, et al. (2012) Bioenergetic effects of mitochondrial-targeted coenzyme Q analogs in endothelial cells. J Pharmacol Exp Ther 342:709-719.
Hu, et al. (2018) The mitochondrially targeted antioxidant MitoQ protects the intestinal barrier by ameliorating mitochondrial DNA damage via the Nrf2/ARE signaling pathway. Cell Death Dis 9:403.
Saidu, et al. (2017) Dimethyl Fumarate Controls the NRF2/DJ-1 Axis in Cancer Cells: Therapeutic Applications. Mol Cancer Ther 16:529-539.
Saha, et al. (2020) An Overview of Nrf2 Signaling Pathway and Its Role in Inflammation. Molecules 25.
Espinoza, et al. (2017) Modulation of Antiviral Immunity by Heme Oxygenase-1. Am J Pathol 187:487-493.
Olagnier, et al. (2020) SARS-CoV2-mediated suppression of NRF2-signaling reveals potent antiviral and anti-inflammatory activity of 4-octyl-itaconate and dimethyl fumarate. Nat Commun 11:4938.
White, et al. (2021) Plitidepsin has potent preclinical efficacy against SARS-CoV-2 by targeting the host protein eEF1A. Science 371:926-931.
Winkler, et al. (2020) SARS-CoV-2 infection of human ACE2-transgenic mice causes severe lung inflammation and impaired function. Nat Immunol 21:1327-1335.
Dashdorj, et al. (2013) Mitochondria-targeted antioxidant MitoQ ameliorates experimental mouse colitis by suppressing NLRP3 inflammasome-mediated inflammatory cytokines. BMC Med 11:178.
Lowes, et al. (2013) and reduce biochemical markers of organ dysfunction in a rat model of acute sepsis. Br J Anaesth 110:472-480.
Nalbandian, et al. (2021) Post-acute COVID-19 syndrome. Nat Med 27(4):601-615.

The structural formula of the triphenylphosphonium moiety (“TPP moiety”) is as follows:

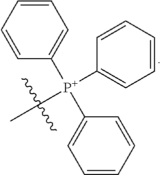 [SMILES: *[P+](c1ccccc1)(c1ccccc1)c1ccccc1]

As used herein, a Triphenylphosphonium Compound (“TPP Compound”) refers to a compound that has the TPP moiety as part of its structural formula. Exemplary TPP Compounds include TPP Hydrocarbons and TPP Conjugates. As used herein, “TPP Hydrocarbons” have a saturated or unsaturated, substituted or unsubstituted, branched or unbranched hydrocarbon (HC) group attached to the phosphonium ion. As used herein, “TPP Conjugates” are compounds having a bioactive moiety, i.e., a chemical moiety that exhibits bioactivity by itself, conjugated to the TPP moiety via a linker that is a saturated or unsaturated, substituted or unsubstituted, branched or unbranched hydrocarbon group attached to the phosphonium ion. In some embodiments, the hydrocarbon group is a C₁-C15 alkyl, alkenyl, or alkynyl group. In some embodiments, the hydrocarbon group is a C₁-C₁₀ alkyl, alkenyl, or alkynyl group. In some embodiments, the hydrocarbon group is a C₁-C₁₀ unbranched alkyl group. Exemplary TPP Conjugates include Mito-MES and those set forth in FIG. 20-a: Mito-Quinone, Mito-Vitamin E, Mito-CarboxyProxyl, Mito-Tempol, Mito-Honokiol, Mito-Apocynin, Mito-Resveratrol, Mito-Vitamin C, Mito-Metformin, Mito-SNO, AP39, Mito-Ebselen, Mito-Doxorubicin, Mito-Geldamycin, Mito-15d-PGJ2, Mito-Dichloroacetate, Mito-Chlorambucil, Mito-Curcumin, Mito-PhotoDNP, Mito-Octyne, Mito-DIPPMPO, Mito-HE, o-MitoPhB(OH)₂, Mito-PY1, Mito-Porphyrin, Mito-^{99m}Tc-MAG3, Mito-Gd-DOTA, and [¹⁸F]-FBnTP.

Bioactive moieties may be antioxidants (e.g., tocopherol, ubiquinone, thymoquinone, plastoquinone, etc.) and/or Nrf2 agonists (curcumin, resveratrol, etc.). Exemplary bioactive moieties include ubiquinone and derivatives thereof (i.e., compounds having 2,3-dimethoxy-5-methylcyclohexa-2,5-diene-1,4-dione as part of its chemical structure), Vitamin E, CarboxyProxyl (1-hydroxy-2,2,5,5-tetramethylpyrrolidine-3-carboxylic acid), Tempol, Honokiol, Apocynin, Resveratrol, Vitamin C, Metformin, S-nitrosothiol and compounds containing S-nitrosothiol as part of its chemical structure, dithiolethione, Ebselen, Doxorubicin, Geldamycin, 15d-PGJ2, Dichloroacetate, Chlorambucil, Curcumin, PhotoDNP (6-[(4-azido-2-nitrophenyl)amino]-N-{6-[(2,4-dinitrophenyl)amino]hexyl}hexanamide), Octyne, DIPPMPO, dihydroethidium, phenylboronic acid, dipolar 1,3,6,8-tetrasubstituted pyrene-based compounds (e.g., PY1, PY2, MPY1, MPY2, etc.), porphyrin, 99mTc-MAG3, and Gd-DOTA. A bioactive moiety may be provided as a distinct compound that is not conjugated to the TPP moiety.

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

As used herein, the term “diagnosing” refers to the physical and active step of informing, i.e., communicating verbally or by writing (on, e.g., paper or electronic media), another party, e.g., a patient, of the diagnosis. Similarly, “providing a prognosis” refers to the physical and active step of informing, i.e., communicating verbally or by writing (on, e.g., paper or electronic media), another party, e.g., a patient, of the prognosis.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, “co-administered” refers to the administration of at least two different agents, e.g., a mitochondrial targeted antioxidant and an ApoA-I mimetic peptide, to a subject. In some embodiments, the co-administration is concurrent. In embodiments involving concurrent co-administration, the agents may be administered as a single composition, e.g., an admixture, or as two separate compositions. In some embodiments, the first agent is administered before and/or after the administration of the second agent. Where the co-administration is sequential, the administration of the first and second agents may be separated by a period of time, e.g., minutes, hours, or days.

As used herein, the phrase “consisting essentially of” in the context of a given ingredient in a composition, means that the composition may include additional ingredients so long as the additional ingredients do not adversely impact the activity, e.g., biological or pharmaceutical function, of the given ingredient. For example, a composition that “consisting essentially of” a mitochondrial targeted antioxidant means that the may comprise additional ingredients so long as the additional ingredients do not adversely affect the activity of the mitochondrial targeted antioxidant. As another example, a composition that “consists essentially of (a) one or more mitochondrial targeted antioxidants and (b) one or more ApoA-I mimetic peptides” means that the composition may comprise additional ingredients so long as the additional ingredients do not adversely affect the activity one or more mitochondrial targeted antioxidants and the one or more ApoA-I mimetic peptides. Such additional ingredients may include pharmaceutically acceptable carriers, buffers, binders, preservatives, wetting agents, emulsifying agents, dispersing agents, etc.

The phrase “comprising, consisting essentially of, or consisting of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of preventing, inhibiting, or reducing (a) an infection by, (b) an inflammatory response caused by, (c) apoptosis caused by a coronavirus in a cell or a subject, or (d) treating a subject for a coronavirus disease caused by infection by a coronavirus, which comprises administering one or more triphenylphosphonium compounds (TPP Compounds) to the cell or the subject.

2. The method according to claim 1, which further comprises administering to the cell or the subject one or more bioactive moieties, one or more ApoA-I mimetic peptides, and/or one or more Nrf2 agonists.

3. (canceled)

4. The method according to claim 1, wherein a therapeutically effective amount of the one or more TPP Compounds of about 0.05 mg/kg to about 15 mg/kg, preferably about 0.2 mg/kg to about 1.5 mg/kg, or more preferably about 0.3 mg/kg to about 0.7 mg/kg, weight of the subject is administered to the subject.

5. The method according to claim 1, wherein a therapeutically effective amount of the one or more TPP Compounds of about 1-1000 mg, 5-100 mg, 10-80 mg, or 20-40 mg, preferably about 20 mg, is administered to the subject.

6. The method according to claim 1, wherein the one or more TPP Compounds is administered daily.

7. The method according to claim 1, wherein the one or more TPP Compounds is administered orally, subcutaneously, or intravenously, preferably orally.

8. The method according to claim 1, wherein the coronavirus is SARS-CoV-2.

9. The method according to claim 1, wherein the one or more TPP Compounds is mitoquinol mesylate or mitoquinone mesylate.

10. The method according to claim 1, wherein the administration of the one or more TPP Compounds is before, during, and/or after the subject was exposed or likely exposed to the coronavirus.

11. The method according to claim 10, wherein the administration of the one or more TPP Compounds occurs for at least 1-10 days after the exposure or likely exposure to the coronavirus.

12. A method of preventing, inhibiting, or reducing infection by a coronavirus in a subject, which comprises providing a plasma concentration of about 2 ng/ml or higher of one or more triphenylphosphonium compounds (TPP Compounds) in the subject during and/or after the subject is exposed to the coronavirus.

13. (canceled)

14. A composition comprising, consisting essentially of, or consisting of (a) one or more triphenylphosphonium compounds (TPP Compounds), and (b) one or more bioactive moieties, one or more ApoA-I mimetic peptides, and/or one or more Nrf2 agonists.

15. The composition according to claim 14, wherein the one or more TPP Compounds is selected from mitoquinone and salts thereof, mitoquinol and salts thereof, SkQ1, Elamipretide, Mito-TEMPO; the one or more ApoA-I mimetic peptides is selected from 4F, D-4F, reverse D-4F, and 6F; and the one or more Nrf2 agonists is selected from Antcin C, Baicalein, Butein and phloretin, Carthamus red, Curcumin, Diallyl disulfide, Ellagic acid, Gastrodin, Ginsenoside Rg1, Ginsenoside Rg3, Glycyrrhetinic acid, Hesperidin, Isoorientin, Linalool, Lucidone, Lutein, Lycopene, Mangiferin, Naringenin, Oleanolic acid, Oroxylin A, Oxyresveratrol, Paeoniflorin, Puerarin, Quercetin, Resveratrol, S-Allylcysteine, Salvianolic acid B, Sauchinone, Schisandrin B, Sulforaphane, Tungtungmadic acid, Withaferin A, Alpha-lipoic acid, and Dimethyl fumarate (DMF).

16. The composition according to claim 15, wherein the one or more bioactive moieties is an antioxidant, an Nrf2 agonist, or a compound selected from the group consisting of ubiquinone and derivatives thereof (i.e., compounds having 2,3-dimethoxy-5-methylcyclohexa-2,5-diene-1,4-dione as part of its chemical structure), Vitamin E, CarboxyProxyl (1-hydroxy-2,2,5,5-tetramethylpyrrolidine-3-carboxylic acid), Tempol, Honokiol, Apocynin, Resveratrol, Vitamin C, Metformin, S-nitrosothiol and compounds containing S-nitrosothiol as part of its chemical structure, dithiolethione, Ebselen, Doxorubicin, Geldamycin, 15d-PGJ2, Dichloroacetate, Chlorambucil, Curcumin, PhotoDNP (6-[(4-azido-2-nitrophenyl)amino]-N-{6-[(2,4-dinitrophenyl)amino]hexyl}hexanamide), Octyne, DIPPMPO, dihydroethidium, phenylboronic acid, dipolar 1,3,6,8-tetrasubstituted pyrene-based compounds (e.g., PY1, PY2, MPY1, MPY2, etc.), porphyrin, 99mTc-MAG3, and Gd-DOTA.

17. The composition according to claim 16, wherein the one or more TPP Compounds is a TPP Hydrocarbon or a TPP Conjugate.

18. The composition according to claim 17, wherein the one or more TPP Compounds is not Mito-MES, MitoQ™, SkQ1 (Mitotech, S.A.), Elamipretide (Stealth BioTherapeutics), Mito-TEMPO (CAS 1569257-94-8) or a compound disclosed in U.S. Pat. Nos. 8,518,915; 9,192,676; 9,328,130; 9,388,156; US20070161609; US20070225255; US20080161267; US20100168198; US20160200749; US20180305328; US20190248816; US20190330249; US20190374558; WO2005019232; WO2006005759; WO2007046729; WO2008145116; WO2015063553; WO2017106803; and WO2018162581.

19. The composition according to claim 17, wherein the one or more TPP Compounds is (1-decyl)triphenylphosphonium.

20. The composition according to claim 17, wherein the one or more TPP Compounds is (1-decyl)triphenylphosphonium, which is provided in the form of a mixture with coenzyme Q10.
